(19) United States
(12) Patent Application Publication
(10) Pub. No.: US 2022/0401640 A1
(43) Pub. Date: Dec. 22, 2022

(54) INTRAVENOUS INFUSION PUMPS WITH SYSTEM AND PHARMACODYNAMIC MODEL ADJUSTMENT FOR DISPLAY AND OPERATION

(71) Applicant: ICU Medical, Inc., San Clemente, CA (US)

(72) Inventor: James D. Jacobson, Lindenhurst, IL (US)

(21) Appl. No.: 17/806,847

(22) Filed: Jun. 14, 2022

Related U.S. Application Data
(60) Provisional application No. 63/211,905, filed on Jun. 17, 2021.

(51) Int. Cl. A61M 5/142

(52) U.S. Cl. CPC A61M 5/142; A61M 2005/14208

(57) ABSTRACT

An infusion pump can determine and display what the drug load is inside of a patient by taking into account various factors such as drug half-life, pump pauses, and delays while a drug moves from a pump to a patient. A pump can also calculate and provide times when: medication will reach the patient; the drug concentration will reach a specified level; and a physiological response is expected. The pump can compensate for pauses in the delivery—for example, by infusing larger boluses of the drug into the patient within safe boundaries for concentration and timing. The pump can also predict what the drug load or concentration will be in the patient over time after the infusion stops by providing a graph, and in some cases act on such predictions by changing a flow rate or other parameters.

### INCORPORATION BY REFERENCE TO PRIORITY APPLICATION

This application is based upon and claims the benefit of priority from U.S. Provisional Patent Application No. 63/211,905, filed on Jun. 17, 2021. Moreover, any and all applications for which a foreign or domestic priority claim is identified in the Application Data Sheet as filed with the present application are hereby incorporated by reference under 37 CFR § 1.57. The entire contents of each of the above-listed items is hereby incorporated into this document by reference and made a part of this specification for all purposes, for all that each contains.

### BACKGROUND

### Field

This disclosure relates to intravenous infusion pumps, including electronically controlled intravenous infusion pumps.

### Related Art

Pumps for infusing medications suffer from various drawbacks. For example, commercial intravenous infusion pumps allow selection and display of an infusion rate that may not reflect or be helpful in discerning actual drug levels in a patient.

### SUMMARY

A selected infusion rate in an electronic intravenous infusion pump sometimes does not match closely with the actual infusion rate or allow prediction of when a drug will achieve equilibrium or produce a clinical effect in a patient. This disclosure provides solutions to this problem, including modeling and improved systems for smart pumps and improved displays and controls to assist clinicians and improve patient care.

### BRIEF DESCRIPTION OF THE DRAWINGS

The following drawings and the associated descriptions are provided to illustrate embodiments of the present disclosure and do not limit the scope of the claims.

FIGS. 1A-E show front perspective, front elevational, rear elevational, top plan, and side elevational views, respectively, of an example of an infusion pump.

FIG. 2A shows an example of a cassette that can be used with the pump of FIG. 1.

FIGS. 2B, 2C, and 2D illustrate three cross-section views of a cassette similar to FIG. 2A.

FIG. 3A illustrates infusion mechanism hardware for interacting with the cassette of FIGS. 2A-2D.

FIG. 3B illustrates a fluid path through a cassette such as those shown in FIGS. 2A-2D, as controlled by the hardware of FIG. 3A.

FIG. 3C illustrates schematically how hardware (e.g., FIG. 3A) interacts with a cassette (e.g., FIGS. 2A-2D) to affect flow along a fluid path.

FIG. 3D shows a schematic diagram of functional components for an example of a medical pump system.

FIG. 4A shows a schematic for a valve actuation motor.

FIG. 4B schematically depicts components of an electric motor for an infusion pump.

FIG. 4C shows a schematic for a plunger drive motor.

FIG. 5A is a plunger motor position diagram.

FIG. 5B is a schematic block diagram of an example infusion pump with a plunger.

FIG. 6A is a schematic block diagram of a pump, showing a driven plunger in a home position relative to an elastomeric membrane covered pumping chamber;

FIG. 6B is a schematic block diagram of a pump, showing the driven plunger in a retracted position relative to the elastomeric membrane covered pumping chamber;

FIG. 6C is a schematic block diagram of a pump, showing the driven plunger in an advanced position relative to the elastomeric membrane covered pumping chamber;

FIG. 7 is a schematic diagram of flow line features in a multi-line pump with feedback and control.

FIG. 8 shows section view snapshots of a pumping chamber, piston and elastic membrane interaction.

FIG. 9 shows a pump diagram with a sensor placed for pumping chamber feedback.

FIG. 10 plots force data versus angular plunger position from a pump cycle.

FIG. 11 shows a method for pump control.

FIG. 12 shows almost three hours of low flow pump continuity data averaging greater than 0.1 ml/hr.

FIG. 13 is a schematic diagram of a medication management system including a medication management unit and a medical device.

FIG. 14 is a schematic diagram of a medication management unit with a network interface.

FIG. 15 is a schematic diagram of a medical device, electronic network, MMU, and hospital environment.

FIG. 16 shows a plan view of a multi-line medical device and graphic user interface.

FIG. 17 shows graphic user interface and control features for a medical device.

FIG. 18 shows a graphical user interface for configuring aspects of an infusion or pump system and interacting with drug library information.

FIG. 19 shows a detail from the interface of FIG. 18.

FIG. 20 plots infusion volume over time and corresponding infusion rates.

FIG. 21 shows a schematic diagram of a pumping system that has sensors.

FIG. 22 shows a control and feedback system that can account for how a medication is consumed through biological processes and for mechanical infusion pauses.

FIG. 23 shows how a system can operate to improve infusion and related display.

FIG. 24 shows a simplified view of pump operation with steady infusion rates.

FIG. 25 shows how infusion pumps actually operate at low rates.

FIG. 26 shows low flow continuity curves for three sample infusion pumps.

FIG. 27 shows a plot modeling an impulse dose delivery.

FIG. 28 shows a plot modeling multiple doses.

FIG. 29A shows a relatively smooth plot modeling continuous delivery of medication to reach an equilibrium.

FIG. 29B shows a plot modeling a series of doses reaching an equilibrium.

FIG. 30A shows a plot modeling a series of doses reaching an equilibrium, with markers highlighting two times of interest

FIG. 30B shows a potential infusion rate change sequence driven by incorrect clinician knowledge of pump system operation and medication half life

FIG. 31 shows a plot of pulsed doses and a one-minute pause.

FIG. 32 shows a plot of pulsed doses, a pause, and a limited correction bolus.

FIG. 33 shows a plot of pulsed doses, a pause, and a bolus of all missed doses.

FIG. 34 shows a plot of pulsed doses, a long pause, and a bolus of all missed doses.

FIG. 35 shows how large volume pumps generally exhibit larger flow resolution than syringe pumps.

FIG. 36 shows a plot of syringe pump low flow continuity compared to other pumps.

FIG. 37 shows how syringe pumps often operate at low rates, demonstrating divergence from intended pulsed delivery.

FIG. 38 shows syringe pump startup curves for two syringe pump examples.

FIG. 39 shows a method of tracking pump operation details and other ex-vivo inputs for determining expected in-vivo infusate.

FIG. 40 depicts a self-aware infusion pump and display system.

### DETAILED DESCRIPTION

Patients all over the world who are in need of medical care would benefit from intravenous infusion therapy, especially during surgery or when hospitalized. This generally involves inserting a needle into a patient's blood vessel, usually in the hand or arm, and then coupling the needle to a catheter in communication with one or more different types of therapeutic fluids. Once connected, the fluid travels from the fluid source(s), through the catheter, and into the patient. The fluid can provide certain desired benefits to the patient, such as maintaining hydration or nourishment, diminishing infection, reducing pain, lowing the risk of blood clots, maintaining blood pressure, providing chemotherapy, and/or delivering any other suitable drug or other therapeutic liquid to the patient. Electronic infusion pumps in communication with the fluid sources and the patient can help to increase the accuracy and consistency of fluid delivery to patients, but current electronic infusion pumps have disadvantages

Although certain preferred embodiments and examples are disclosed below, inventive subject matter extends beyond the specifically disclosed embodiments to other alternative embodiments and/or uses and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

This specification provides textual descriptions and illustrations of many devices, components, assemblies, and subassemblies. Any structure, material, function, method, or step that is described and/or illustrated in one example can be used by itself or with or instead of any structure, material, function, method, or step that is described and/or illustrated in another example or used in this field. The text and drawings merely provide examples and should not be interpreted as limiting or exclusive. No feature disclosed in this application is considered critical or indispensable. The relative sizes and proportions of the components illustrated in the drawings form part of the supporting disclosure of this specification, but should not be considered to limit any claim unless recited in such claim.

The systems and methods discussed herein can be used anywhere, including, for example, in laboratories, hospitals, healthcare facilities, intensive care units (ICUs), or residences. Moreover, the systems and methods discussed herein can be used for invasive techniques, as well as non-invasive techniques or techniques that do not involve a body or a patient such as, for example, in vitro techniques.

In many commercial IV infusion pumps on the market today, the pump is generally programmable to infuse medication into the patient at a prescribed rate (such as mL/hr). However, this infusion rate does not tell the nurse what the true, instantaneous drug load or drug concentration (e.g., drug level) is inside of the patient's blood stream. The infusion rate is related to the drug load or concentration, but there are many intervening factors that can vary the patient's drug load or concentration over time. For example, if there is a primed tubing set and catheter filled with other fluid (typically saline before infusion begins) between the pump and the patient, this will typically delay the infusion of the drug or any change in the drug concentration into the patient. Another factor is drug half-life. Each drug has a characteristic half-life over which it breaks down and is eliminated from the blood stream by the patient's body, which in combination with the rate at which the drug is infused into the patient determines the overall concentration of the drug in the blood stream. Another factor is pauses in the infusion of medication from the pump to the patient. These pauses may be an inherent pump feature, used to achieve low flow rates. These pauses may also be used to eliminate air or a kink in the line or to replace an IV bag or syringe. Thus, drug input and drug half-life do not always easily achieve or maintain an equilibrium or steady state level, although that is often the goal for continuously infused medications.

Described is a system (e.g., an infusion pump) that can determine and display (e.g., convey to the nurse or doctor) what the drug load or concentration is inside of the patient at any given moment by taking into account various factors such as those described above. The factors can include: (a) the characteristic half-life for each particular drug (e.g., from a dynamic or previously-stored look-up table in an electronic drug library) in combination with the infusion rate; (b) the duration of a pause for clearing air, removing a kink, or replacing the bag or syringe; and/or (c) delays caused by how long it takes the drug or a change in the concentration of a drug to move from the pump through the catheter into the patient. In a related way, the pump can also calculate and provide (e.g., display or report to the patient, doctor or nurse) various significant times: (a) the time when the medication will first reach the patient; (b) the time when the drug load or concentration will reach a specified target level (and thereafter remain at equilibrium during a constant flow rate, such that the infusion rate is approximately equal to the half-life break-down of the drug in the patient's blood); and (c) the time when the patient is expected to achieve a particular physiological response to the medication. The pump can also compensate for pauses in the delivery—for example, by infusing larger boluses of the drug into the patient within safe boundaries for concentration and timing. The pump can also predict what the drug load or concentration will be in the patient over time after the infusion stops by providing a graph, and in some cases act on such predictions by changing a flow rate or other parameters. Such systems can convey to a clinician an expected or calculated amount for the current in-patient drug level. This can be expressed as a percentage of a desired steady state or equilibrium level. More general information can also be conveyed to the clinician: for example, that the infused medication has not yet been infused into the patient, that the medication is being infused into the patient but is not yet expected to have achieved a steady state level, or that the medication amount is expected to have reached a steady state level.

### Examples of Pump Systems

In some embodiments, a pump system can include a reusable pump driver and a disposable fluid holder, such as a fluid cassette, syringe, section of tubing, etc. A disposable cassette, which is typically adapted to be used for a single patient and/or for a limited time, is usually a small plastic unit having at least one inlet and an outlet respectively connected through flexible tubing to the fluid supply container and to the patient receiving the fluid. In some embodiments, the cassette can include a pumping chamber. The flow of fluid through the chamber can be controlled by a plunger or pumping element activated in a controlled manner by the reusable pump driver. For example, the cassette chamber can have one wall formed by a flexible diaphragm against which the plunger is repeatedly pressed in a reciprocating manner, which causes the fluid to flow. The pump driver can include the plunger or pumping element for controlling the flow of fluid into and out of the pumping chamber in the cassette, and it may also include one or more controls and/or vents to help deliver the fluid to the patient at a pre-set rate, in a pre-determined manner, for a particular pre-selected time, and/or at a pre-selected total dosage.

In some embodiments, the fluid can enter a cassette through an inlet and can be forced through an outlet under pressure. The fluid is delivered to the outlet when the pump plunger forces the membrane into the pumping chamber to displace the fluid. During the intake stroke, the pump plunger draws back, the membrane covering the pumping chamber retracts or pulls back from its prior inwardly displaced position, and the fluid is then drawn through the open inlet and into the pumping chamber. In a pumping stroke, the pump plunger forces the membrane back into the pumping chamber to force the fluid contained therein through the outlet. Some pumps can thus use passive valves to support pumping. However, active valves can also be timed to open or close simultaneously with appropriate portions of a pumping stroke cycle. By repeating the pumping actions in an electronically controlled manner, the fluid flows into and out of the cassette. Typically at lower rates, during the pumping cycle, the pump plunger is stepped in a specified timing sequence to deliver successive pulses of fluid from the pump chamber. For example, a single draw motion can correspond to a long series of small expel motions. Thus, the fluid can flow from the cassette in a series of spaced-apart pulses rather than an uninterrupted flow. When the pulses occur in rapid succession, the flow approximates a continuous flow. At higher rates the pump can typically displace fluid in a smoothly continuous manner. The entire disclosure of U.S. Pat. No. 7,258,534 is incorporated by reference herein, for all purposes, for all that it contains, including but not limited to examples of pump drivers and disposable fluid holders. It is contemplated that any structure, material, function, method, or step that is described and/or illustrated in the '534 patent can be used with or instead of any structure, material, function, method, or step that is described and/or illustrated in the text or drawings of this specification.

One feature of most medical pumps is that they can deliver precise volumes at precise delivery rates. Conventional pumps, in general, rely on nominal or empirical data to estimate the delivery volumes and delivery rates, and do not provide mechanisms for adjusting an actual delivery due to variations from this nominal or empirical data. Other pumps utilize enhanced sensing during infusion to enhance operational accuracy.

### Example Pump Components

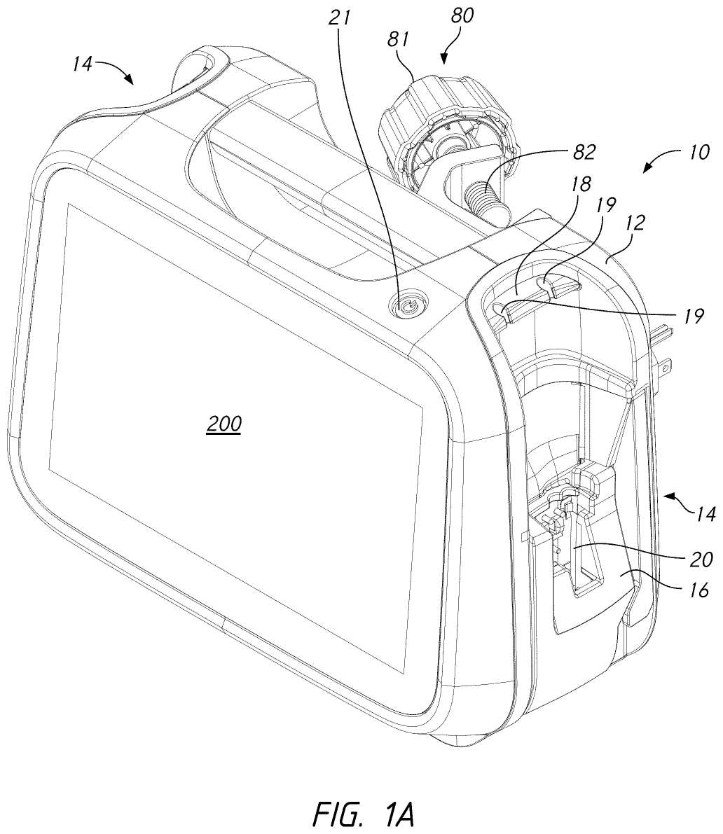

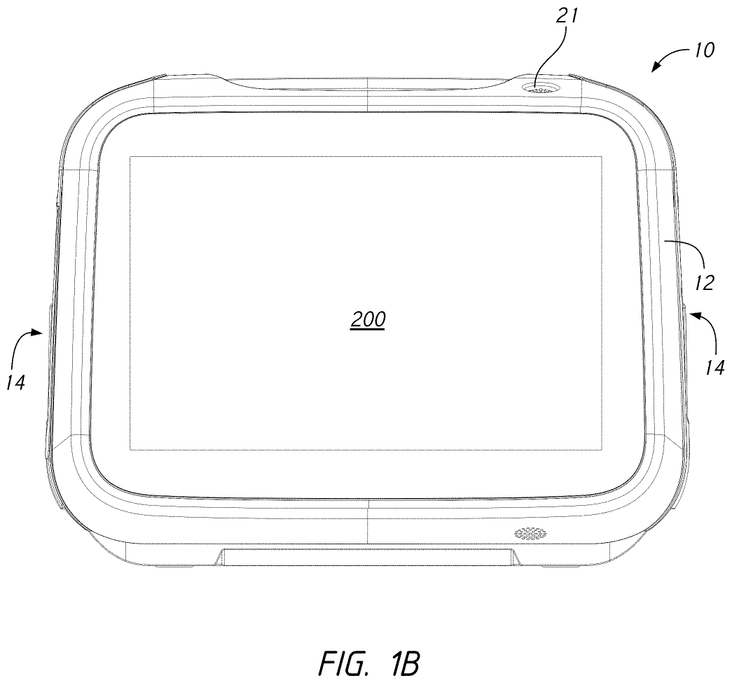

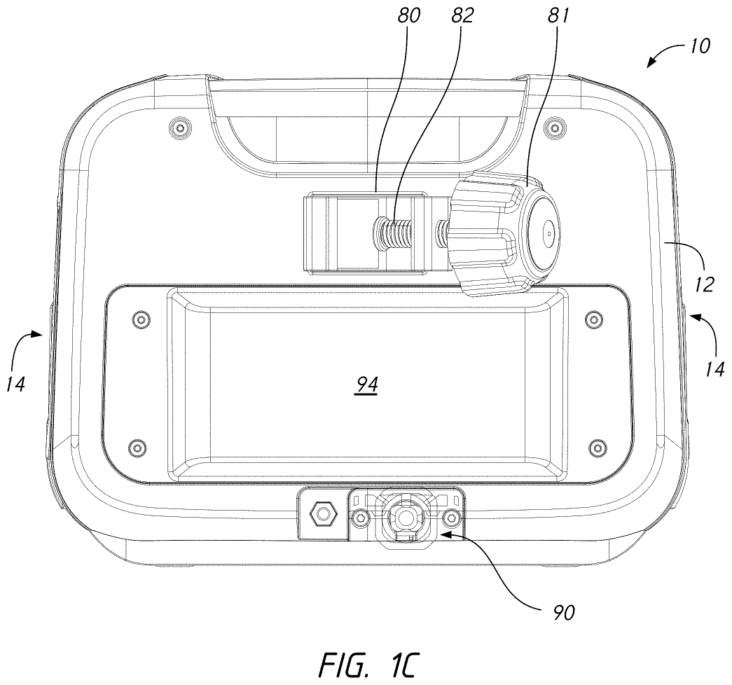

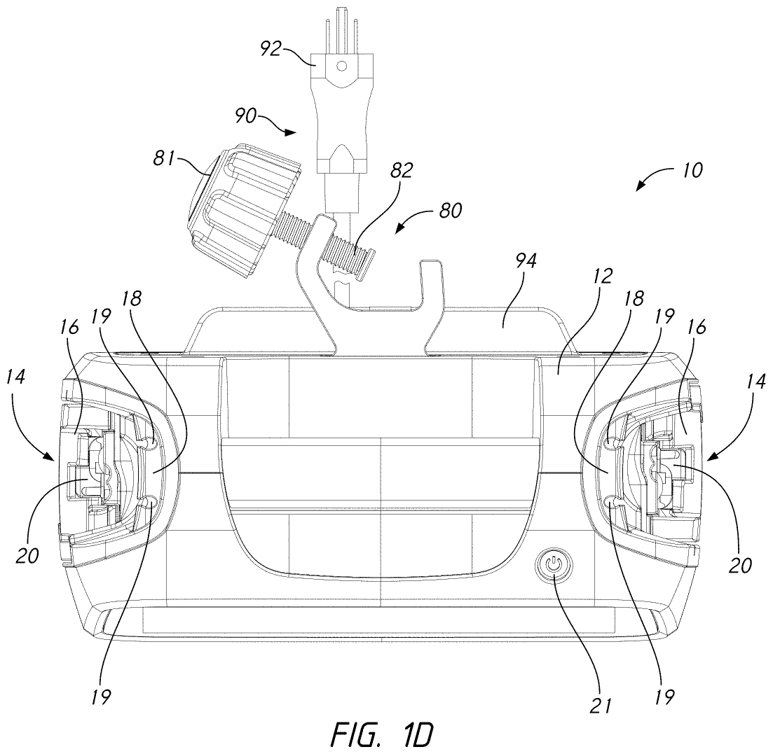

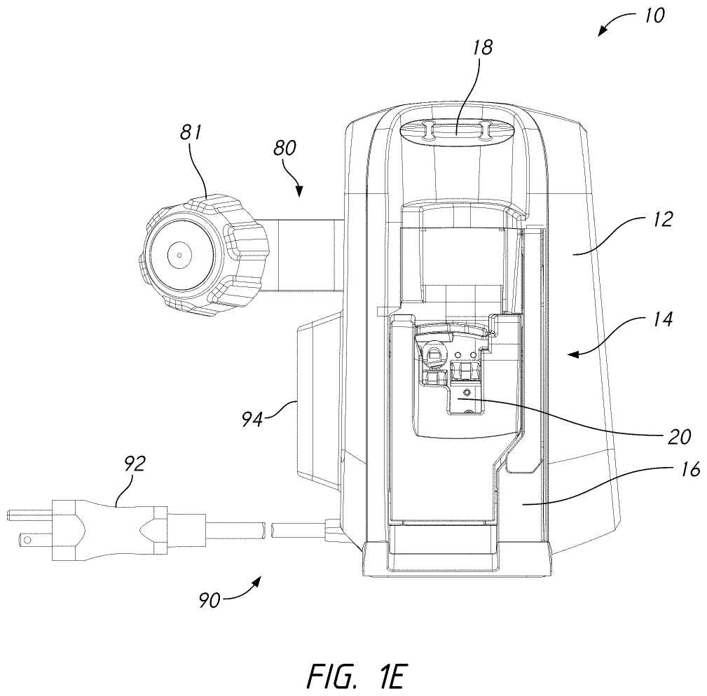

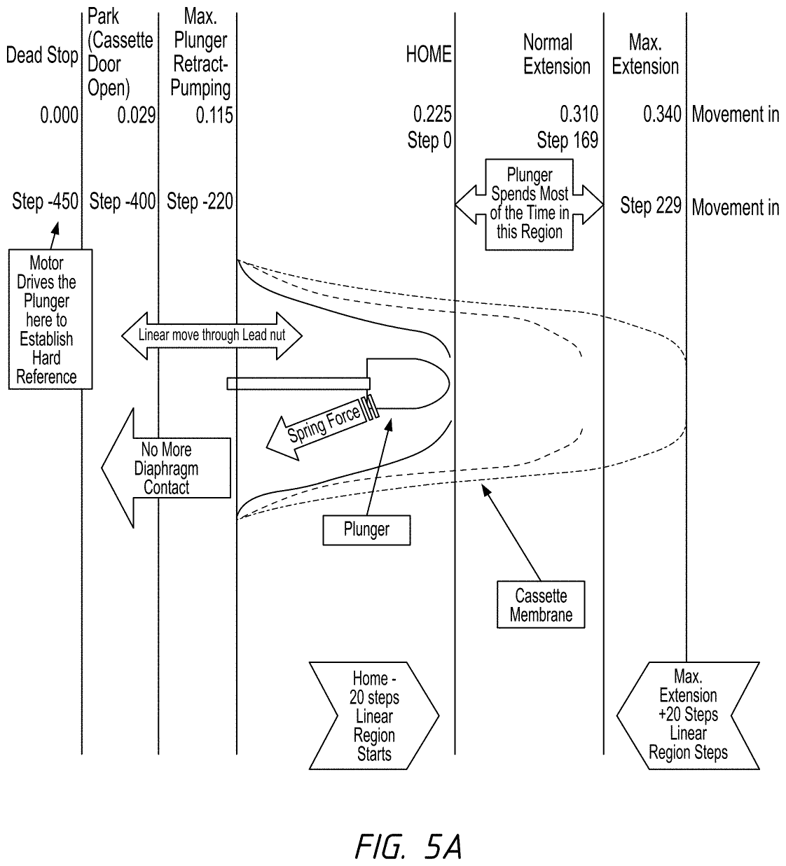

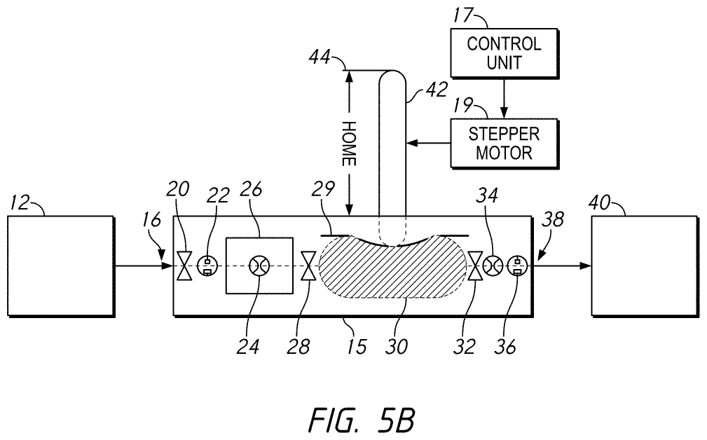

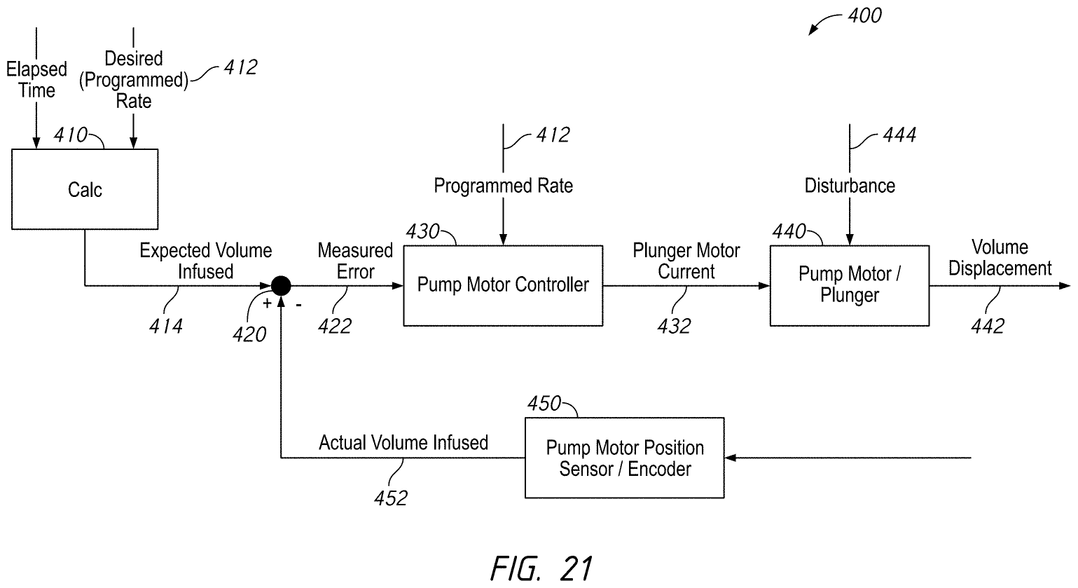

The entire disclosure of U.S. Pat. No. 7,258,534 is incorporated by reference herein, for all purposes, for all that it contains. FIG. 1 of that patent shows a screen that can provide a graphic user interface for communicating medication arrival predictions and the other information disclosed herein. FIG. 5 shows a perspective view of a cassette for a pumping system. FIG. 21 shows a schematic diagram of a pumping system that has sensors. A system with this kind of internal feedback can be especially aware of the parameters that will affect arrival of an infusate. Sensors can contribute for a systems ability to provide precise, predictive, and even prescriptive information to a clinician, as disclosed herein. Column 4 explains that fluid can flow in a series of spaced-apart pulses rather than in a smoothly continuous flow. The disclosure of this patent provides further examples of the type of device-specific information that a pump can obtain, calculate, and provide. A pump can use information from its own sensors, encoders, controllers, motors, and processing unit(s) to predict medication arrival times, for example.

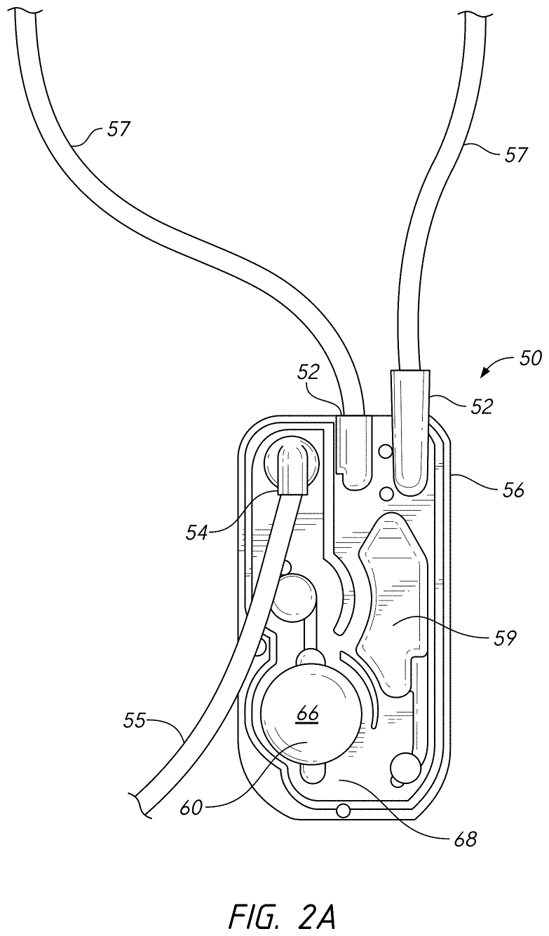

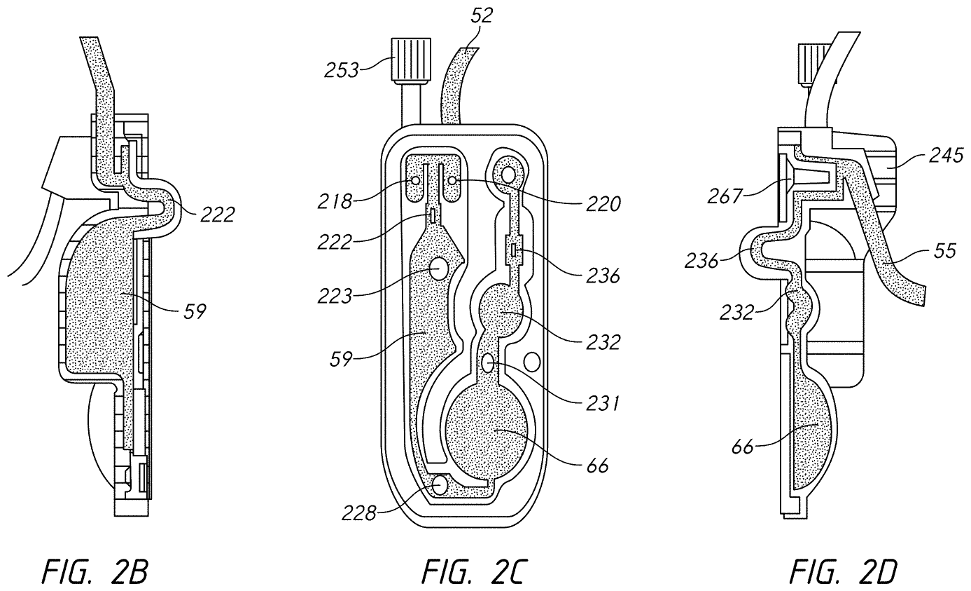

Turning to the figures of the present disclosure, FIGS. 1A-1E show an electronic medical intravenous pump 10 with a housing 12 and at least one pump driver 14 attached to the housing 12. As illustrated, a plurality of pump drivers 14 (e.g., at least two) can be integrally provided within the same housing 12 of a single medical pump 10. Either or both of the pump drivers 14 can include a cover 16 that partially or entirely encloses an outer surface of the pump driver 14, an indicator 18 (e.g., an illuminating communicator) attached to the cover 16, one or more tube holders 19, and a loader 20 configured to securely receive and releasable hold a disposable fluid holder (see, e.g., FIGS. 2A-2D), including but not limited to a cassette, syringe, and/or tubing. The one or more tube holders 19 can be configured to removably receive and securely hold one or more fluid-conveying tubes extending into or exiting from fluid holder when the fluid holder is received into the loader 20. The indicator 18 can communicate one or more messages to a user, such as by temporarily illuminating in one or more colors. Examples of one or more message include confirming that a pump driver 14 near the indicator is currently active and pumping or that one or more instructions being received from a user will apply to a pump driver 14 near the indicator 18. The loader 20 can be a mechanism with multiple moving parts that opens, closes, expands, contracts, clasps, grasps, releases, and/or couples with the fluid holder to securely hold the fluid holder on or within the pump 10 during fluid pumping into the patient. The loader 20 can be integrated into and positioned on or within the pump 10 near the cover 16 adjacent to the indicator 18.

A user communicator, such as display/input device 200, can be provided to convey information to and/or receive information from a user (e.g., in an interactive manner). As illustrated, the user communicator is a touch screen that is configured to provide information to a user through an illuminated dynamic display and is configured to sense a user's touch to make selections and/or to allow the user to input instructions or data. For example, the display/input device 200 can permit the user to input and see confirmation of the infusion rate, the volume of fluid to be infused (VTBI), the type of drug being infused, the name of the patient, and/or any other useful information. The display/input device 200 can be configured to display one or more pumping parameters on a continuing basis, such as the name of the drug being infused, the infusion rate, the volume that has been infused and/or the volume remaining to be infused, and/or the elapsed time of infusion and/or the time remaining for the programmed course of infusion, etc. As shown, the touch screen can be very large, for example at least about 4 inches×at least about 6 inches, or at least about 6 inches×at least about 8 inches. In the illustrated example, the touch screen fills substantially the entire front surface of the pump 10 (see FIG. 1A), with only a small protective boundary surrounding the touch screen on the front surface. As shown, the touch screen comprises at least about 80% or at least about 90% of the surface area of the front of the pump 10. In some implementations, the front of the touch screen comprises a clear glass or plastic plate that can be attached to the housing 20 in a manner that resists liquid ingress, such as using a water-proof gasket and/or adhesive that can withstand repeated exposure to cleaning and sanitizing agents commonly used in hospitals without significant degradation.

An actuator 21 can be provided separate from the user communicator. The actuator 21 can be configured to receive an input and/or display information to a user. As shown, the actuator 21 is a power button that permits the user to press on the actuator 21 to power up the pump 10. The actuator 21 can illuminated to communicate to the user that the pump 10 is power on. If the power source is running low, the actuator 21 can change the color of illumination to quickly show to a user that a power source needs to be replenished.

In some embodiments, the user communicator, such as a display/input device 200, can alternatively or additionally comprise one or more screens, speakers, lights, haptic vibrators, electronic numerical and/or alphabetic read-outs, keyboards, physical or virtual buttons, capacitive touch sensors, microphones, and/or cameras, etc.

During use, the pump 10 is typically positioned near the patient who is receiving fluid infusion from the pump 10, usually lying in a bed or sitting in a chair. In some embodiments, the pump 10 may be configured to be an ambulatory pump, which will typically include a smaller housing, user communicator, battery, etc., so as to be conveniently transportable on or near a mobile patient. In many implementations, the pump 10 is attached to an IV pole stand (not shown) adjacent to the patient's bed or chair. As shown, the pump 10 can include a connector 80 that is configured to removably attach the pump 10 to the IV pole stand. As shown, the connector 80 can comprise an adjustable clamp with a large, easily graspable user actuator, such as a rotatable knob 81 that can be configured to selectively advance or retract a threaded shaft 82. At an end of the shaft 82 opposite from the knob 81 is a pole-contacting surface that can be rotatably advanced by the user to exert a force against a selected region of the pole, tightly pushing the pole against a rear surface of the pump 10, thereby securely holding the pump 10 in place on the pole during use. The selected region of the pole where the contacting surface of the shaft 82 is coupled can be chosen so as to position the pump 10 at a desired height for convenient and effective pumping and interaction with the patient and user.

The pump 10 can include a power source 90. In some embodiments, the power source can comprise one or more channels for selectively supplying power to the pump 10. For example, as illustrated, the power source 90 can comprise an electrical cable 92 configured to be attached to an electrical outlet and/or a portable, rechargeable battery 94. One or more components of the pump 10 can operate using either or both sources of electrical power. The electrical cable 92 can be configured to supply electrical power to the pump 10 and/or supply electrical power to the battery 94 to recharge or to maintain electrical power in the battery 94.

Inside of the housing 20 of the pump 10, various electrical systems can be provided to control and regulate the pumping of medical fluid by the pump 10 into the patient and/or to communicate with the user and/or one or more other entities. For example, the pump 10 can include a circuit board that includes a user interface controller (UIC) configured to control and interact with a user interface, such as a graphical user interface, that can be displayed on the user communicator or display/input device 200. The pump 10 can include a printed circuit board that includes a pump motor controller (PMC) that controls one or more pump drivers 14. In some embodiments, the PMC is located on a separate circuit board from the UIC and/or the PMC is independent from and separately operable from the UIC, each of the PMC and UIC including different electronic processors capable of concurrent and independent operation. In some embodiments, there are at least two PMC's provided, a separate and independent one for each pump driver 14, capable of concurrent and independent operation from each other. The pump 10 can include a printed circuit board that includes a communications engine (CE) that controls electronic communications between the pump 10 and other entities (aside from the user), such as electronic, wired or wireless, communication with a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc. The CE can include or can be in electronic communication with an electronic transmitter, receiver, and/or transceiver capable of transmitting and/or receiving electronic information by wire or wirelessly (e.g., by Wi-Fi, Bluetooth, cellular signal, etc.). In some embodiments, the CE is located on a separate circuit board from either or both of the UIC and/or the PMC(s), and/or the CE is independent from and separately operable from either or both of the UIC and/or the PMC(s), each of the PMC(s), UIC, and CE including different electronic processors capable of concurrent and independent operation. In some embodiments, any, some, or all of the UIC, CE, and PMC(s) are capable of operational isolation from any, some, or all of the others such that it or they can turn off, stop working, encounter an error or enter a failure mode, and/or reset, without operationally affecting and/or without detrimentally affecting the operation of any, some, or all of the others. In such an operationally isolated configuration, any, some, or all of the UIC, CE, and PMC(s) can still be in periodic or continuous data transfer or communication with any, some, or all of the others. The UIC, PMC(s), and/or CE can be configured within the housing 20 of the pump 10 to be in electronic communication with each other, transmitting data and/or instructions between or among each of them as needed.

FIGS. 1A-1E provide example hardware features that can affect when and how fluid is delivered. A display/input device 200 can convey information to a user (e.g., in an interactive manner). For example, it can alert a care provider not to increase a medication rate because, given expected delays, equilibrium or a clinical effect is not yet expected (and a well-intended change in infusion rate to account for apparent lack of effect may actually be detrimental). This and similar scenarios are discussed at greater length elsewhere herein.

FIG. 2A shows an example of a disposable fluid holder, such as a disposable cassette 50, which includes a plastic housing and a flexible, elastomeric silicon membrane. Any structure, material, function, method, or step that is described and/or illustrated in U.S. Pat. No. 4,842,584, which is incorporated herein by reference in its entirety, including but not limited to the pumping cassette, can be used by itself or with or instead of any structure, material, function, method, or step that is described and/or illustrated in this specification. The plastic housing of the cassette 50 can include one or more (e.g., two as shown) fluid inlets 52 and a fluid outlet 54 formed in a main body 56. The cassette 50 can be temporarily positioned for example in the loader 20 of a pump driver 14. The one or more fluid inlets 52 are coupled with one or more inlet tubes 57 in fluid communication with one or more sources of medical fluid, such as one or more IV bags, vials, and/or syringes, etc., containing medical fluid. If multiple inlets 52 and inlet tubes 57 are provided, as shown, then multiple sources of medical fluid can be simultaneously supplied to a patient through the cassette 50. The fluid outlet 54 is coupled to an outlet tube 55 in fluid communication with the patient, normally by way of a needle leading into a patient's blood vessel.

Because the fluid inlets 52 can connect to two incoming (upstream) fluid lines, the example shown in FIG. 2 is configured as a two-line cassette. The fluid path within the cassette and downstream from the cassette will be referred to as a channel.

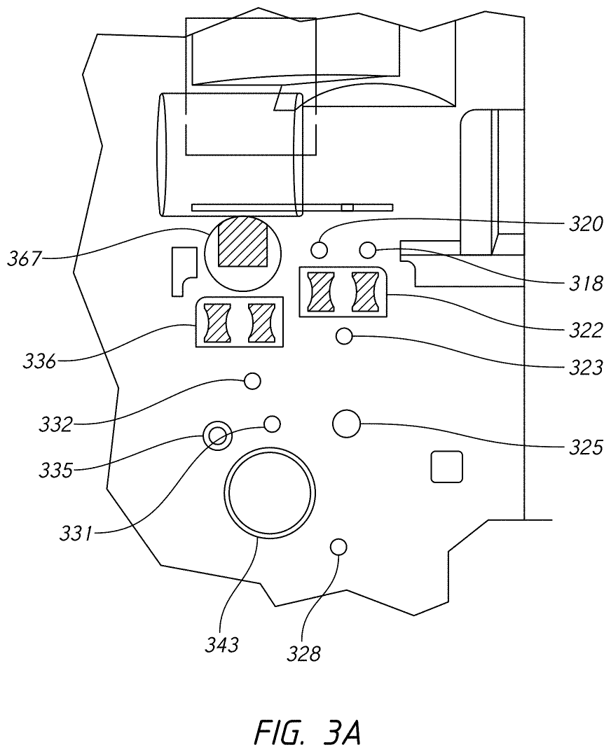

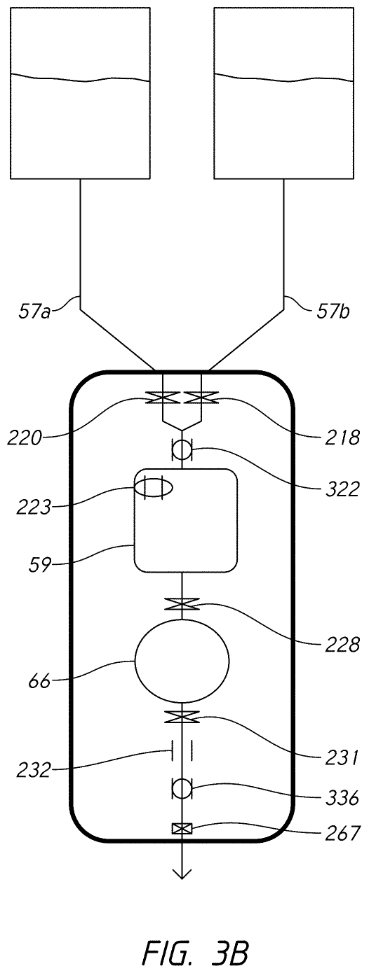

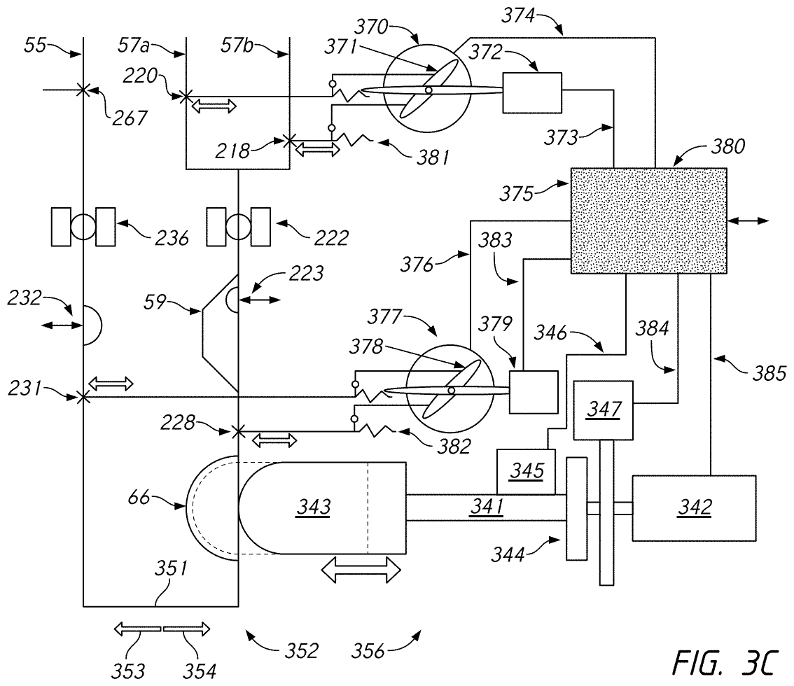

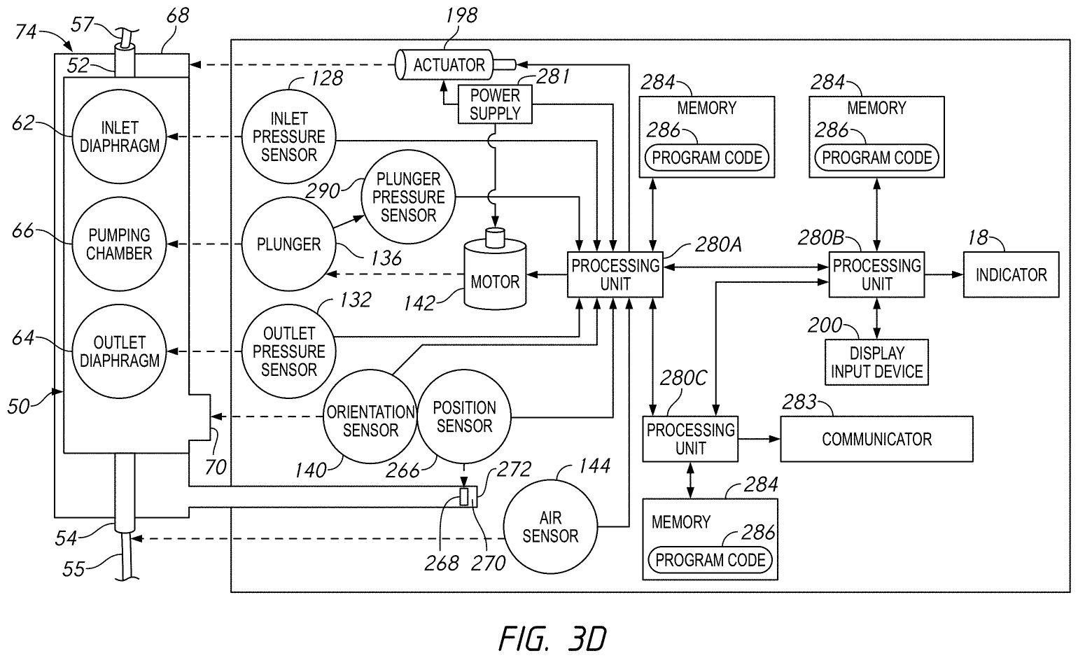

A flexible, elastomeric membrane forms a diaphragm 60 within a pumping chamber 66 on an inner face 68 of the main body 56. In operation, fluid enters through one or more of the inlets 52 and is forced through the outlet 54 under pressure. One or more fluid channels within the main body 56 of the cassette 50 convey the fluid between the inlets 52 and the outlet 54 by way of the pumping chamber 66. Before use, the cassette is typically primed with fluid, usually saline solution. A volume of fluid is delivered to the outlet 54 when a plunger 136 of the pump 10 (see, e.g., FIG. 3) displaces the diaphragm to expel the fluid from the pumping chamber 66. During an intake stroke, the plunger 136 retracts from the diaphragm 60, and the fluid is then drawn in through the inlet 52 and into the pumping chamber 66. In a pumping stroke, the pump 10 displaces the diaphragm 60 of the pumping chamber 66 to force the fluid contained therein through the outlet 54. In some embodiments, the directional movement of flow can be facilitated by one or more directional valve(s) (e.g., at one or more of inlet 52 or outlet 54). The fluid can flow from the cassette 50 in a series of spaced-apart pulses rather than in a smoothly continuous flow. In some embodiments, the pump 10 can deliver fluid to a recipient (e.g., a patient) at a pre-set rate, in a pre-determined manner, and for a particular (e.g., pre-selected) time or total dosage. The cassette 50 can include an air trap 59 in communication with an air vent (not shown).

FIGS. 2B, 2C, and 2D show three views of a cassette similar to FIG. 2A. In FIG. 2C, fluid can flow into an inlet 52, from a primary container, for example. Fluid can also flow into a secondary port 253, which can have a Y-reseal or a locking cap. Fluid coming in from the inlet 52 can pass through an A valve 220. Fluid coming in through a secondary port 253 can pass through a B valve 218. Fluid coming in through these two valves can then pass by a proximal air-in-line sensor 222. Fluid can then pass by, in a widening passage, a proximal pressure sensor 223.

### Cassette Air Trap

The widened passage can form an air trap chamber 59, which can allow for fluid mixing. The air trap chamber is also shown in the side view of FIG. 2B. The air trap chamber 59 can be integral to the cassette. The air trap can be exposed to view above the upper edge of the cassette door when the door is closed. Air passes the proximal air-in-line sensor 222 before entering the air trap, which can have a volume of 2.15 mL. The proximal pressure sensor (see, e.g., pressure sensor 223 of FIG. 3C) can monitor pressure in the air trap chamber 59. In some embodiments, the user can remove air or fluid from the proximal tubing and cassette air trap after the cassette door is closed. To remove air in the trap or the proximal tubing the user may be required to attach a container to a Line B port (e.g., secondary port 253 of FIG. 2C). A control (e.g., on an infuser display screen) can be selected to backprime when a delivery is not in progress. When the user selects backprime, for example, this can initiate rapid pumping of fluid from Line A to a user-attached container on Line B. Advantageously, no fluid is delivered to the cassette distal line during a backprime. After the backprime key is released, a cassette leak test can be automatically performed.

The air-in-line situation, and the backprime remedy, provide an example of the type of information that can be recorded in operation history of an infusion pump, which can then account for such delays in reporting expected infusate arrival times and in-vivo concentrations resulting from fluid infusion.

After passing through an air trap chamber 59, fluid can subsequently flow through an inlet valve 228 and from there into a pumping chamber 66. The pumping chamber 66 is also shown in the side view of FIG. 2D. From the pumping chamber 66, fluid can flow through an outlet valve 231 and then into a widened passage accessed by a distal pressure sensor 232. This passage subsequently narrows down to pass a distal air-in-line sensor 236. The two air-in-line sensors, proximal 222, and distal 236, can both be positioned near a bend in a passage or tubing, as shown in the side views of FIGS. 2B and 2D. Fluid can flow through or pass a precision gravity flow regulator 267, seen in FIG. 2D. A finger grip 245 is also seen protruding to the right in FIG. 2D. An outlet tube 55 is also shown coming from the precision gravity flow regulator 267 and leading to a patient. The features shown in the cross sectional schematics of FIGS. 2B-2D can correspond generally to the external cassette contours shown in FIG. 2A.

A manufacturer of a cassette 50 (or the like) typically has the most information about how its physical hardware works and how it will or will not respond to various situations. For example, to achieve an effective low infusion rate, the hardware may be dormant (the pump may pause) for certain periods and only have intermittent activity. The result of this (and other potential characteristics or features of a given system) is best known to a manufacturer, who can encode the resulting expectations into a memory within the pump itself. Indeed, a pump can be unique not only to a manufacturer, but unique to a particular batch, or even completely unique, when its performance is measured with sufficient accuracy and detail. Accordingly, a pump can carry with it, in its memory, information about its own response times, output volumes, pump mechanism displacement amounts, etc.

### Fluid Delivery

A pumping system or infuser can deliver fluids from one or two drug sources through a sterile fluid pathway of administration set tubing, accessories and a cassette. In some embodiments, there is no contact between the fluid and an infusion mechanism subsystem (see FIG. 3A and the electromechanical portion 356 of FIG. 3C). Single line infusion can, for example, support delivery rates of 0.1-99.9 mL/hr. in 0.1 mL increments, and 100-999 mL/hr in 1 mL increments. The total volume infused can be 0.1-99.9 mL in 0.1 mL increments, or 100-9999 mL in 1 mL increments.

A system user can enter a multi-step therapy program to perform an infusion in a sequence of different delivery rates and volumes. The user can also enter a piggyback therapy program that sequentially delivers fluid from Line B and Line A. Line B starts delivering first and after Line B completes delivery, then Line A delivery is automatically started.

Alternatively, fluid from lines A and B can be interspersed or delivered simultaneously but at different rates such that a consistent ratio is maintained between the substances. For example, a concurrent therapy program can combine fluid from both Line A and Line B in the cassette pumping chamber during each chamber fill cycle, then delivers a combination of the two fluids with each plunger stroke. Such a program can have a 0.5 mL/hr minimum rate for each line. The maximum total rate for both lines combined in a concurrent delivery can be 500 mL/hr.

After the volume to be infused (VTBI) is completed for a delivery, the therapy program can include a keep vein open (KVO) delivery rate. KVO rates can be can be, for example, 1.0 mL/hr, or the last primary delivery rate, etc. In some embodiments, the KVO rate can be configurable, for example, between 0.1 and 20 mL/hr.

A pump system or infuser can be designed and manufactured to maintain a volumetric delivery rate error of the total fluid delivered of less than or equal to ±5% over the course of 48 hours at a programmed rate of 1 to 999 mL/hr. For some pumps, rates below 1 mL/hr, the delivery rate error can be less than or equal to ±5%, but other pumps can have a delivery rate error of less than or equal to ±10%.

A pump system or infuser can be designed and manufactured to maintain a volumetric delivery rate error of the total fluid delivered of less than or equal to ±5% over the course of 48 hours at a programmed rate of 1 to 999 mL/hr. At rates below 1 mL/hr, the delivery rate error can be less than or equal to ±5%. These accuracies can be maintained for filling head heights of 12 to 24 inches with 0 PSIG back pressure.

Delivery accuracy, particularly at very low flow rates, may potentially be affected by several use conditions, including elevated infuser height, venous hypertension, presence of air in the cassette air trap, I.V. solution viscosity, and I.V. solution temperature.

The viscosity of fluid can affect the accuracy of the delivery rate, as can enteral delivery. For many medical fluids the additional effect on delivery accuracy is less than 5%. System accuracy for enteral fluids is defined only for rates of 1 to 200 mL/hr, with no suspended air in the solution, and when using an ICU Medical Plum enteral set.

The above infusion rates, accuracy limits, and operation details can be recorded in a memory of the pump system itself. A system's inherent limitations or capabilities can give rise to default displays showing that arrival of medication (or achieving desired concentration of medication) will not occur before a certain absolute or elapsed time, for example. However, if a pump system somehow performs outside of a predicted range, the pump can also account for that unusual performance in predicting further outcomes, patient medication status, and/or otherwise displaying information to a user.

An additional or alternative infusion pump cassette is illustrated in FIG. 5 of U.S. Pat. No. 7,402,154. An elastomeric membrane 60 forms an inlet diaphragm 62, an outlet diaphragm generally indicated at 64, and a pumping chamber 66 located between the inlet and outlet diaphragms 62 and 64 on an inner face 68 of the main body 56. In operation, fluid enters through the inlet 52 and is forced through outlet 54 under pressure. The fluid is delivered to the outlet 54 when the plunger 136 of the pump 10 displaces the pumping chamber 66 to expel the fluid. During the intake stroke the plunger 136 releases the pumping chamber 66, and the fluid is then drawn through the inlet 52 and into the pumping chamber 66. In a pumping stroke, the pump 10 displaces the pumping chamber 66 to force the fluid contained therein through the outlet 54. The directional movement of flow can be facilitated by one or more directional valve(s) (e.g., at one or more of inlet 52 or outlet 54). At low rates the flow can be delivered in discrete volumes as the pump 10 displaces the pump chamber in successive steps. Thus, the fluid can flow from the cassette 50 in a series of spaced-apart pulses rather than in a smoothly continuous flow. Typically, this pump can deliver fluid to a recipient (e.g., a patient) at a pre-set rate, in a pre-determined manner, and for a particular (e.g., pre-selected) time or total dosage. A flow stop can be formed as a switch in a main body and protrude from the inner surface 68. This protrusion can form an irregular portion of the inner surface 68 which can be used to align the cassette 50 as well as monitor the orientation of the cassette 50. The flow stop can provide a manual switch for closing and opening the cassette 50 to fluid flow. A rim 72 is located around the outer surface of the main body 56 and adjacent the inner surface 68. The rim 72 can be used to secure the cassette in a fixed position relative to the pump 10 of U.S. Pat. No. 7,402,154.

FIG. 3A illustrates infusion mechanism hardware for interacting with the cassette of FIGS. 2A-2D. The illustrated and described features can interact with a cassette having fluid passages to fill the role of the pump driver 14 shown in FIGS. 1A-1E. In FIG. 3A, a B valve interface 318 can correspond to or interact with a B valve 218 as shown in FIG. 2C. Similarly, an A valve interface 320 can correspond to or interact with an A valve 220. A proximal air-in-line sensor 322 can be located outside of a cartridge and can interact with a loop or bend in at least partially transparent fluid pathway, for example. Thus, the sensor 322 is depicted with two vertical portions that can pinch or otherwise be positioned adjacent to a tube running vertically between them. Below, a proximal pressure sensor interface 323 can interact with a pressure sensor 223. A force-sensing resistor 325 can be used to determine whether a cartridge is in physical contact with the hardware, or a portion of a pump having the hardware, shown in FIG. 3A. In many embodiments, an inlet valve 228 is actively driven and can receive actuation from an inlet valve interface 328. Similarly, an outlet valve interface 331 can interact with an outlet valve 231. A plunger 343 can extend toward and interact with a pumping chamber 66 (see FIGS. 2C and 2D). A cassette locator 335 can be used to provide alignment and registration of physical interacting components when a cassette such as shown in FIGS. 2A-2D is inserted into or aligned with the hardware components shown in FIG. 3A. A distal pressure sensor interface 332 is located below a distal air-in-line sensor 336. Above this is located a regulator actuator 367, which can be configured to interact with the precision gravity flow regulator 267.

FIG. 3B illustrates a fluid path through a cassette such as those shown in FIGS. 2A-2D, as controlled by the hardware of FIG. 3A. The physical components of FIGS. 2A-2D and FIG. 3A can control and evaluate fluid in the path illustrated in FIG. 3B. Thus, in FIG. 3B, fluid coming in from either a primary line 57A or a secondary line 57B can pass through the A valve 220 or the B valve 218, respectively. Incoming fluid can then mix in a joined passage, and pass a by a proximal air-in-line sensor 322. Fluid can then enter an air trap chamber 59 having a proximal pressure sensor 223. This chamber can allow fluid from two sources to mix. From here, fluid can flow through an inlet valve 228 and from there into a pumping chamber 66. From the pumping chamber 66, fluid can flow through an outlet valve 231, past a distal pressure sensor 232, and past a distal air-in-line sensor 336. Fluid can flow through or pass a precision gravity flow regulator 267 before proceeding from a cassette toward a patient through tubing.

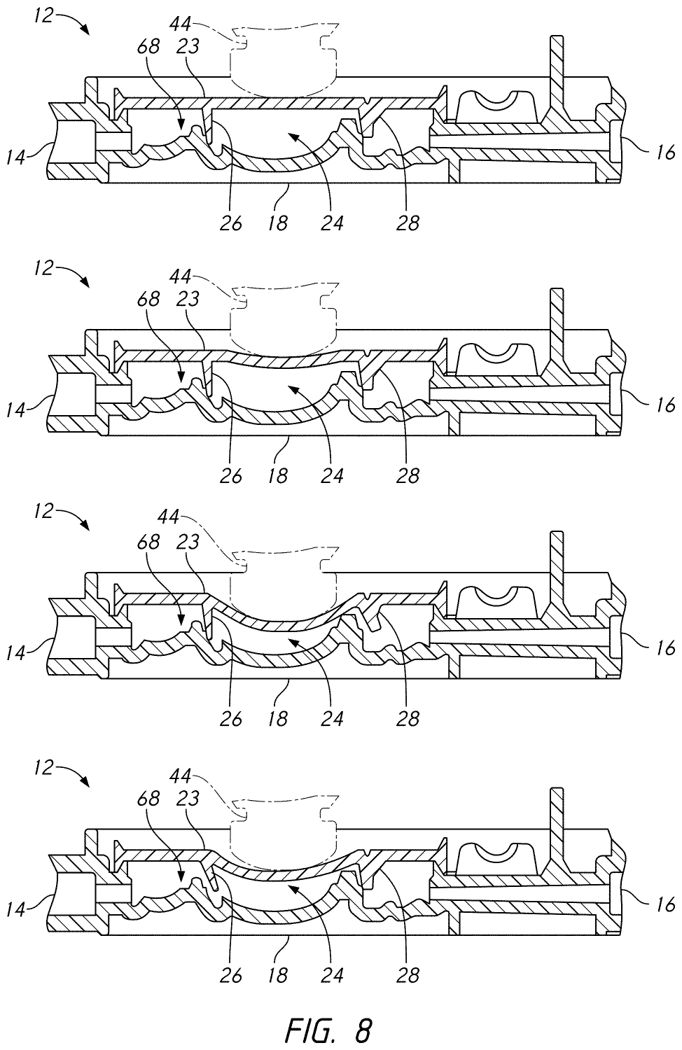

Referring again to FIG. 3B, the volume of a cassette pumping chamber (66) can be controlled by the Infusion Mechanism Subsystem plunger motor (342 in FIG. 3C). After mechanism initialization, the plunger (e.g., 343 in FIGS. 3A, 3C, 5A) presses against the membrane of the pumping chamber (66) and is in the home position (see FIG. 5A). During each pump stroke the plunger pressurizes the pumping chamber by extending the membrane in the direction of a rigid rear (e.g., hemispherical) wall of the pumping chamber (see, e.g., the cross section views of FIG. 8). In some embodiments, the volume of fluid delivered with each full plunger stroke is typically 337 mcl and the plunger extension is 169 steps of the motor from the plunger home position (see FIG. 5A). When the cassette door is opened, the plunger can move to the park position so that it is not in contact with the cassette membrane when the cassette is removed by the user.

In a system using active, positively-controlled valves with motors, during fluid delivery, the plunger (e.g., 343 in FIGS. 3A, 3C, 5A) can repeatedly cycle between the home position and the extended position. To draw fluid into the pumping chamber (e.g., 66) the inlet valve (e.g., 228) is opened. The outlet valve can then promptly close. In some embodiments, opening of the inlet valve can automatically causes the outlet valve (e.g., 231) to close. This causation can occur, for example, if both are mechanically linked—e.g., by the same valve motor drive train (see 377, 378, 382, and 379 of FIG. 3C, for example). The Line A and Line B valves (e.g., 218, 220) control which fluid source is used for all or part of the plunger retraction when fluid is drawn into the pumping chamber (e.g., 66) by the negative pressure. When the plunger reaches the home position the plunger motion pauses while the inlet valve (e.g., 228) is closed, pressure is equalized, and the outlet valve (e.g., 231) is opened. Then the plunger extends and the positive pressure forces fluid out of the pumping chamber and into the distal line (e.g., 55) of the set, which can be connected to a patient.

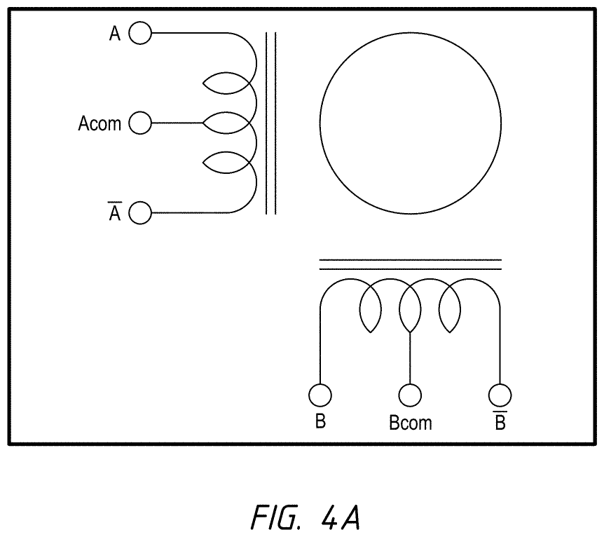

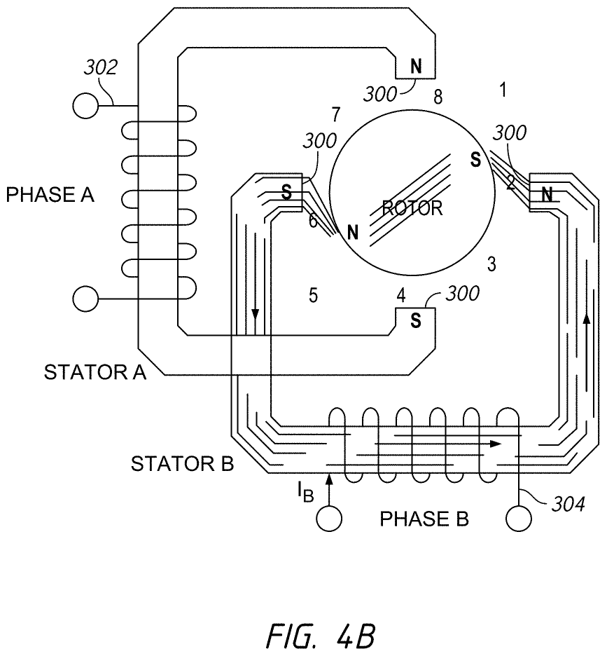

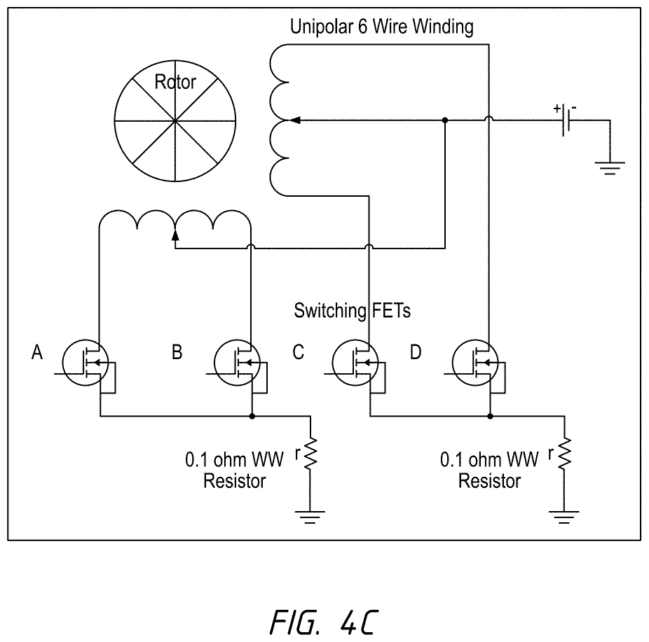

The plunger stepper motor (e.g., motor 342 of FIG. 3C or the motor of FIG. 4C) can be activated by current pulses through the motor windings. In some embodiments, a plunger motor can use different patterns (e.g., 6 different patterns) of pulses can be used, depending on the delivery rate. As the rate increases, a pause between successive steps of the motor decreases. In some embodiments, valve motors can use a single pattern of current pulses through the motor windings. The patterns of current pulses for the motors are advantageously controlled by a PMC microcontroller (e.g., in the controller 380).

The pulse patterns, and any resulting pauses in hardware components (and the effects these patterns and pauses will have on fluid flow) provide an example of the type of information that can be recorded in operation history of an infusion pump, which can then account for such delays in reporting expected infusate arrival times and in-vivo concentrations resulting from fluid infusion. The principles described herein with respect to discrete steps (e.g., at low infusion rates where pump pauses can be easily measured) also apply to more smoothly continuous fluid delivery at a higher rate (e.g., without pauses, or where pulses are so frequent that any pauses are not discernible). Medication levels in a patient are affected by pump operation history, including how motors, valves, and pumping components are designed, actuated, driven, and measured.

FIG. 3C further illustrates schematically how hardware (e.g., FIG. 3A) can interact with a cassette (e.g., FIGS. 2A-2D) along a fluid path. FIG. 3C shows a patient or distal line 55 at the top left corner. Generally at the left is shown a consumable or cassette portion 352. Generally at the right is shown an electromechanical portion 356. In the cassette 352, a distal side 353 is toward the left, and a proximal side 354 is toward the right. A fluid path 351 is illustrated, passing generally from inlets 57A and 57B to outlet 55. Thus, the features illustrated in the above figures are largely present here again at the left hand side of FIG. 3C. Line A 57a leads to a Line A valve or pin 220, which can move to the right and left as shown by the arrow. Similarly, Line B 57B can lead to a Line B valve or pin 218. A spring such as the spring 381 can be deployed with respect to both the valve 218 and the valve 220, and a cam 371 can connect a stepper motor 370 with the valve to 220 and the valve 218. The stepper motor 370 can interact with a line AB position sensor 372, with feedback 373 provided to a controller or controllers 380. A controller 380 can in turn provide input and/or power 374 to the stepper motor 370. In this arrangement, the valves 220 and 218 are actively and positively controlled by a motor and a controller.

For the outlet valve and pin 231 and the inlet valve and pin 228, a stepper motor 377 having a cam 378 and associated springs 382 can interact with the valves 228 and 231. In some embodiments, the cam 371 can cause the associated valves 220, 218 to not be open simultaneously. In some embodiments, the inlet valves 220 and 218 are not open simultaneously to that fluid does not mix in either of inlet lines 57a or 57b.

Similarly for the cam 378 and the valves 231 and 228, if the cam forms a rigid elongate structure as shown, it can pull on one valve while pushing on the other and when it swings the other direction push and pull in an alternating manner. The valves 228 and 231 can open at alternating times such that fluid intake occurs during a draw portion of a plunger stroke, and fluid is expelled during a push portion of a plunger stroke. Having the valve open simultaneously or other synchronization problems can be avoided to discourage backflow.

An input output valve position sensor 379 can be connected to a physical component of the stepper motor 377. The sensor 379 can provide feedback to the controller or controllers 380, which can in turn send input and/or power 376 to the stepper motor 377.

The controller or controllers 380 can also interact with a third stepper motor 342, which can cause movement of a lead screw 341 connected to a plunger or piston 343, which in turn physically interacts with the pumping chamber 66. A linear position sensor 345 can provide feedback 346 of this process to a controller 380. Similarly, a rotary position sensor 347 can provide feedback 384 to a controller 380. Thus, linear and rotary position feedback can be provided either as a backup, as an alternative, or otherwise. A coupler 344 can be provided between the stepper motor of 342 and the lead screw 341. Input and/or power 385 can be provided from the controller 380 to the stepper motor 342. The plunger or piston 343 can follow a reciprocating pattern as shown by the arrow. Thus, the electromechanical portion 356 of a pump can have multiple reciprocating portions and multiple motors. The reciprocation of the valves 220, 218, 231 and 228 can be timed and coordinated with the reciprocation of the piston 343 (e.g., by controller/s 380) to encourage fluid to move through the fluid path 351. Although additional feedback lines are not shown in FIG. 3C, sensor feedback can be provided from the distal air inline sensor 236 and the proximal area line sensor 222, as well as the distal pressure sensor 232 and the proximal pressure sensor 223.

### Valve Operation

In some modes of operation, the valves 218 and 220 can each be open for some percentage of the duration of an intake stroke of the plunger 343, while the inlet valve 228 is open for approximately the entire duration of the same intake stroke. (Concurrent flow can independently control two rates, drawing a proportional amount of fluid from each of lines A and B into the pumping chamber). During an expelling stroke, the outlet valve 231 can remain open approximately the entire time. Intake and expelling strokes can have similar durations. However, an advantageous approach uses a quick intake stroke during which the pump chamber fills, and then a series of smaller output strokes. For example, intake may occur within seconds, while the output strokes continue over a much longer time until the pump chamber needs to be filled again. Proper cadence and sequencing of the motors can be confirmed directly by the feedback from the motors 373, 383, and 385. Proper pressure response of the fluid can be confirmed or measured by the sensors 223 and 232. Potential air bubbles can be evaluated by sensors 222 and 236. System interpretation of sensors 223 and 232, and of 222 and 236, can lead respectively to occlusion alarm and air alarm states that result in unexpected flow discontinuities.

Valve motors such as the motors 370 and 377 of FIG. 3C can be controlled by a pump mechanism controller (“PMC”) microcontroller using a chopper motor drive. The valve motors 370 and 377 can be the same, with one motor used for a pair of valves.

An Inlet/Outlet (I/O) valve motor (e.g., 377 in FIG. 3C) opens and closes the cassette pumping chamber inlet and outlet valves (e.g., 228, 231) in an administration set cassette. The cassette can have a membrane that is exposed by openings in the back of the cassette body above where there are valve chambers in the cassette. The Inlet valve pin (e.g., 228) is opened to allow fluid to enter the pumping chamber (e.g., 66) through the air trap (e.g., 59) from the proximal line, which is selected by the Line A/B Select valves (e.g., 218, 220). When the pumping chamber is filled the Inlet valve (e.g., 228) is closed, the pumping chamber pressure is set and the Outlet valve (e.g., 231) is opened to allow fluid to be pumped into the distal line of the set.

A state machine (e.g., in or associated with the controller 380) can run a program for controlling the I/O valve motor (e.g., 370, 377). In an optical approach, cam flags can protrude from a portion of the drive train. Rotational cam flag signals can be acquired optically during or after each motor step and are monitored using a state machine. As with the other motors, if there is an error in the Inlet/Outlet valve motor position (phase loss), then the motor can be re-initialized to the current position.

The Line A/B Select (LS) valve motor (e.g., 370 in FIG. 3C) opens and closes the Line A and Line B select valves (e.g., 220, 218) in the administration set cassette, using openings in the back of the cassette body for actuator access. The Line A valve (e.g., 220) controls the primary inlet port to the cassette which can be attached permanently to the set proximal tubing. The Line B valve (e.g., 218) controls the secondary inlet port, which may have a screw cap, a Pre-pierced or a Clave attached to it, depending on the type of set.

### Example System Operation

In some embodiments, a pump system can have a cassette door with a handle that supports an administration set cassette such as that illustrated in FIGS. 2A-2D. When the door is open in a loading position the user can slide the cassette into a slot with a cassette guide spring. When the door is closed the cassette is aligned and the front of the cassette makes contact with a door datum surface, actuator and sensor subassemblies (plunger 343 and pins or valves 218, 220, 228, 231) make contact with a cassette elastomeric membrane, and a cassette guide spring can push a fluid shield against the front face of a mechanism chassis. The door can be released from the handle when it is in the loading position, allowing the door to be perpendicular to the mechanism fluid shield. This allows the user to clean the rear of the door and the fluid shield, or to remove any object which has fallen behind the door.

A cassette locator (see, e.g., 335 in FIG. 3A) can be a pin that helps align the cassette with the mechanism as the door is closed and keeps the cassette in the correct position during delivery.

The cassette can have a flow regulator valve (e.g., the precision gravity flow regulator 267, seen in FIG. 2D) distal to the pumping chamber (e.g., the chamber 66 of FIGS. 2A-3D). The flow regulator valve can be closed by the user after an administration set is primed. The proximal line can be clamped as an additional prevention of free flow. As the door is closed, an actuator connected to the door handle can automatically open the flow regulator valve after the pumping chamber outlet valve pin closes the outlet valve. The flow regulator valve can be used by the operator to control fluid flow rate when the administration set is used independently for a gravity drip infusion.

A reciprocating pumping piston/plunger (e.g., the plunger 343 of FIG. 3C) can be actuated by a motor (e.g., the motor 342). As schematically shown in FIG. 3C, a pump plunger motor and drive train can be perpendicular to a pumping chamber membrane opening on the rear of a cassette. The drive train can have location sensors that are monitored by motor control software on a PMC microcontroller (see controller 380 of FIG. 3C). The software can implement state machines which control the motor operation.

An inlet valve to the pumping chamber (e.g., the valve 228) can be actuated by a motor (e.g., the motor 377), and a drive train can extend an actuator through an opening in the rear of the cassette to reach the valve. The same motor can be used for the outlet valve, which can improve synchronization. A default position is with the inlet valve (e.g., the valve 228) closed by a spring (e.g., 382) which can apply steady pressure to a valve pin. The drive train (see generally 377, 378 and related structures) has a location sensor (e.g., 379) that is monitored by (383) motor control software on the PMC microcontroller (e.g., 380). The software implements state machines which can control the motor operation. The same description here generally applies to an outlet valve (e.g., 231), actuated by the same motor (e.g., 377).

Line A select valve (e.g., 220) for primary proximal fluid line A (e.g., 57a) and Line B select valve (e.g., 218) for fluid line B (e.g., 57b) can be actuated by a motor (e.g., 370). As described above for the valves 228 and 231, the valves 220 and 218 can be accessed by a drive train (which may include the cam 371 and springs such as 381) through openings in a cassette, driven by a motor (e.g., 370), as tracked by a location sensor (e.g., 372) and monitored (373) by software in a controller (380).

Proximal and distal air-in-line sensors (222, 236) can be used to detect air passage into (proximal) or out of (distal) the cassette. Both sensors can be ultrasound piezoelectric crystal transmitter/receiver pairs. Liquid in the cassette between the transmitter and receiver conducts the ultrasonic signal, while air does not. This can result in a signal change indicating a bubble in the line.

Proximal and distal MEMS pressure sensors (223, 232 of FIG. 3C) can be used to detect the pressure of the tubing into (proximal) or out of (distal) the cassette. Microelectromechanical systems (MEMS) pressure sensors are an integrated circuit, which have piezo electric resistors diffused into a micro-machined diaphragm to measure strain from a steel ball that extends through the top of the IC package. The steel ball is driven by a pressure pin which is in contact with the cassette membrane.

A cassette presence sensor detects that the cassette is in the door when it is closed. The sensor can be a dome switch mounted in an infusion mechanism subsystem fluid shield. The dome switch can make contact with the cassette when the cassette is correctly aligned with the fluid shield. The switch output signal can be acquired and processed by PMC microcontroller software (e.g., in controller 380).

Motor control interfaces can provide amplification of control signals output by the PMC microcontroller (e.g., the controller 380). PMC microcontroller software can compute motor winding current values which are converted to analog voltages by a digital-to-analog converter (DAC). The control voltages input to the motor control interface can cause amplifiers to drive the selected motor winding with current modulated by a chopper pulse width modulator controller. Preferably, one motor winding is active at a time.

Sensor interfaces in an infusion mechanism subsystem can convert air-in-line, pressure and motor drive position sensor signals into analog voltage signals. The analog voltages are processed by an analog-to-digital converter (ADC) in the PMC microcontroller which outputs digital values. PMC microcontroller software state machines acquire and process data from the sensors.

Non-volatile memory in an infusion mechanism subsystem can be connected to the PMC microcontroller with a serial communications link (SPI bus). The non-volatile memory can be used to store calibration values for the motor drive trains and sensors during manufacturing. Additional system parameters and an alarm log are also stored by the PMC microcontroller in this memory.

The above control and feedback systems generate highly specific, real-time data on how an infusion pump is operating and how fluid in a cassette is responding. This data already exists for precision operation of an infusion device, and it can be conveniently organized and stored (e.g., in a memory of the pump system itself). This data can provide highly accurate predictions of how and when medication will reach a target destination, or achieve a particular level in a target destination. Thus, the sensors, controllers, cam flags, feedback software, etc. described herein is highly valuable in predicting further outcomes, patient medication status, and/or otherwise displaying information to a user.

FIG. 3D is a schematic diagram of functional components for a medical pump (e.g., the pump 10 of FIGS. 1A-1E) used in connection with the disposable cassette 50 (see FIGS. 2A-D) for delivering a fluid to a patient. (Although not shown in this schematic diagram, inlet and outlet valves can alternatively be actively controlled, consistent with the cassette 50 of FIGS. 2A-D, and an upstream air sensor can be provided). One or more processors or processing units 280 can be included in pump 10 that can perform various operations, some examples of which are described in greater detail below. The processing unit(s) 280 and all other electrical components within the pump 10 can be powered by a power supply 281, such as one or more components of power source 90 of pump 10. In some embodiments, the processing unit 280a can be configured as a pump motor controller (PMC) to control the electric motor 142 being energized by the power supply 281. When energized, the electric motor 142 can cause the plunger 136 to reciprocate back and forth to periodically actuate, press inward, and/or down-stroke, causing plunger 136 to temporarily press on pumping chamber 66, driving fluid through cassette 50. The motor 142, plunger 136, sensors 128, 290, 132, 140, 266, 144 can be included in or as an integrated part of the pump driver 14 of the pump 10. In some embodiments, as shown, the inlet pressure sensor 128 engages the inlet diaphragm 62 of cassette 50, and the outlet pressure sensor 132 engages the outlet diaphragm 64 of cassette 50. When retracting, moving outward, or on an up-stroke, the plunger 136 can release pressure from pumping chamber 66 and thereby draw fluid from inlet 52 into pumping chamber 66. Differential pressure within the cassette thus drives the inlet opening during the pump chamber fill cycle. (This is a passive valve approach; it is different from an active valve control). In some implementations of cassette 50, a flow stop 70 is formed as a pivotal switch in the main body 56 and protrudes a given height from the inner surface 68. This protrusion forms an irregular portion of the inner surface 68 which can be used in some embodiments to align the cassette 50 as well as monitor the orientation of the cassette 50. In some embodiments, one form of a flow stop 70 can provide a manual switch or valve for closing and opening the cassette 50 to fluid flow.

In some embodiments, the processing unit 280a can control a loader 20 of the pump 10 with an electronic actuator 198 and a front carriage being energized by the power supply 281. When energized, the actuator 198 can drive the front carriage 74 between closed or open positions. The front carriage 74 in the open position can be configured to receive the cassette 50 and in the closed position can be configured to temporarily securely retain the cassette 50 until the front carriage is moved to the closed position. A position sensor 266 for the cassette 50 can be provided in the pump 10. The position sensor 266 can monitor the position of a slot 268 formed in a position plate 270. The position sensor 266 can monitor a position of an edge 272 of a position plate 270 within the pump 10. By monitoring the position of the position plate 270, the position sensor 266 can detect the overall position of the front carriage of the loader 20. The position sensor 266 can be a linear pixel array sensor that continuously tracks the position of the slot 268. Of course, any other devices can be used for the position sensor 266, such as an opto-tachometer sensor.

A memory 284 can communicate with the processing unit 280a and can store program code 286 and data necessary or helpful for the processing unit 280 to receive, determine, calculate, and/or output the operating conditions of pump 10. The processing unit 280a retrieves the program code 286 from memory 284 and applies it to the data received from various sensors and devices of pump 10. The memory 284 and/or program code 286 can be included within or integrally attached to (e.g., on the same circuit board) as the processing unit 280a, which in some embodiments can be the configuration for any processor or processing unit 280 in this specification.

In some embodiments, the program code 286 can control the pump 10 and/or track a history of pump 10 operation details (which may be recorded and/or otherwise affected or modified, e.g., in part by input from sensors such as air sensor 144, position sensor 266, orientation sensor 140, outlet pressure sensor 132, plunger pressure sensor 290, inlet pressure sensor 128, etc.) and store and/or retrieve those details in the memory 284. The program code 286 can use any one or more of these sensors to help identify or diagnose pumping problems, such as air in a pumping line, a pumping obstruction, an empty fluid source, and/or calculate expected infusate arrival time in a patient. The display/input device 200 can receive information from a user regarding a patient, one or more drugs to be infused, and details about a course of infusion into a patient. The display/input device 200 can provide a clinician with any useful information regarding the pumping therapy, such as pumping parameters (e.g., VTBI, remaining volume, infusion rate, time for infusion, elapsed time of infusion, expected infusate arrival time, and/or time for completion of infusion, etc.) Some or all of the information displayed by the display/input device 200 can be based on the operation details and calculations performed by the program code 286. The program code 286 can use the details stored in the memory 284 to calculate expected infusate arrival time. A display/input device 200 can provide a clinician with selected infusate delivery rate and expected infusate arrival time. This can be based on the operation details and calculations performed by the program code 286.

In some embodiments, the operation details can include information determined by the processing unit 280a. The processing unit 280a can process the data from pump 10 to determine some or all of the following operating conditions: whether or when the cassette 50 has been inserted, whether or when the cassette 50 is correctly oriented, whether or when the cassette 50 is not fully seated to the fixed seat 162, whether or when the front carriage assembly 74 is in an open or closed position, whether or when a jam in the front carriage assembly 74 is detected, whether or when there is proper flow of fluid through the cassette 50 to the patient, and whether or when one or more air bubbles are included in the fluid entering, within, and/or leaving cassette 50. The processing unit 280a can be configured to determine one or more operating conditions to adjust the operation of the pump 10 to address or improve a detected condition. Once the operating condition has been determined, the processing unit 280a can output the operating condition to display 200, activate an indicator window, and/or use the determined operating condition to adjust operation of the pump 10.

For example, the processing unit 280a can receive data from a plunger pressure sensor 290 operatively associated with the plunger 136. The plunger pressure sensor 290 can sense the force on plunger 136 and generate a pressure signal based on this force. The plunger pressure sensor 290 can communicate with the processing unit 280a, sending the pressure signal to the processing unit 280a for use in helping to determine operating conditions of pump 10.

The processing unit 280a can receive an array of one or more items of pressure data sensed from the cassette inner surface 68 determined by the plunger pressure sensor 290 and inlet and outlet pressure sensors 128 and 132. The processing unit 280a can combine the pressure data from the plunger pressure sensor 290 with data from inlet and outlet pressure sensors 128 and 132 to provide a determination as to the correct or incorrect positioning of cassette 50. In normal operation, this array of pressure data falls within an expected range and the processing unit 280a can determine that proper cassette loading has occurred. When the cassette 50 is incorrectly oriented (e.g., backwards or upside down) or when the cassette 50 is not fully seated to the fixed seat 162, one or more parameters or data of the array of pressure data falls outside the expected range and the processing unit 280a determines that improper cassette loading has occurred.

As shown, in some embodiments, the processing unit 280a can receive data from one or more air sensors 144 in communication with outlet tube 55 attached to the cassette outlet 54. An air sensor 144 can be an ultrasonic sensor configured to measure or detect air or an amount of air in or adjacent to the outlet 54 or outlet tube 55. In normal operation, this air content data falls within an expected range, and the processing unit 280a can determine that proper fluid flow is in progress. When the air content data falls outside the expected range, the processing unit 280a can determine that improper air content is being delivered to the patient.

Processing unit 280a can continuously or periodically communicate with an independent and separate processor or processing unit 280b to communicate information to the user and/or to receive data from the user that may affect pumping conditions or parameters. For example, processing unit 280a can communicate by wire or wirelessly with processing unit 280b which can be configured as a user interface processor or controller (UIC) to control the output and input of display/input device 200, including by displaying an operating condition and/or activate indicator 18 to communicate with a user. In some embodiments, processing unit 280b can receive user input regarding pumping conditions or parameters, provide drug library and drug compatibility information, alert a user to a problem or a pumping condition, provide an alarm, provide a message to a user (e.g., instructing a user to check the line or attach more fluid), and/or receive and communication information that modifies or halts operation of the pump 10.

An independent and separate processor or processing unit 280c can be configured as a communications engine (CE) for the pump, a pump communications driver, a pump communications module, and/or a pump communications processor. Processing unit 280c can continuously or periodically communicate with processing units 280a and 280b to transmit and/or receive information to and from electronic sources or destinations separate from, outside of, and/or remote from, the pump 10. As shown, processing unit 280c can be in electronic communication with or include a memory 284 and program code 286, and processing unit 280c can be in communication with and control data flow to and from a communicator 283 which can be configured to communicate, wired or wirelessly, with another electronic entity that it separate from the pump 10, such as a separate or remote user, a server, a hospital electronic medical records system, a remote healthcare provider, a router, another pump, a mobile electronic device, a near field communication (NFC) device such as a radio-frequency identification (RFID) device, and/or a central computer controlling and/or monitoring multiple pumps 10, etc. The communicator 283 can be or can comprise one or more of a wire, a bus, a receiver, a transmitter, a transceiver, a modem, a codec, an antenna, a buffer, a multiplexer, a network interface, a router, and/or a hub, etc. The communicator 283 can communicate with another electronic entity in any suitable manner, such as by wire, short-range wireless protocol (Wi-Fi, Bluetooth, ZigBee, etc.), fiber optic cable, cellular data, satellite transmission, and/or any other appropriate electronic medium.

As shown schematically in FIG. 3, a pump 10 can be provided with many components to accomplish controlled pumping of medical fluid from one or more medical fluid sources to a patient. For example, one or more processors or processing units 280 can receive various data useful for the processing unit(s) 280 to calculate and output the operating conditions of pump 10.

The processing unit(s) 280 can retrieve the program code 286 from memory 284 and apply it to the data received from various sensors and devices of pump 10, and generate output(s). The output(s) are used to communicate to the user by the processing unit 280b, to activate and regulate the pump driver by the processing unit 280a, and to communicate with other electronic devices using processing unit 280c.

Information from one or more of the processing unit(s) 280 can be communicated and displayed on the display/input device 200. Those of ordinary skill in the art will appreciate that display/input device 200 may be provided as a separate display device and a separate input device. Additional or multiple separate display devices and/or multiple separate input devices may be provided. For example, as shown in FIG. 1, the medical pump 10 can include a display/input device 200.

### Example Motors

FIG. 4A illustrates an example motor. Valve motors such as the motors 370 and 377 of FIG. 3C can be four phase stepper types, meaning one electrical revolution is completed after going through a sequence of 4 steps or motor phases. The number of motor poles determines the number of steps per shaft revolution and hence the step-angle. In some embodiments, step angle resolution is 7.5°/step (48 steps per revolution). Electrically, the motor windings can be unipolar, with a center tap connected on each of two coils as shown in FIG. 4A. Unidirectional current enters the center tap, Acom for instance, and is steered to one end of the coil or the other end by the driver electronics, creating positive or negative flux lines in the motor coil. With two coils each with a choice of flux polarity, four electrical combinations or phases are possible. The motors 370 and 377 can operate at constant speed 600 RPM with full step mode.

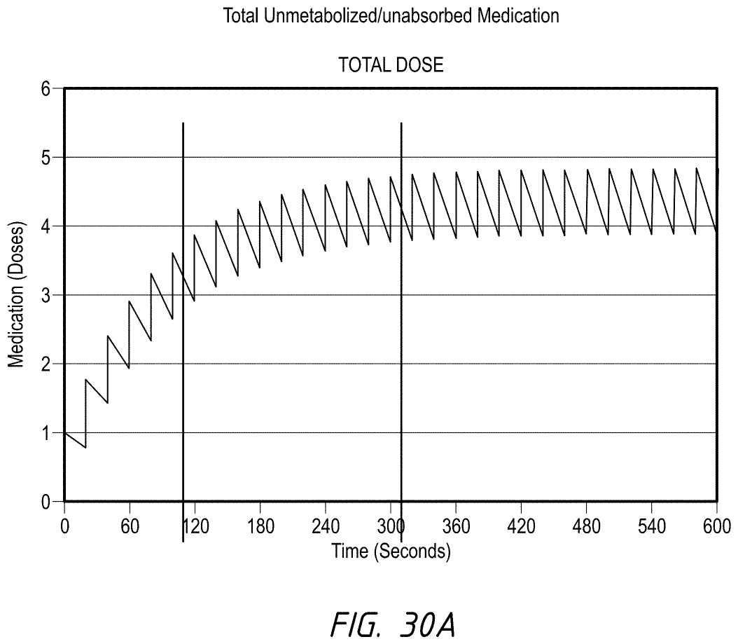

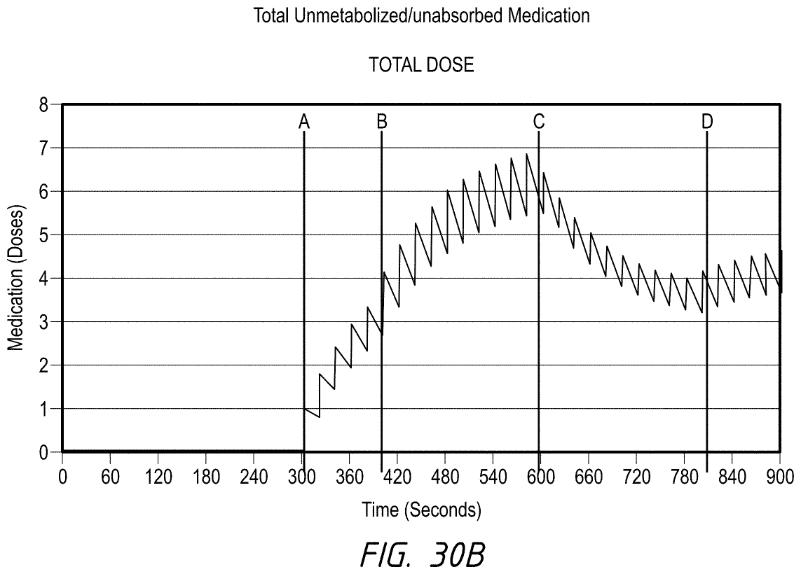

The entire disclosure of U.S. Pat. No. 6,285,155 is incorporated by reference herein, for all purposes, for all that it contains. This patent discloses a pseudo half-step motor drive method that reduces a ringing affect useful in stepper motor control. FIG. 30 of this patent schematically shows how a pump can be positioned between a medicinal fluid and a patient and can employ a control unit and a mechanical pump unit. At columns 6 and 7, this patent explains that a cassette infusion pump can be used for infusing medicinal fluid into a patient's body at very precise flow rates. However, when the associated stepper motors operate a low speeds, a ringing effect can cause problems. At columns 13 and 14, this patent concludes an explanation of how overshoot and ringing can be eliminated. The method(s) disclosed in this patent provide examples of the type of device-specific information that a pump can have stored in its own memory. A pump can account for its own control sequencing to predict medication arrival times, for example. This prediction can be reflected in a graphic user interface for communicating with a clinician.

Stepper motors can be used in a wide variety of devices, including printers, disk drives, and other devices requiring precise positioning of an element. Stepper motors provide many advantages over other types of motors, most notably the ability to rotate through controlled angles of rotation, called steps, based on command pulses from a driver circuit. The accuracy of the stepped motion produced by a stepper motor is generally very good, since there is not a cumulative error from one step to another. The ability to incrementally rotate a shaft through a defined number of fixed steps enables stepper motors to be used with open-loop control schemes (i.e., applications in which a position feedback device such as an optical encoder or resolver is unnecessary), thereby simplifying the motion control system and reducing costs.

The speed of stepping motors can be readily controlled based on the pulse frequency employed, enabling stepping motors to achieve very low speed synchronous movement of a load that is directly coupled to the drive shaft of the motor. Furthermore, stepper motors are reliable, since they often do not include contact brushes that can wear out. Typically, the only parts in a stepper motor susceptible to wear are the motor bearings.

There are three basic types of stepper motor, including a variable-reluctance (VR), a permanent magnet (PM), and a hybrid (HB). A VR stepper motor comprises a soft iron multi-toothed rotor and a wound stator. When the stator windings (also commonly referred to as the motor “coils) are energized with a DC current, a magnetic flux is produced at the poles of the stator. Rotation occurs when the rotor teeth are magnetically attracted to the energized stator poles. PM Stepper motors have permanent magnets added to the motor structure. The rotor no longer has teeth, as in the VR motor. Instead, the rotor includes permanent magnets with the alternating north and south poles disposed in a straight line, parallel to the rotor shaft. These magnetized rotor poles provide an increased magnetic flux intensity, resulting in improved torque characteristics when compared with VR stepper motors.

An HB stepper motor is more expensive than a PM stepper motor, but provides better performance with respect to step resolution, torque and speed. Typical step angles for the HB stepper motor range from 3.6 to 0.9 (100-400 steps per revolution). The HB stepper motor combines the best features of both the PM and VR type stepper motors; its rotor is multi-toothed, like the VR motor, and includes an axially magnetized concentric magnet around its shaft. The teeth on the rotor provide an even better flux path, which helps guide the magnetic flux to preferred locations in the air gap between the rotor and the stator teeth. This configuration further increases the detent, holding, and dynamic torque characteristics of the HB stepper motor, when compared with both the VR and PM stepper motors. Stepper motors generally have two phases, but three, four and five-phase motors also exist.

FIG. 4B shows a two-phase motor, comprising a Stator A and a Stator B, each of which produce a magnetic flux with opposite poles at end faces 300 when a respective phase A winding 302 and phase B winding 304 are energized with an electric current. The direction of the magnetic flux is determinable by applying the “right-hand rule.” In FIG. 4, a current I flows through the phase B windings, creating a magnetic flux in stator B, as indicated by the direction of the arrows. This flux produces a torque applied to the rotor, causing the rotor to turn so that the magnetic field produced by the poles in the rotor are aligned with the magnetic field produced by stators A and B. In this case, the rotor will rotate clockwise so that its south pole aligns with the north pole of stator B at a position 2, and its north pole aligns with the south pole of stator B at a position 6. To continually rotate the rotor, current is applied to the phase A and phase B windings in a predetermined sequence, producing a rotating magnetic flux field.

The output torque of the motor drive shaft is proportional to the intensity of the magnetic flux generated when the winding is energized. The basic relationship determining the intensity of the magnetic flux is defined by

H=(N×i)/l

where N is the number of winding turns, i is the current, H is the magnetic field intensity, and l is the magnetic flux path length. This relationship shows that the magnetic flux intensity, and consequently the torque, is proportional to the number of turns in the winding and the current, and is inversely proportional to the length of the magnetic flux path. In addition, stepper motors that include permanent magnets produce a built-in “detent” torque. This detent torque results from the magnetic flux generated by the permanent magnets, and produces a “cogging” effect that is felt when turning a PM or HB stepper motor that is not energized.

FIG. 4C shows an example pump motor that can be controlled by a PMC microcontroller (e.g., in a controller 380 in FIG. 3C) using a chopper motor drive. A unipolar motor drive can benefit by requiring half as many FET switches as a bipolar motor drive, saving printed circuit board space and cost. Unipolar winding can also be more effective at high speeds because only half of the winding coils are used at a time, which reduces inductance. However, at lower speeds there is less torque because at high speeds the inductance of the motor is the dominant circuit element.

FIG. 5A schematically shows how plunger motor position can change over time to drive fluid through a cassette. Pumping can preferably be performed in a linear region of pumping chamber volume change by extending the plunger 343 from the home (step 0) position to step 169 and then retracting it back to the home position. The home position of the plunger 343 causes the cassette membrane to be partially extended into the pumping chamber (see 66 in FIG. 3C). The park position of the plunger is at step −400 from the home position and it is the location of the plunger after the motor is disabled by the PMC motor control software.

In some embodiments, multiple (e.g., 6) motor control algorithms can be used for single line delivery depending on the delivery rate. The motor control algorithms can use fewer, longer current pulses as the delivery rate increases to allow the CPU to generate the motor control signals rapidly enough as the motor shaft moves faster. The algorithms are designed to reduce acoustic noise and to use less power to preserve battery life. The plunger motor operates from 48 rpm to 305 rpm. From delivery 404 ml/hr to 999 ml/hr, the plunger operates in continuous 1/16 micro-stepping mode. Below 404 ml/hr it operates at discontinuous mode.

The patterns of current pulses, the motor control algorithms, and any resulting effects these patterns and pauses will have on fluid flow provide an example of the type of information that can be recorded in operation history of an infusion pump, which can then account for such delays in reporting expected infusate arrival times and in-vivo concentrations resulting from fluid infusion. The principles described herein with respect to discrete steps (e.g., at low infusion rates where pump pauses can be easily measured) also apply to more smoothly continuous fluid delivery at a higher rate, where pulses are so frequent that they appear to provide continuous delivery (e.g., any pauses are not present or discernible). Medication levels in a patient are affected by pump operation history, including how motors, valves, and pumping components are designed, actuated, driven, and measured.

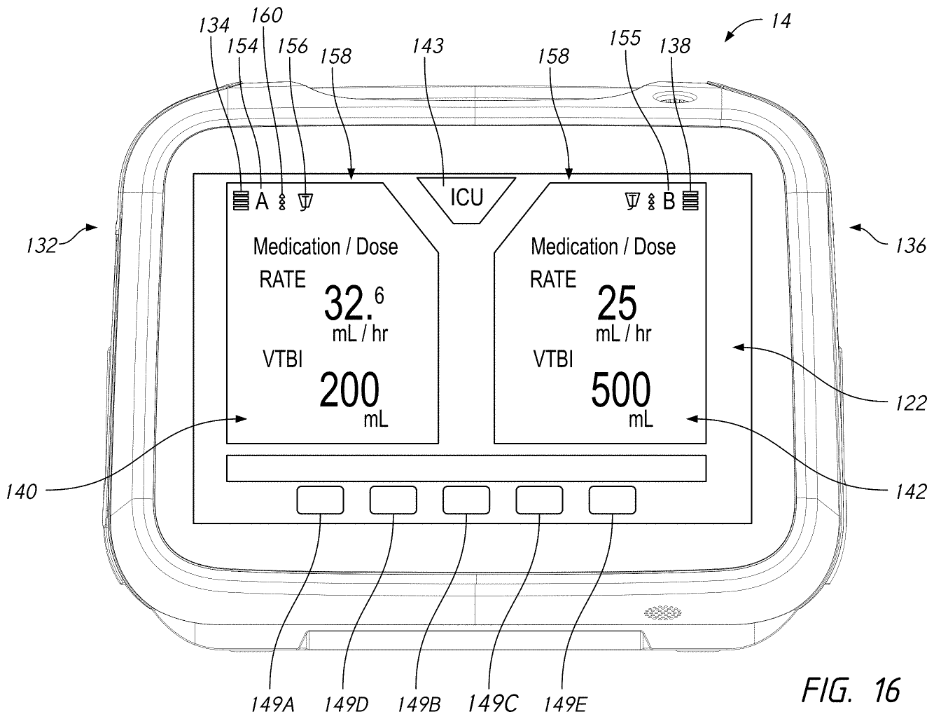

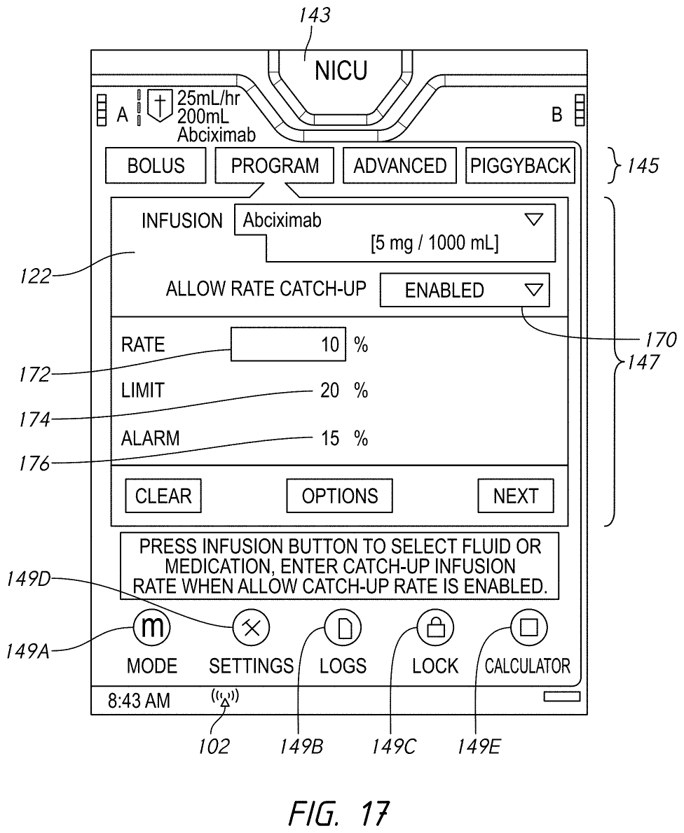

FIG. 5B schematically shows an example of a device that can be driven with a stepper motor. The device is a cassette infusion pump, which is used for infusing medicinal fluid into a patient's body at very precise flow rates. Further details of the device are disclosed in U.S. Pat. No. 6,497,680, the entire specification and drawings of which are hereby specifically incorporated herein by reference, for all that they contain for all purposes. The cassette infusion pump described in that patent can be used for pumping and many of the principles and concepts described herein (e.g., with respect to FIG. 16 and FIG. 17 herein) also have relevance in the context of that pump. Pump Examples

Example pumps that can be used with or demonstrate the principles of the present disclosure include the Plum 360™, available from ICU Medical, Inc. The Plum 360 cartridge supports two (upstream) “lines” or incoming tubes, and one “channel” (within and downstream from the cassette), similar to the arrangement shown in FIG. 2. Thus, the illustrated cartridge is a multi-line cartridge in that two fluid sources (e.g., IV bags) supply fluid through two lines into a single cassette, which then has a single outlet that delivers fluid to the patient. Some pumps can use multiple such cassettes or cartridges, in a multi-line, multi-channel arrangement. For example, an apparatus can have two parallel pumping mechanisms, each corresponding to a separate cartridge, each of which in turn has two incoming lines. In this arrangement, a single device can accept four incoming lines and control two outgoing channels that may ultimately flow to the same patient).

A single channel pump is shown in FIG. 5B for illustrative purposes. In this figure, a source 12 of fluid (e.g., of medicinal fluid) is coupled in fluid communication with a proximal end 16 of a cassette 15. The flow of medicinal fluid into the cassette is selectively controlled by a supply valve 20. After entering a passage in the cassette, the medicinal fluid flows through an air sensor 22 and into a mixing chamber 26. A proximal (or inlet) pressure sensor 24 is disposed adjacent to mixing chamber 26. The fluid exits the mixing chamber through an inlet valve 28, when the inlet valve is in its open position, and flows into a pumping chamber 30. (This can correspond to the pumping chamber 66 of FIGS. 2A-3D, for example). One side of chamber 30 is covered with an elastomeric membrane 29. Fluid is forced from pumping chamber 30 (when inlet valve 28 is closed and an outlet valve 32 is opened), as a plunger 42 (see also the plungers 343 of FIGS. 3A and 3C, and 136 of FIG. 3D) acts on the elastomeric membrane, forcing the elastomeric membrane into the chamber to displace the fluid contained therein. This plunger action is facilitated by positioning a linear drive mechanism, e.g., a lead screw or ball screw (not shown) with a stepper motor 19. In some embodiments, the plunger position is variable from −489 steps to +220 Steps, where a home position is nominally defined to be at 0 Steps. A nominal stroke distance for plunger 42 to deliver 333 μl of fluid is +169 steps.

When outlet valve 32 is in its open position, the fluid forced from the chamber flows past a distal pressure sensor 34, through a distal air sensor 36, and exits the cassette through a tube set, through which it is conveyed to a destination 40. The infusion pump also includes a control unit 17 for the stepper motor. Control unit 17 preferably includes or communicates with at least one microprocessor, a memory, and a motor driver (not separately shown in this figure), which enable execution of a control algorithm for controlling the operation of the infusion pump to deliver the medicinal fluid as desired. The microprocessor controls the stepper motor to control the plunger position, and the plunger forces fluid from chamber 30.

In FIG. 5B, plunger 42 is shown in a home position (at the 0 step position). This position corresponds to the initiation of a pump cycle. Plunger 42 is in contact with the elastic membrane of pumping chamber 30, causing a slight deflection of the membrane. At the beginning of a pump cycle, outlet valve 32 is closed, inlet valve 28 is open, supply valve 20 is in the open position, and pumping chamber 30 is filled with the appropriate amount of fluid.

The use of a stepper motor enables the infusion pump to provide a wide range of delivery rates, making the device especially well suited for use in administering fluid at extremely low medicinal fluid delivery rates. Low infusion rates are particularly useful in pediatric settings. For example, this infusion pump can supply a controlled rate of medicinal fluid at rates as low as 100 μl/hr. This rate is achieved by stepping the stepper motor once approximately every 70 Seconds, so that each step delivers 2 μl of medicinal fluid to the patient.

Information like this—that for this pump, a particular rate (e.g., 100 μl/hr) corresponds to a particular pause duration (e.g., one step every 70 seconds)—can be used by the system to predict when fluid will arrive at an infusing destination. Thus, a correspondence table showing rates and pauses can be stored in the pump or system's memory. This can be incorporated into calculations (along with other inputs—for example, tube length between the pump and a patient) such that the pump or system can display predicted arrival times to a user.

### Obtaining Accurate Tube and Other Information

The entire disclosure of U.S. Patent Publication No. 20200335195 is incorporated by reference herein, for all purposes, for all that it contains. This publication explains how information related to liquid containers and connected tubing can be encoded and transferred efficiently to an infusion pump. For example, the labeling, scanning, and communications options discussed in the '195 patent publication can be used to encode or record information about tubing dimensions (e.g., length, volume, etc.). This information can be accurately and quickly scanned or otherwise transferred to an infusion pump using the systems and methods explained and referred to in the '195 patent publication.

### Example Pump Driving and Compensation

The entire disclosure of U.S. Pat. No. 6,497,680 is incorporated by reference herein, for all purposes, for all that it contains. FIGS. 1 and 2 of that patent show example pump layouts and features that can be used with the present disclosure. This patent addresses problems with delivery accuracy of pumps, especially when inlet and outlet pressures vary substantially, and when delivery rate is low (as in the applications discussed here). Column 2 explains that in cassette pumps, fluid is controlled by varying the number of pumping cycles per unit of time. Column 14 describes a length of time between pump cycle n and pump cycle n+1; a higher medicinal fluid delivery rate requires less time between successive pump cycles. The patent discloses sensors that can provide input for calculating a correction factor that is used to vary pump delivery strokes. These sensors (and resulting correction factors) can provide device-specific information that is also used to further calculate and alert clinicians about what to expect for infusate arrival, equilibrium, or related clinical effects.

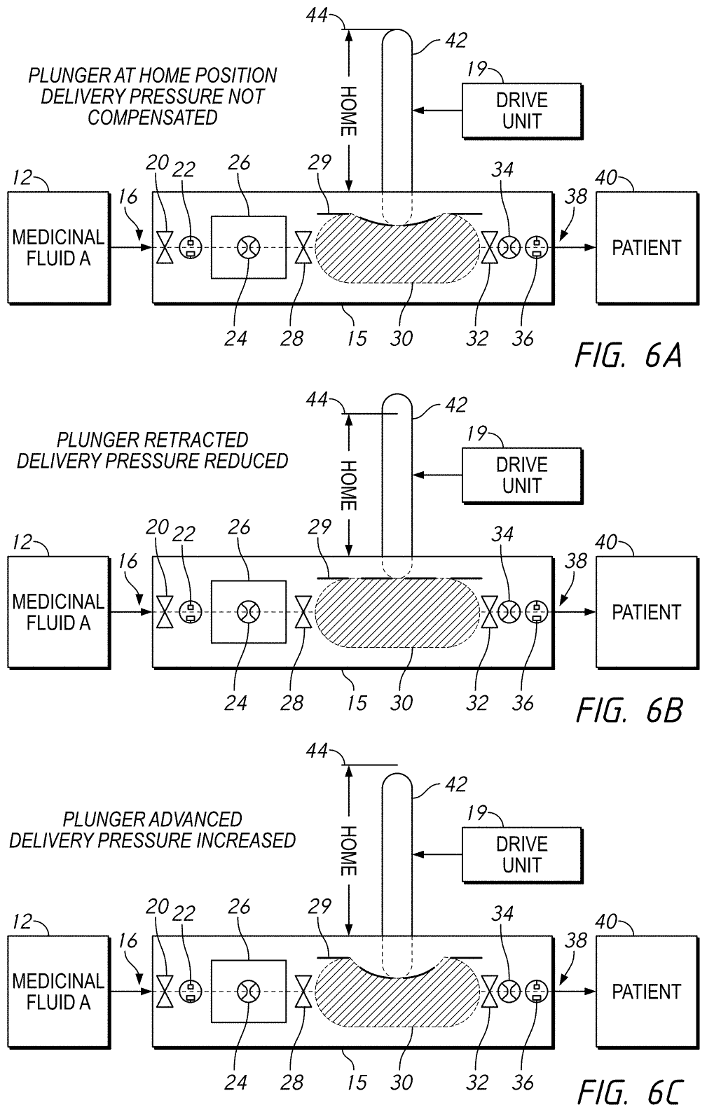

FIG. 6A-FIG. 6C illustrate how a change in the position of the plunger relative to the chamber affects the volume of chamber 30, and thus the pressure of the fluid within the chamber during a pump cycle. As explained in U.S. Pat. No. 6,497,680, a pump used to infuse a fluid can be controlled in accordance with an algorithm that enables a microprocessor to monitor and adjust each pump cycle to compensate for a differential pressure between the pump's inlet and outlet. The algorithm can define a fluid delivery protocol that is applied in controlling the operation of the pump to achieve a desired rate, volume, and timing of the fluid infusion. Fine pressure compensation adjustments, such as those described in U.S. Pat. No. 6,497,680 (and referred to here in the description of FIG. 6A-FIG. 6C), provide an example of specific operation details, unique characteristics, or features that can be encoded into a memory within a pump and used to improve accuracy of its predictions and displays to increase trust and accuracy.

The drive unit 19 of FIGS. 6A-6C can comprise a control unit and a stepper motor (see FIGS. 3C, 4C and 5B). For simplicity, only medicinal fluid A is shown in these figures. However it should be understood that alternatively, the described principles can be applied to compensate for a differential pressure of medicinal fluid B, or for a combination of medicinal fluid A and medicinal fluid B that may be passing through a multi-line cassette infusion pump.

In FIG. 6A, plunger 42 is shown in a home position (at the 0 step position). This position corresponds to the initiation of a pump cycle in which no differential pressure compensation is needed. Note that plunger 42 is in contact with the elastic membrane of pumping chamber 30, causing a slight deflection of the membrane. At the beginning of a pump cycle, plunger 42 is in an extend position at +169 Steps, outlet Valve 32 is closed, inlet valve 28 is open, and Supply valve 20 is in the open position (for Selection only of medicinal fluid A). Pumping chamber 30 is filled with the appropriate amount of medicinal fluid for the cassette pump, preferably about 333 μl for this embodiment, by retracting plunger 42.

When the algorithm determines that to properly compensate for a differential pressure, the delivery pressure must be reduced (i.e., because the proximal pressure is greater than the distal pressure), the plunger is retracted (while both inlet valve 28 and outlet valve 32 are closed) by the number of steps determined by the algorithm. Note that drive unit 19 preferably comprises a stepping motor (not separately shown), and it is therefore appropriate to refer to the displacement of plunger 42 in terms of steps of the stepping motor. FIG. 6B shows plunger 42 retracted to compensate for this differential pressure condition. Inlet valve 28 and outlet valve 32 are in their closed position, and it will be apparent that the volume of pumping chamber 30 has been increased (relative to its volume in FIG. 6A) due to the retraction of the plunger. Consequently, the pressure within pumping chamber 30 is effectively reduced before the plunger is displaced by the number of steps necessary to pump a nominal 333 μl of fluid.

Conversely, when the algorithm determines that the delivery pressure needs to be increased to compensate for the proximal pressure being lower than the distal pressure, the plunger is initially advanced into the chamber by an increment determined in accord with the algorithm. FIG. 6C shows that when the plunger is in this advanced position, pressure chamber 30 has a reduced volume. Therefore, the pressure of the medicinal fluid within pumping chamber 30 will be increased under these conditions.

### Multi-Line Pump Example

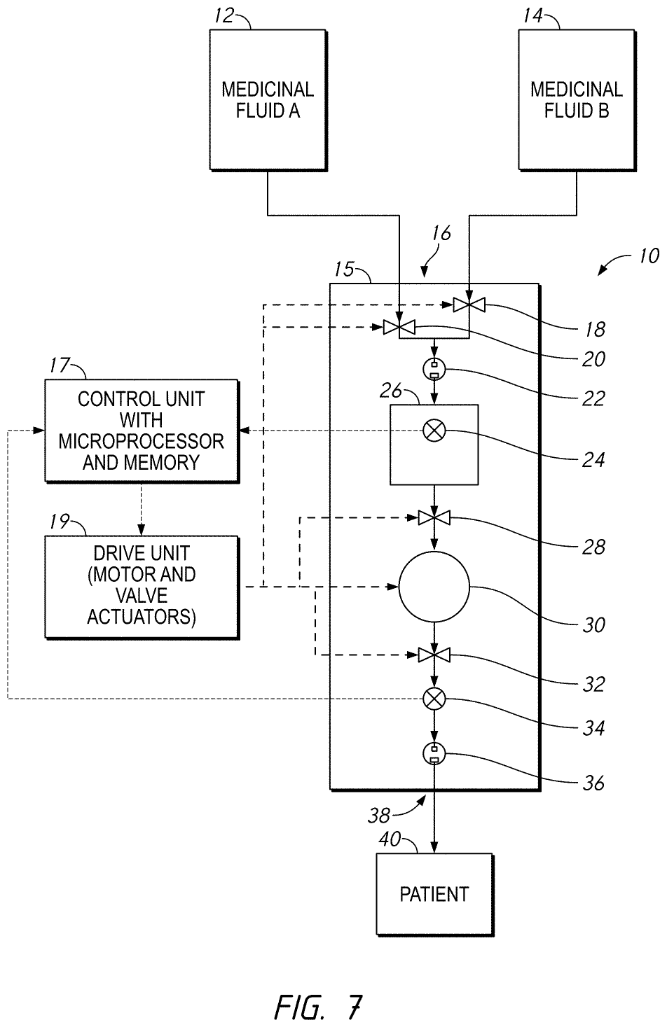

FIG. 7 is a schematic block diagram of a multi-line, pressure compensated cassette pump. Features shown here can correspond generally to those depicted in FIG. 3B. However, FIG. 7 also shows a control unit 17 and a drive unit 19, indicating how they interact with some of those features. In this figure, a multi-line cassette infusion pump 10 is shown. A source 12 of medicinal fluid A and a source 14 of medicinal fluid B are both coupled in fluid communication with a proximal end 16 of a cassette 15. The flow of medicinal fluid A into the cassette is selectively controlled by a supply valve 20, and the flow of medicinal fluid B is selectively controlled by a supply valve 18. If cassette 15 is to be used to pump only one of these two medicinal fluid at a time, only the appropriate supply valve 18 or 20 is opened to select the medicinal fluid to be pumped. The selected medicinal fluid (or fluids) then flow(s) through an air sensor 22 and into a mixing chamber 26. The purpose of the air sensor is to detect air bubbles that may be entrained in medicinal fluid A and/or B before the fluid is passed on into the pumping chamber and to the patient. Excess air bubbles entering a patient's bloodstream can cause an air embolism with potentially harmful consequences. A proximal (or inlet) pressure sensor 24 is disposed within mixing chamber 26. The selected medicinal fluid or fluids exit the mixing chamber through an inlet valve 28, when the inlet valve is in its open position, and into a pumping chamber 30. Details of suitable pressure sensors for use with the present disclosure and of other aspects of the cassette are disclosed in U.S. Pat. No. 5,554,115, the entire specification and drawings of which are hereby specifically incorporated herein by reference, for all that they contain for all purposes.

Cassette style infusion pumps are often constant displacement pumps. The volume of medicinal fluid in chamber 30 is therefore generally the same for each pump cycle. The differential pressure between the proximal and distal sides of the cassette can be compensated by increasing or decreasing the pressure of the constant volume of fluid within pumping chamber 30, as appropriate. As noted above, the preferable delivery volume of the medicinal fluid contained within chamber 30 is 333 μl—for this particular embodiment. Because of the small volume of the chamber, only a very small change in the relative volume of chamber 30 is required to provide an increase or decrease in the pressure of the medicinal fluid within the chamber. One side of chamber 30 is covered with an elastomeric membrane 29. Medicinal fluid is forced from pumping chamber 30 (when inlet valve 28 is closed and an outlet valve 32 is opened), by the action of a plunger 42 (e.g., as schematically shown in FIG. 5 and FIG. 6A-FIG. 6C) acting on the elastomeric membrane, forcing the elastomeric membrane into the chamber to displace the fluid contained therein. Adjusting the pressure within chamber 30 can easily be accomplished with an incremental change in the position of the plunger relative to the chamber before the start of a pumping cycle. In useful embodiments, the plunger position is variable from −489 steps to +220 steps, where a home position is defined to be at 0 Steps. A nominal stroke distance for plunger 42 to deliver 333 μl of fluid is +169 steps.

Inlet valve 28 and outlet valve 32 are formed in the cassette and are closed when rods or other actuating structures driven by drive unit 19 close off flow through the fluid passage of the cassette. When outlet valve 32 is in its open position, the medicinal fluid forced from the chamber flows through past a distal pressure sensor 34, through a distal air sensor 36, and exits the cassette to be conveyed to a patient 40. Multi-line infusion pump 10 also includes a control unit 17 and a drive unit 19. Control unit 17 preferably includes a microprocessor and a memory (not separately shown), however, it will be understood that the control unit can alternatively use other types of logic devices for implementing the algorithm, such a hardwired logic control, an application specific integrated circuit, etc. The algorithm is stored as a plurality of machine language instructions and data within the memory. The microprocessor receives information from distal pressure sensor 34 and proximal pressure Sensor 24, and implements the algorithm to determine whether the plunger position should be advanced or retracted to compensate for the differential pressure (see FIG. 6A-FIG. 6C). Drive unit 19 includes a prime mover (an electrical motor-not specifically shown) that is drivingly coupled to plunger 42, which forces fluid from chamber 30.

The algorithm compensates for a differential pressure detected between proximal end 16 and a distal end 38 of the cassette pump primarily by changing the position of the plunger relative to chamber 30 to increase or decrease the pressure within the chamber before the actual pumping stroke occurs. The algorithm can also change the timing of the pump cycle by controlling drive unit 19. Example details of a useful algorithm are discussed herein with respect to FIG. 6A-FIG. 6C.

The specific structure and operation details of a motor (e.g., the motor of FIG. 4) or other apparatus structure or operation (e.g., that of FIGS. 5, 6A-6C, and 7) provide examples of how a manufacturer typically has the most information about how its physical hardware works and how it will or will not respond to various situations. Any potential details or features of a given system are best known to a manufacturer, who can encode the resulting expectations into a memory within the pump itself. Indeed, a pump can be unique not only to a manufacturer, but unique to a particular batch, or even completely unique, when its performance is measured with sufficient accuracy and detail. Accordingly, a pump can carry with it, in its memory, information about its own response times, output volumes, pump mechanism displacement amounts, etc. It can take this information into account when displaying predictions, estimates, rates, arrival times, etc. A user (e.g., a clinician) can thus have enhanced trust in the accuracy of displayed or reported data.

### Pump Feedback Example

FIG. 8-FIG. 11 illustrate how a medical pump can have a closed loop stroke feedback system. FIG. 8 shows a cross-section view of a pumping membrane with integral, passive inlet and outlet valves (as opposed to those driven by a motor, actuator, and pin such as the valves 228 and 231 of FIG. 3C), but the sensor feedback principles from this example apply more generally. Medical pumps can include a means of determining and controlling their operating condition, including a means of adjusting a stroke frequency of a pump to compensate for individual variation in the stroke volume delivered by the medical pump. Such adjustments and compensations are described in U.S. Pat. No. 8,313,308 and provide examples of specific operation details, characteristics, or features that can be encoded into a memory within a pump and used to improve accuracy of its predictions and displays to increase trust.

The entire disclosure of U.S. Pat. No. 8,313,308 is incorporated by reference herein, for all purposes, for all that it contains. This patent describes a closed loop stroke feedback system and method for infusion pumps that can use pressure and positions sensors (see description in column 5 and FIGS. 2-4, for example). A pumping mechanism is illustrated in FIGS. 6-9, but as explained in column 1, pumps can include cassettes, syringe barrels, or tubing sections. This patent explains how adjusting stroke frequency can compensate for variation in stroke volumes. The outputs from the sensors and the results of the calculations described in the '308 patent (e.g., compliance of a pumping chamber) can provide device-specific information that is also used to further calculate and alert clinicians about what to expect for infusate arrival, equilibrium, or related clinical effects.

A fluid or pumping chamber can comprise, be included in, or be defined by a cassette, syringe barrel, or section of tubing. The pump includes a pumping element that intermittently pressurizes the pumping chamber during a pumping cycle.

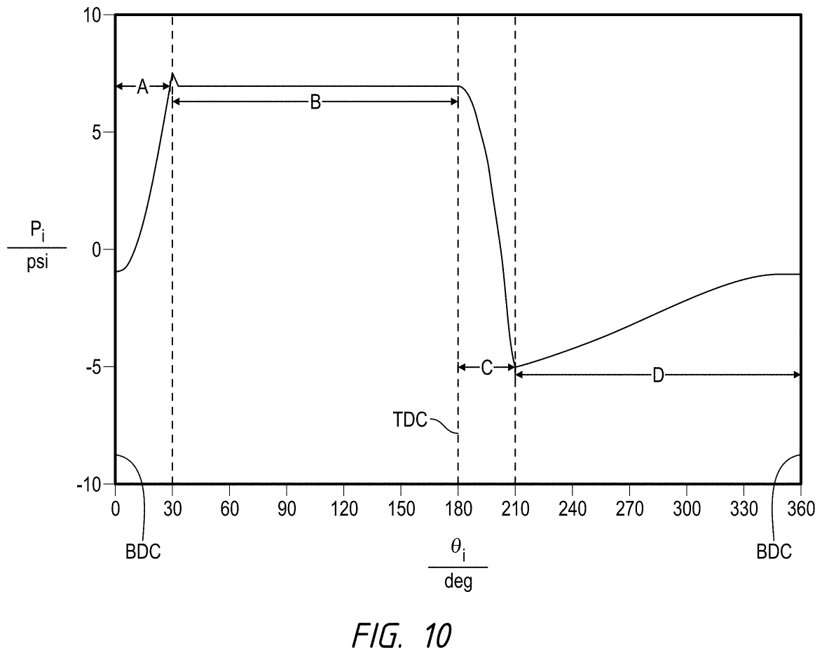

With reference to FIG. 8, a resilient elastomeric membrane or diaphragm 23 forms a pumping chamber 24, inlet valve 26, and outlet valve 28 on an inner face 68 of the main body 18. The pumping chamber 24 is connected in fluid flow communication between the inlet port 14 and the outlet port 16. The pumping chamber 24 operates to meter fluid through the cassette 12. Inlet valve 26 and outlet valve 28 react to the pressure of the pumping element 44 on the pumping chamber 24. FIG. 8 shows a sequence of four positions of a pumping element 44, with corresponding configurations of the resilient membrane 23 (and its contiguously-formed features such as the inlet valve 26 and the outlet valve 28). Both valves are closed in the top two configurations. The outlet valve 28 is open in the third configuration, and this is the pumping element 44's deepest penetration and displacement of the membrane 23 out of the four configurations shown. The inlet valve is open in the fourth (bottom) configuration. These configurations are also referred to in the description of FIG. 10.

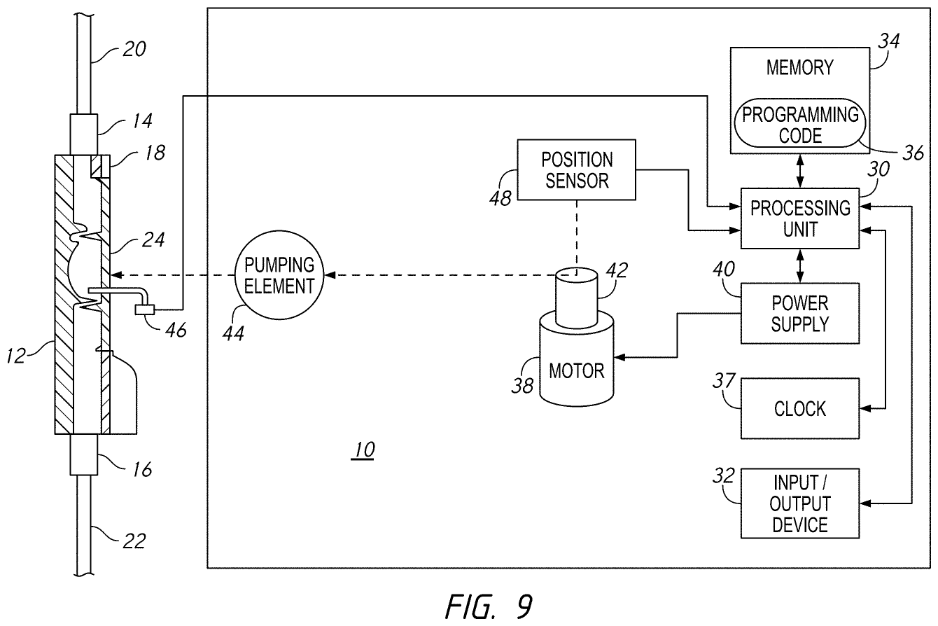

FIG. 9 schematically depicts a pumping system. A pressure sensor 46 detects the pressure exerted by the pumping element 44 on the pumping chamber 24. A position sensor 48 detects the position of the pumping element 44. A processing unit 30 processes pressure data from the pressure sensor 46 and position data from the position sensor 48 to determine a calculated stroke volume of the pump for a pump cycle, and to adjust a stroke frequency of the pump to compensate for variation in the stroke volume. In operation, the processing unit 30 sets a stroke frequency for a desired dosage rate based on a nominal stroke volume, determines when an outlet valve 28 (see FIG. 8) of the pumping chamber opens, determines a calculated pressurization volume from a beginning of the pump cycle to the point when the outlet valve 28 opens, determines a change in pressurization volume by subtracting the calculated pressurization volume from a nominal pressurization volume, determines a change in stroke volume by multiplying the change in pressurization volume by a ratio of pumping chamber expansion under pressure at the middle of the pumping cycle to pumping chamber expansion under pressure at the start of the pumping cycle, determines a calculated stroke volume based on the change in stroke volume, and adjusts the stroke frequency to compensate for variation between the calculated stroke volume and the nominal stroke volume.

FIG. 10 is a graph plotting force data versus the pump plunger position from a pump cycle, cross-sectional snapshots of which are illustrated in FIG. 8. Such force and position data can be obtained from the position sensor 48 and the pressure sensor 46, both shown in FIG. 9, for example. An example force curve is shown where the pumping element 44 applies force pi (shown in psi units) to the pumping chamber 24 while moving essentially a constant cyclic (sine-wave) motion through 360 degrees θᵢ (shown in units of degrees) of rotation per cycle. The pumping element 44 always has sufficient force available from the motor 38 so that its speed is essentially independent of the force pi applied to the pumping element 44, and the outlet flow from pumping chamber 24 is not restricted.

The curve starts at 0 degrees or Bottom Dead Center (BDC) with the pumping element 44 deflecting the diaphragm 23 of the pumping chamber 24 a minimal amount at this point. The position of the pumping element 44 at 0 degrees, and the resultant displacement of pumping chamber 24 can be seen in the top configuration of FIG. 8.

Cycle portion A shows the pressurization of the pumping chamber 24, which in this example occurs from 0 to 30 degrees. During the pressurization cycle portion A, the pumping element 44 moves down into the cassette 12 of FIG. 8 and FIG. 9. This is called the pressurization stroke because fluid is compressed in pumping chamber 24 of the cassette 12, building force within the pumping chamber 24, while the outlet valve 28 remains closed. The position of the pumping element 44 between 0 and 30 degrees, and the resultant shape of pumping chamber 24 can be seen in the second configuration of FIG. 8.

A delivery cycle portion B begins when the force within the pumping chamber 24 is sufficient to open the outlet valve 28. During the delivery cycle portion B, the pumping element 44 moves further into the cassette 12, forcing open the outlet valve 28 and expelling fluids from the pumping chamber 24. The delivery cycle portion B is shown in this example as occurring from 30 to 180 degrees. The position of the pumping element 44 between 30 and 180 degrees, and the resultant opening of the outlet valve 28 can be seen in the third configuration of FIG. 8. The delivery cycle portion B ends at Top Dead Center (TDC), or 180 degrees of rotation.

This is followed by a depressurization cycle portion C. The pumping chamber 24 depressurizes, occurring in this example from 180 to 210 degrees, as the pumping element 44 moves out of the cassette 12 (referred to as the up-stroke, depressurization or inlet stroke) and the force drops off. The resilient membrane 23 returns to its initial position, while the inlet valve 26 remains closed. The outward (upward) movement of the resilient membrane, combined with the inability of the outlet valve 28 to compensate by moving in toward the pumping chamber 24, causes negative pressure to build within the pumping chamber 24.

A refill cycle portion D begins when the negative pressure within the pumping chamber 24 is sufficient to the open the inlet valve 26 (see FIG. 8, final configuration). During the refill cycle portion D, the pumping element 44 moves away from, or up out of the cassette 12. As the resilient membrane 23 returns to a non-distorted shape, pressure within the pumping chamber 24 drops with respect to the upstream pressure (to the left of the inlet valve 26). This “negative” pressure within the pumping chamber 24 sufficient to open the inlet valve 26 and draw fluids past that valve into the pumping chamber 24. The refill cycle portion D occurs in this example from 210 to 360 degrees, or Bottom Dead Center (BDC). The position of the pumping element 44 between 210 to 360 degrees, and the resultant opening of the inlet valve 26 can be seen in the last (bottom) configuration shown in FIG. 8.

Information like that plotted in FIG. 10 can be used by a pump or other delivery system to predict when fluid will arrive at an infusing destination. Thus, a correspondence table calculating volumes, forces, effective rates, etc. can be stored in the pump or system's memory or generated in real time. This information can be incorporated into calculations (along with other inputs—for example, tube length between the pump and a patient) such that the pump or system can display predicted arrival times to a user, and other related information.

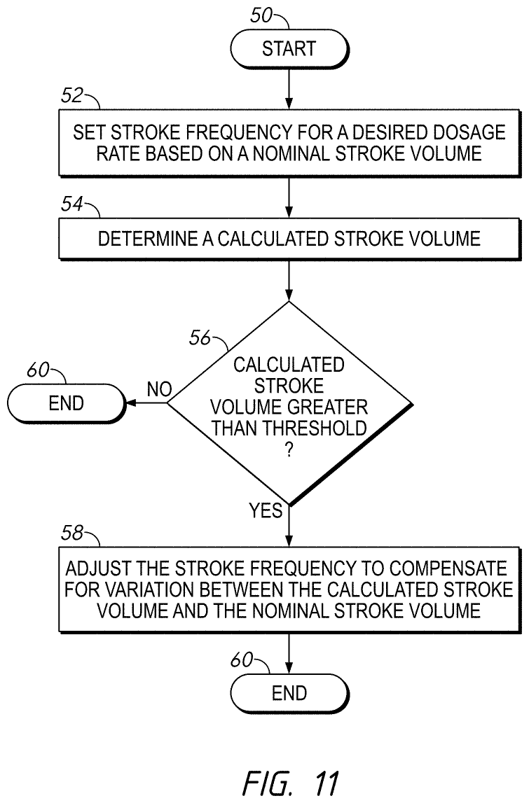

Referring to FIG. 11, a pump 10 can provide a mechanism for controlling or adjusting an actual delivery of fluid based on variations from nominal data used to estimate pump performance. The processing unit 30 retrieves the operating condition programming code 36 from memory 34 and applies it to the pressure and position data received during this pump cycle. The pump pressure data and pump position data are processed by the processing unit 30. Sensing the force that the resilient diaphragm 23 of the pumping chamber 24 exerts against the pumping element 44 and analyzing that force can determine an estimated volume of fluid flow per stroke (calculated stroke volume). The processing unit 30 may utilize the calculated stroke volume in a closed loop stroke feedback system. For example, it may modify the stroke frequency to compensate for variation in the stroke volume or make other adjustments. In some embodiments, stroke frequency timing is fixed for each flow rate; rates can also be changed by modifying stroke displacement, valve activity, etc. In the closed loop stroke feedback system, the processing unit 30 can adjust an actual delivery of fluid based on variation between the calculated stroke volume and nominal data used to estimate pump performance.

Specifically, the processing unit 30 begins execution of the programming code 36 at a block 50 and proceeds to block 52 where the processing unit 30 sets a stroke frequency for a desired dosage rate. The stroke frequency is determined by the processing unit 30 based on a nominal stroke volume. This nominal stroke volume can be supplied from empirical evidence of an average normal stroke volume for all pumps of a particular type or for each individual pump. Once the stroke frequency is set, the processing unit 30 proceeds to block 54 where it determines a calculated stroke volume of the pump for a pump cycle based on the pressure data from the pressure sensor 46 and position data from the position sensor 48. Once the calculated stroke volume has been determined, the processing unit 30 proceeds to decision block 56 where it determines if the calculated stroke volume is greater than a given threshold value. One of ordinary skill in the art will understand that the threshold value disclosed herein is predetermined from experimental data, and will vary from pump model to pump model. If the result from decision block 56 is negative, then the execution of the programming code 36 by the processing unit 30 is complete and ends in block 60. If the result from decision block 56 is positive, then the processing unit 30 proceeds to block 58 where it adjusts the stroke frequency to compensate for the variation between the calculated stroke volume and the nominal stroke Volume. Once the stroke frequency has been adjusted, the execution of the programming code 36 by the processing unit 30 is complete and ends in block 60.

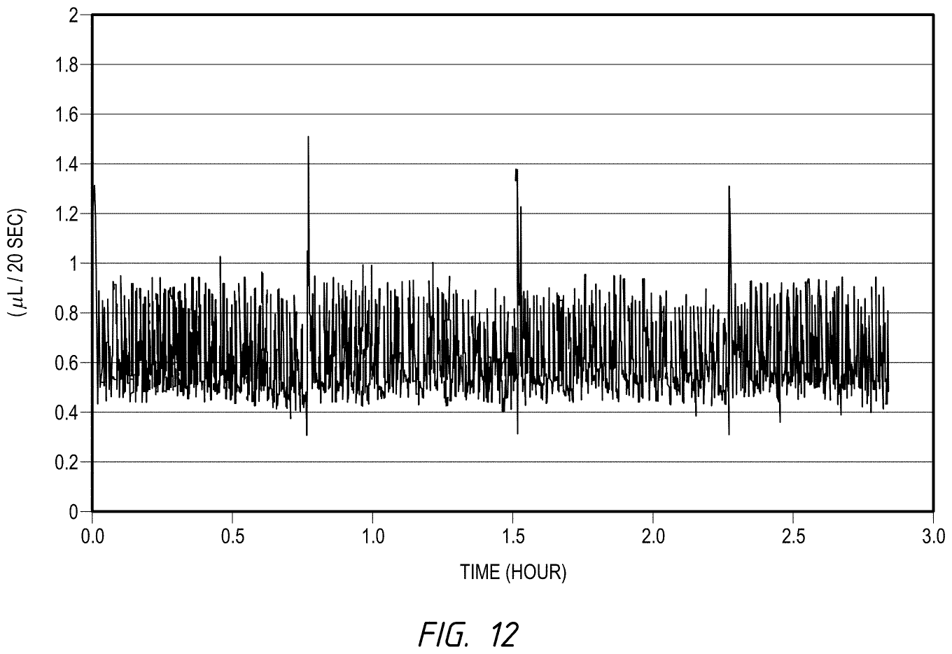

FIG. 12 of U.S. Pat. No. 8,313,308 illustrates a related approach, where the processing unit 30 adjusts an actual delivery of fluid based on variation between the calculated stroke volume and nominal data used to estimate pump performance.

In operation, closed loop stroke feedback systems can provide several advantages. First, the actual volume delivered per stroke can be used by the processing unit 30 to make adjustments (e.g., to continuously adjust the stroke rate or other parameters). Second, the detection of the pressure data profile and the determination of the opening of outlet valve 28 permits the processing unit 30 to determine lost stroke volume (i.e. calculated stroke volume as compared with the nominal stroke volume) and to use this as an indicator of presence of air in the pumping chamber 24, as well as deter mining the size of air bubbles in the set. These advantages of the present invention limit the effects of all causes of delivery error, including: compliance of physical components, air in the delivery fluid, variations in line pressure, and manufacturing variability of physical components (for example, in valve opening pressures).

In cassette type pumps, feedback is particularly advantageous. As the cassettes are disposable, the cassettes are often produced in very high volumes there are limitations to reducing the manufacturing variability of the physical components and assemblies. The overall accuracy provided by feedback and adjustment improves the ability to perform accurate deliveries within a broader range of these manufacturing variabilities of the physical components and assemblies. Feedback can help a pump automatically adjust its future operation or adjust displayed information to reflect a history or a future prediction.

A third advantage is that the detection of the pressure data profile and the determination of the opening of outlet valve 28 permits the processing unit 30 to deliver in smaller increments for very low flow rates in a more continuous manner (known as Low Flow Continuity). In general, Low Flow Continuity is defined as the ability of a pump to deliver at rates of 1 ml/hr to 0.1 ml/hr or less with periods of “no flow not exceeding 20 seconds and bolus volumes not exceeding 2 micro-liters.” To meet Emergency Care Research Institute (ECRI) industry standards for Low Flow Continuity and achieve an “Excellent” ECRI rating, a pump must deliver fluid in increments no greater than two micro-liters at a minimum pump-supported flow rate with a maximum “no-flow” period of 20 seconds.

The inputs or results from the feedback systems and protocols described here can be used by a pump or other delivery system to improve its displayed information and assist medical device users. Any adjustments to pump movement or control can be taken into account when reporting expected infusate arrival time, equilibrium, or a time for clinical effect to occur. This can be particularly important at low flow rates, because medical users may not understand the ultimate results or effects of pauses in infusion pump movement.

As shown in FIG. 12, a pump with a reciprocating plunger mechanism 44 can deliver fluid in small increments for very low flow rates in a continuous manner, which can comply with ECRI standards. FIG. 12 shows data for a pump delivering fluid with a low flow continuity of about 1 ml/hr or less, more specifically about 0.1 ml/hr or less, with twenty second incremental bolus volumes of less than 2 μl, using a feedback approach.

Whereas sensors and manufacturer information can have details of pump operation and structure, clinicians or other pump users do not always have immediate access to that information, or the time to interpret it for infusion or related decisions. Thus, a pump system can store that information and incorporate it into predictive or informational displays, thereby compensating for or preempting the risk of such potential user ignorance.

### Rate or Dose Catch-Up

The entire disclosure of U.S. Patent Publication No. 2015/0343141 (the '141 patent publication) is incorporated by reference herein, for all purposes, for all that it contains. This patent publication discloses how an infusion system can be configured for rate catch-up, and how this feature may depend on the type of medication infused.

Infusions can be described in two general categories or types: (1) intermittent infusions (not the same thing as purposely pulsed to achieve a low but “continuous” flow rate), which provide a set amount of medication for delivery into the body and are not generally rate-dependent, and (2) continuous infusions, which attempt to establish a direct patient response based on maintaining a consistent medication level in a patient using a particular delivery rate-such as a vasoactive that delivers dopamine to control blood pressure.

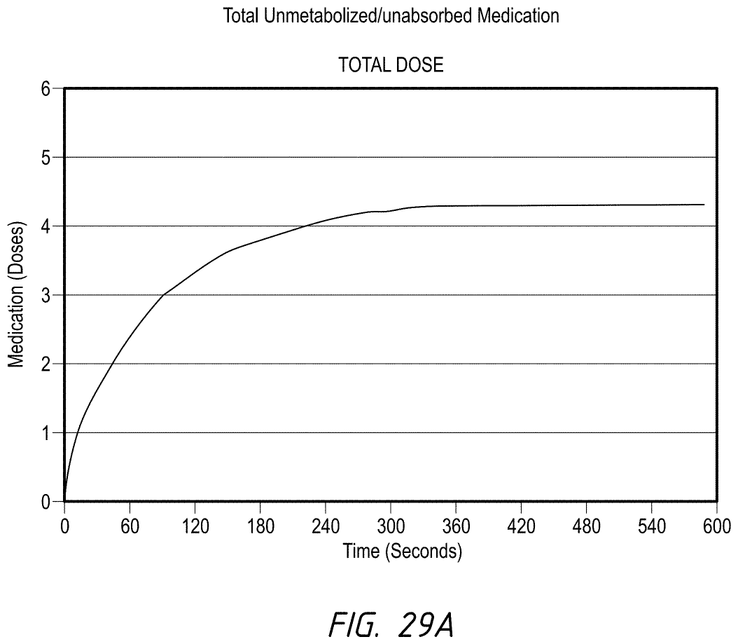

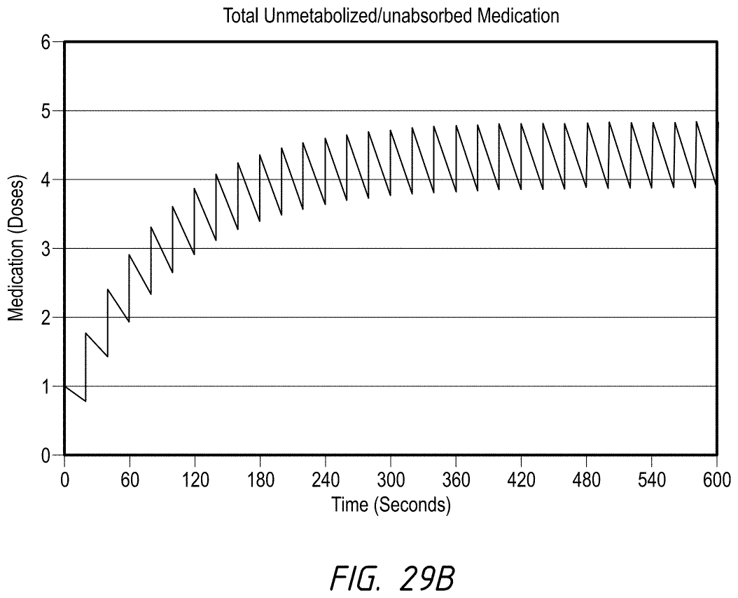

“Catch-up rates” in the '141 patent publication generally refer to compensating for lost volume delivered and are thus most relevant to completing a specified infusion volume within a specified time (intermittent delivery, the first category above). In contrast, catch-up “volumes” or “boluses” have more applicability for continuously delivered medications—the second category mentioned above. These catch-up volumes or boluses are not necessarily intended to compensate for a pause by catching up to an intended volume of delivery, but rather to use a dosage sequence that efficiently and safely re-establishes an effective or prescribed level of medication present in the patient on an ongoing basis—for example, to reestablish an equilibrium medication level. Further disclosure below is more concerned with the second category and the latter benefit: using catch-up volumes or boluses as a means of re-establishing an ongoing medication level in a patient. One of the benefits of the present disclosure is to track and provide insights to doctors regarding expected medication amounts in patients over time; while this does apply to intermittent medications, it is especially useful for continuously-delivered medications. FIGS. 29A and 29B both model “continuous” delivery (notwithstanding that FIG. 29B includes periodic pauses to achieve a lower overall rate and therefore may not seem smoothly continuous). A device can calculate and/or project in-patient amounts under both intermittent and periodic situations, and notify the device user. Infusion devices may use different modes of operation—e.g., intermittent or continuous-based on the type of medication infused. Intermittent mode may be used for a medication where a patient simply needs to receive a prescribed amount. Continuous mode is more typically used where the infusion rate of the medication (dopamine, for example) directly impacts a patient parameter (blood pressure).

Returning to the '141 patent publication, FIGS. 5A and 5B show example infusion pumps. FIG. 7 shows how rate catch-up can work by superimposing plots of a rate, actual infused volume, and target infused volume. FIG. 8 shows how pump structure can interact with a controller, sensors, and encoders in a feedback system. Page 5 explains that alarm values can be set and configured for catch-up rate factors, and for different drugs. The outputs from the sensors, the results of the calculations, and the settings and configurations selected, as described in the '141 publication, (e.g., catch-up rate factors, flags, and settings) can provide device-specific information that is also used to further calculate and alert clinicians about what to expect for infusate arrival, equilibrium, or related clinical effects. The disclosure relating to feedback and catch-up rates (primarily applicable to intermittent infusion, discussed above) can also be used to sense and provide feedback for catch-up boluses or volumes (primarily applicable to continuous infusion).

Infusion pumps are medical devices that deliver fluids, including nutrients and medications such as antibiotics, chemotherapy drugs, vasoactives, antidysrhythmics, inotropics, sedatives and analgesics, into a patient's body in controlled amounts. Many types of pumps, including large volume, patient-controlled analgesia (PCA), elastomeric, syringe, enteral, and insulin pumps, are used worldwide in healthcare facilities such as hospitals, and in the home. Clinicians and patients rely on pumps for safe and accurate administration of fluids and medications.

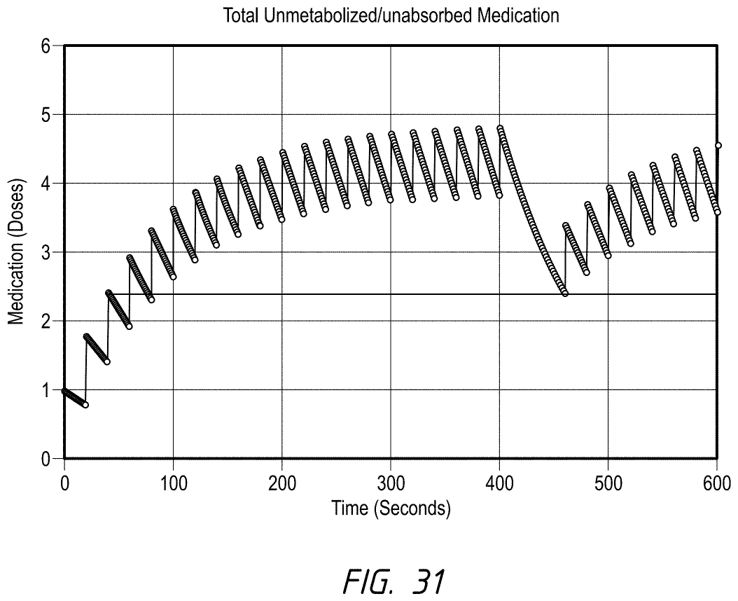

Infusion pumps can use an open loop pumping rate: the desired pumping or volumetric flow rate is input directly, or calculated from an input volume to be infused and delivery period or duration, and the infusion pump operates at a single target motor speed or stroke frequency to deliver the desired pumping or flow rate regardless of external conditions. Unfortunately, flow delivery can be interrupted by a variety of conditions, such as a stoppage or pause based upon a full or partial occlusion, a kinked tube, an air-in-line alarm, hanging a new IV bag, inserting new syringe with medication, vein clot, or the like. Once the flow delivery is interrupted, the time in which there is no medication delivery is lost, resulting in a delay in desired infusion completion. (FIGS. 31-34 provide example illustrations of this effect). Described are pumps and/or systems that can allow for catch-up rates or volumes. These can be specified by a user (e.g., as a simple percentage or range), but they can also be automatically calculated or optimized to account for the duration of any interruptions but also by comparison to whether those interruptions had an effect on the actual expected flow rate (when accounting for planned no-flow periods, for example).

Nurses typically work in shifts and may expect certain medications to be started and/or finished within their shift and plan accordingly. When occlusions, pauses, or other disturbances interrupt or delay medication delivery, this may disrupt the nurses planning for patient care within their respective shifts. In addition, the patients in these scenarios may not receive the medicine required within the allotted time. Infusion systems and pumps with configurable closed loop delivery rate catch-up can address these disadvantages. Moreover, a system that is aware of not only the disruptions, but the pump's response thereto (potentially triggered by one or more sensors) can provide more accurate predictions and other information through a pump display screen.

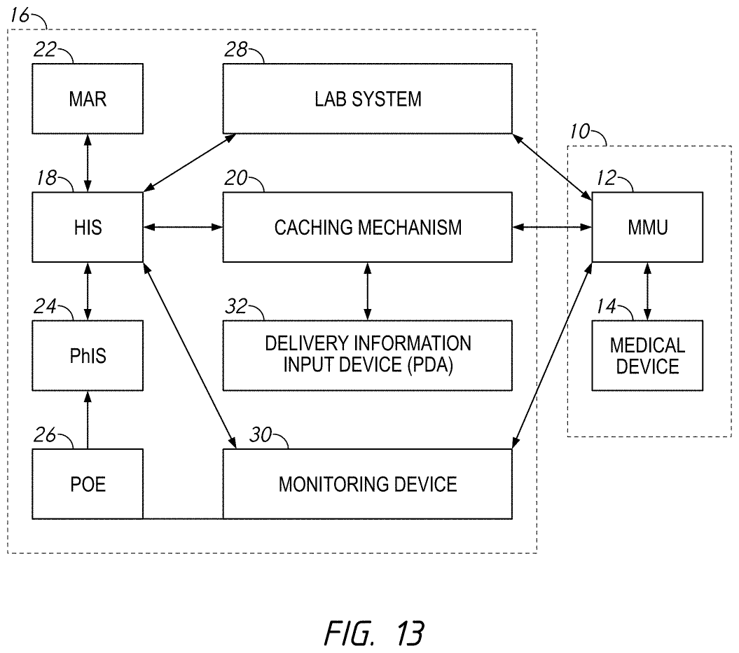

Returning to the present figures, FIG. 13 is a schematic diagram of a medication management system including a medication management unit and a medical device integrated with an information system. The medication management system (MMS) 10 includes a medication management unit (MMU) 12 and a medical device 14, typically operating in a hospital environment 16. The term hospital environment as defined herein is used broadly to mean any medical care facility, including but not limited to a hospital, treatment center, clinic, doctors office, day Surgery center, hospice, nursing home, and any of the above associated with a home care environment. There can be a variety of information systems in a hospital environment. As shown in FIG. 13, the MMU 12 communicates to a hospital information system (HIS) 18 via a caching mechanism 20 that is part of the hospital environment 16.

The caching mechanism 20 can primarily be a pass through device for facilitating communication with the HIS 18 and its functions can be eliminated or incorporated into the MMU 12 (FIG. 13) and/or the medical device 14 and/or the HIS 18 and/or other information systems or components within the hospital environment 16. The caching mechanism 20 provides temporary storage of hospital information data separate from the HIS 18, the medication administration record system (MAR) 22, pharmacy information system (PhIS) 24, physician order entry (POE) 26, and/or Lab System 28. The caching mechanism 20 provides information storage accessible to the medication management system 10 to support scenarios where direct access to data within the hospital environment 16 is not available or not desired. In one example, the caching mechanism 20 provides continued flow of information in and out of the MMU 12 in instances where the HIS 18 is down or the connectivity between the MMU 12 and the electronic network (not shown) is down.

The HIS 18 communicates with a medication administration record system (MAR) 22 for maintaining medication records and a pharmacy information system (PhIS) 24 for delivering drug orders to the HIS. A physician/provider order entry (POE) device 26 permits a healthcare provider to deliver a medication order prescribed for a patient to the hospital information system directly or indirectly via the PhIS 24. A medication order can be sent to the MMU 12 directly from the PhIS 24 or POE device 26. As used herein, the term medication order is defined as an order to administer something that has a physiological impact on a person or animal, including but not limited to liquid or gaseous fluids, drugs or medicines, liquid nutritional products and combinations thereof.

Lab system 28 and monitoring device 30 also communicate with the MMU 12 to deliver updated patient-specific information to the MMU 12. As shown, the MMU 12 communicates directly to the lab system 28 and monitoring device 30. However, those skilled in the art will appreciate that the MMU 12 can communicate with the lab system 28 and monitoring device 30 indirectly via the HIS 18, the caching mechanism 20, the medical device 14 or some other intermediary device or system.

Delivery information input device 32 also communicates with the MMU 12 to assist in processing drug orders for delivery through the MMU 12. The delivery information input device 32 can be any sort of data input means, including those adapted to read machine-readable indicia Such as bar code labels; for example a personal digital assistant (PDA) with a barcode scanner. Hereinafter, the delivery information input device 32 is referred to as input device 32. Alternatively, the machine-readable indicia can be in other known forms, such as radio frequency identification (RFID) tag, two-dimensional bar code, ID matrix, transmitted radio ID code, human biometric data such as fingerprints, etc. and the input device 32 adapted to “read” or recognize such indicia. The input device 32 is shown as a separate device from the medical device 14; alternatively, the input device 32 communicates directly with the medical device 14 or can be integrated wholly or in part with the medical device.

These devices and systems can work together to alert device users of expected outcomes. For example, an interface or display can be associated with the medical device 14 or the MMU 12, or both. One or more processors (e.g., in the MMU 12 and medical device 14) can receive input from an HIS 18, a lab system 28, a caching mechanism 20, etc. that is specific to a patient, a medication, a drug, and/or a medical condition. The processor can use such input to calculate expected drug metabolism rate, clinical effect, half-life, patient response time, equilibrium time, etc. These can be based on averages for other similar situations and/or models such as a multi-compartment (e.g., two-compartment) pharmacodynamic model. The system (e.g., MMU 12) can combine such calculations with measurements or other information about hardware processes (e.g., duration and/or number of pump motor or actuator pauses used to achieve a low flow rate, or history of pauses due to bubble alarms, disconnected or occluded lines, etc.).

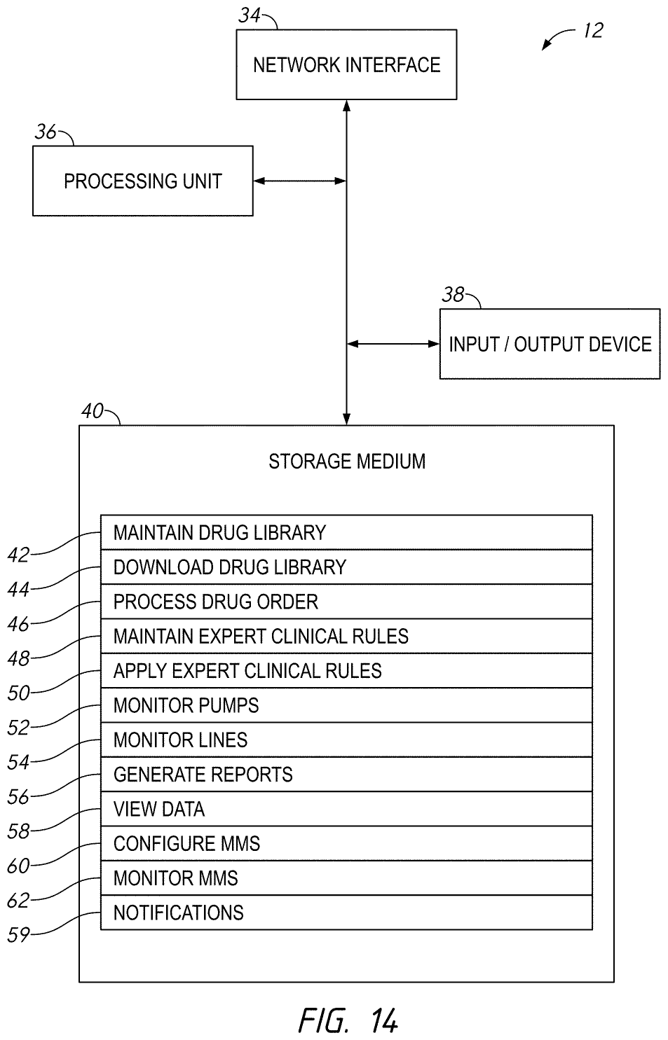

FIG. 14 is a schematic diagram of the medication management unit referred to in FIG. 13. The medication management unit 12 includes a network interface 34 for connecting the MMU 12 to multiple components of a hospital environment 16, one or more medical devices 14, and any other desired device or network. A processing unit 36 is included in MMU 12 and performs various operations described in greater detail below. A display/input or user interface device 38 communicates with the processing unit 36 and allows the user to receive output from processing unit 36 and/or input information into the processing unit 36. The display/input device 38 can be provided as a separate display device and a separate input device.

An electronic storage medium 40 communicates with the processing unit 36 and stores programming code and data for the processing unit 36 to perform the functions of the MMU 12. More specifically, the storage medium 40 stores multiple programs formed in accordance with the present invention for various functions of the MMU 12 including but not limited to the following programs: Maintain Drug Library 42; Download Drug Library 44; Process Drug Order 46; Maintain Expert Clinical Rules 48: Apply Expert Clinical Rules 50: Monitor Pumps 52: Monitor Lines 54: Generate Reports 56; View Data 58: Configure the MMS 60; and Monitor the MMS 62. The Maintain Drug Library 42 program creates, updates, and deletes drug entries and establishes a current active drug library. The Download Drug Library 44 program updates medical devices 14 with the current drug library. The Process Drug Order 46 program processes the medication order for a patient, verifying that the point of care (POC) medication and delivery parameters match those ordered. The Maintain Expert Clinical Rules 48 program creates, updates, and deletes the rules that describe the hospitals therapy and protocol regimens. The Apply Expert Clinical Rules 50 program performs logic processing to ensure safety and considers other infusions or medication orders, patient demographics, and current patient conditions. The Monitor Pumps 52 program acquires ongoing updates of status events, and alarms transmitted both near real-time and in batch mode, as well as tracking the location, current assignment, and Software versions such as the drug library version residing on medical device 14. The Monitor Lines 54 program acquires ongoing updates of status, events and alarms for each channel or line for a medical device 14 that supports multiple drug delivery channels or lines. The Generate Reports 56 program provides a mechanism that allows the user to generate various reports of the data held in the MMU storage medium 40. The View Data 58 program provides a mechanism that supports various display or view capabilities for users of the MMU 12. The Notifications 59 program provides a mechanism for scheduling and delivery of events to external systems and users. The Configure MMS 60 program provides a mechanism for system administrators to install and configure the MMS 10. The Monitor the MMS 62 program enables information technology operations staff capabilities to see the current status of MMS 10 components and processing, and other aspects of day-to-day operations such as system start up, shut down, backup and restore.

The storage medium 40 can include input useful for calculating expected drug metabolism rate, equilibrium time, clinical effect, half-life, patient response time, etc. The medium can also include sensor data or other information about hardware processes (e.g., duration and/or number of pump motor or actuator pauses used to achieve a low flow rate, or history of pauses due to bubble alarms, disconnected or occluded lines, etc.). The processing unit 36 can perform calculations that combine these two diverse inputs to provide a user with predictions of infusate arrival, equilibrium, clinical effect, etc.

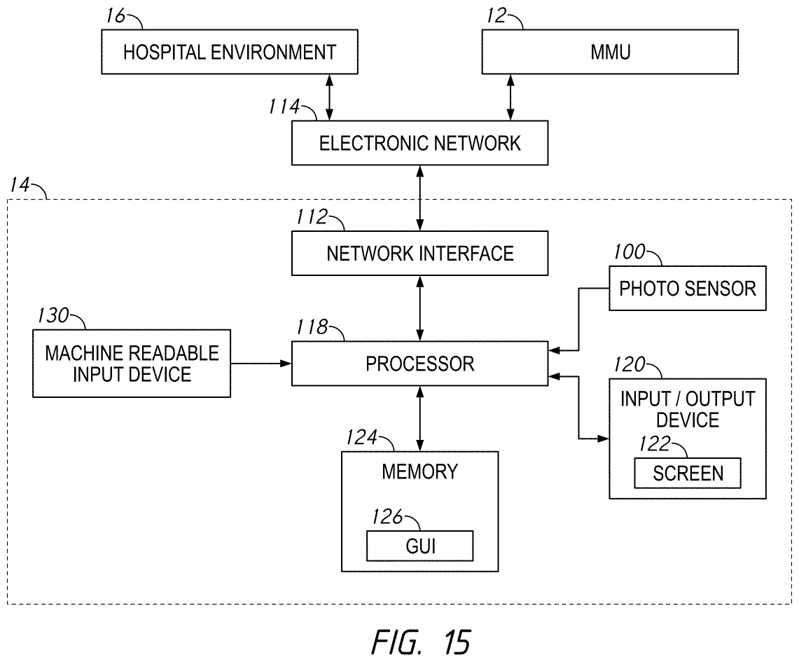

FIG. 15 is a schematic diagram of a medical device. An electronic network 114 connects the MMU 12, medical device 14, and hospital environment 16 for electronic communication. The electronic network 114 can be a completely wireless network, a completely hard-wired network, or some combination thereof. As used herein, the term “medical device” includes without limitation a device that acts upon a cassette, reservoir, vial, syringe, or tubing to convey medication or fluid to or from a patient (for example, an enteral pump, a parenteral infusion pump, a patient controlled analgesia (PCA) or pain management medication pump, or a suction pump), a monitor for monitoring patient vital signs or other parameters, or a diagnostic, testing or sampling device.

The pump style medical device 14 includes a network interface 112 for connecting the medical device 14 to electronic network 114. Where a wireless connection to the electronic network 114 is desired, network interface 112 operates an antenna for wireless connection to the electronic network 114. The antenna can project outside the medical device 14 or be enclosed within the housing of the device.

A processor 118 included in the medical device 14 can include a real time clock and perform various operations. The processor 118 can be especially useful in performing calculations and projections, and controlling information for display, in view of pump operating history information. It can update infusion display estimates or automate catch-up rates or doses or other behaviors, for example.

The input/output device 120 allows the user to receive output from the medical device 14 and/or input information into the medical device 14. Input/output device 120 can be provided as a single device such as a touch screen 122, or as a separate display device and a separate input device (not shown). In some embodiments, the display screen 122 of the medical device 14 is a thin film transistor active matrix color liquid crystal display with a multi-wire touchscreen. A membrane generally impermeable to fluids overlays the display screen 122 so the user can press images of keys or buttons on the underlying screen with wet gloves, dry gloves, or without gloves to trigger an input. The input/output device 120 can be especially useful displaying the results of calculations and projections, and controlling or reporting results from a processor 118, in view of pump operating history information. It can provide or allow adjustment of updated infusion estimates, catch-up rates or doses, or other behaviors, for example.

A memory 124 communicates with the processor 118 and stores code and data necessary for the processor 118 to perform the functions of the medical device 14. More specifically, the memory 124 stores multiple programs formed in accordance with the present invention for various functions of the medical device 14 including a graphical user interface program 126 with multiple subparts. The memory 124 can be especially useful in storing pump operating history information for use in updating infusion display estimates or automating catch-up rates or other behaviors, for example.

A machine-readable input device 130 communicates with the medical device 14 to input machine-readable information to the medical device 14. The machine-readable input device 130 can communicate, directly or indirectly, with the medical device 14 via a wireless or hard-wired connection. The machine-readable input device 130 can be a device that is separate from but associated or in communication with the medical device 14. The machine-readable input device 130 can be any sort of data input means, including those adapted to read machine-readable indicia, Such as a barcode scanner or handheld personal digital assistant (PDA). Alternatively, the machine-readable input device 130 can be operable to read in other known forms of machine-readable information, such as radio frequency identification tags (RFID), touch memory, digital photography, biometrics, etc.

### Sample Interface Features

FIG. 16 and FIG. 17 show example user interface elements for multi-channel medical devices (e.g., those that include more than one cartridge or cassette). In some embodiments, these can communicate with a machine-readable input device 130 (see FIG. 15). FIG. 16 illustrates a split screen display, having one portion associated with each channel. FIG. 17 illustrates an infusion pump with a screen display for receiving infusion programmable input from a user. The medical device 14 in this example is a multi-channel infusion pump. The medical device 14 can be a single channel infusion pump, a multi-channel infusion pump (as shown), a combination thereof, or the like, as desired for a particular application.

The medical device 14 is a multi-channel pump having a first channel 132 with first channel machine-readable label 134 and a second channel 136 with a second channel machine-readable label 138. A user of the medical device 14 can operate a machine-readable input device (see item 130 in FIG. 15) to select a channel from one or more channels 132 and 136, by scanning in the associated machine-readable label 134 or 138. The user selects the desired channel 132 or 136 by using the machine-readable input device 130 to scan a factory or hospital programmed, unique, machine-readable label 134 or 138 that is electronically generated and presented on the screen 122, preferably positioned near the respective channel 132 or 136. Alternatively, the machine-readable labels 134 and 138 are physically affixed to the medical device 14, preferably on or positioned near the channel 132 and 136, respectively. Since the machine-readable labels 134 and 138 are generated and/or can be stored in memory 124 by the medical device 14, the medical device 14 can associate the machine-readable labels 134 and 138 to the channels 132 or 136. The medical device 14 then allows the user to program and activate the selected channel 132 or 136. The user may also manually select the desired channel by touching an appropriate folder tab on the touchscreen. The folder tabs are labeled and/or physically arranged on the screen so as to be proximate to the corresponding channel 132 or 136.

In a further aspect of the wireless embodiment, the medical devices can periodically broadcast a unique wireless device/channel IP address and/or a self-generated unique machine-readable label (for example, a barcode) 134 or 138 that can also be presented on the screen 122. Alternatively, the machine-readable labels 134 and 138 are physically affixed to or posted on the medical device 14. Each medical device will correlate such broadcasted or posted device/channel IP addresses and/or barcodes with a particular patient, who is also identified by a unique machine-readable label (not shown) or patient IP address. The user associates the desired pump(s) or channel(s) 132, 136 with the patient by using the machine-readable input device 130 to scan the unique machine-readable labels 134, 138 and the patient's machine-readable label. This causes the appropriate pump processor(s) 118 to associate the appropriate pump channel(s) 132, 136 with the patient. Then the pumps or channels can associate, communicate, and coordinate with each other wirelessly.

The graphical user interface program reallocates screen 122 for the medical device 14. Specifically, FIG. 16 illustrates a multi-channel infusion medical device 14 with a split touch screen 122 having a first channel screen portion 140 associated with first channel 132 and a second channel screen portion 142 associated with the second channel 136. Each channel screen portion 140 and 142 presents a subset of the delivery information regarding the respective channels 132 or 136 including without limitation therapeutic agent name, concentration, dose rate, volume to be infused (“VTBI”), volume infused, and alarm information, in a font size that it is easily readable by a user from a distance such as, for example, from approximately fifteen to twenty feet (4.6-6.2 meters) away. This is what is defined herein as a “far view” delivery screen. The far view delivery screens display subsets of the information found on the relevant “near view” delivery screens. The near view delivery screen displays information such as, drug name, concentration, dose rate, time remaining, VTBI, volume delivered, and alarm name for the highest priority alarm if in an alarm state. A far view or near view screen can provide a user with a prediction for infusate arrival in a patient, equilibrium, and/or clinical effect or concentration at a destination, based on hardware-specific, rate-specific, drug-specific, operation-history-specific, patient-specific, or condition-specific, inputs (e.g., from a memory of a pump device or a related management device or system).

In practice, the delivery screen displays a near view when the user is programming the device as illustrated by FIG. 16. The near view delivery screen will switch to the far view delivery screen after a predetermined period of time that is predetermined by the manufacturer, configurable by the facility via the drug library, and/or set by the caregiver at the pump, for example after 20 seconds. Often, the user does not want to wait for the predetermined length of time to view the far screen.

Returning to FIG. 16, the channel screen portion 140 or 142 selected or corresponding to the tab selected expands in area but the size of at least some of the text therein is shrunk. The shrinkage of one of the channel screen portions 140 and 142 and enlargement of its counterpart provides additional space for one or more data display or data entry fields to be placed on screen 122, as shown in FIG. 17. Data displays or data entry fields are placed on screen 122 in space previously occupied by portions of the channel screen portion 140 or 142. This reallocation of space on screen 122 permits the user to enter inputs more easily since the data entry field can be large, preferably at least as large or, more preferably, larger in area than the original channel screen portions 140 and 142 were in the delivery screen mode. Additionally, the reallocation of space on screen 122 provides greater space for presenting information on the channel being adjusted or monitored.

Referring again to FIG. 16, the medical device 14 includes dedicated or fixed tactile infuser buttons, and images of buttons on the LCD-touch screen 122. The fixed tactile buttons 133, 135, 137, and 139 provide the following functions: LOAD/EJECT button 133—opens and closes the cassette carriage; ON/OFF button 135 turns power on and off; ALARMSILENCE button 137 silences an alarm for a specified period of time, for example two minutes; and EMERGENCY STOP button 139 stops all channels. The LCD color touchscreen 122 allows the user to access and use on-screen button images, for example 3D button images, and data entry fields. The touchscreen 122 uses a membrane over the LCD display so a single keypress does not cause significant infusion pole movement nor is it mistaken for a double keypress. The touch screen also accommodates a keypress whether the user is wearing wet gloves, dry gloves, or no gloves.

LCD touchscreen button images 143, 145, 147, and 149A-149E are located as shown in FIG. 16 and FIG. 17 perform the following functions: Patient Information Tab 143—dis plays the clinical care area, preselected patient information (including without limitation name, ID number, etc.), and provides access to a more detailed patient information screen; Channel Level Therapy Buttons 145—accessed by button images on the infuser touch screen, are used to select an infusion therapy; Program Level Buttons 147—accessed by pressing areas, drop-down list triangles, boxes or text boxes on the programming screen, are used to select dose parameters of an infusion; and Device Level Buttons 149A-149E at the bottom of the touchscreen are used to display and control device level features, including without limitation Mode 149A (for example, Operational or Biomed), Logs 149B, Locks 149C, Settings 149D, and Calculator display 149E. A wireless indicator image 102 displayed at the bottom of the screen 122 indicates that the medical device 14 is connected and ready for communication.

By using the Channel Level Therapy Buttons 145 and the Program Level Buttons 147, the healthcare practitioner can program each individual channel of the pump with specific fluid therapies in a variety of weight-based and body surface area-based units such as micrograms/kg/hour, grams/m²/hr, and other delivery specifications for the following modes: Basic Therapy—includes dose calculation, which allows dose rate programming based on Volume to be infused (VTBI), drug amount, infusion time and drug concentration and simple rate programming that allows programming of volumetric rate (mL/hr) based upon VTBI and time; Bolus delivery-allows user to program a single uninterrupted discrete delivery based on dose amount and time (the bolus can be delivered from the primary or a secondary container); Piggyback delivery-allows user to program the delivery of a secondary infusion, to be delivered through the same cassette as the primary infusion (the primary infusion is paused until the piggyback VTBI completes); and Advanced Programming. Advanced Programming mode can provide various types of programs, including Multistep-which allows a sequential delivery of fluid in up to 10 steps, with fluid volumes and delivery rates programmable for each step based on Rate and Volume or Volume and Time. Additional delivery modes can also be provided, such as: Variable Time-which allows up to 24 dose calculation steps at specified clock times; Intermittent-a calculated dose or step to be delivered at regular intervals; and Taper-a delivery that ramps up and/or ramps down to a plateau rate.

The memory 124 and the processor 118 of FIG. 15 can be used to track timing and effect of the tactile buttons and LCD touchscreen buttons discussed above, and this can be included in an operative history of a particular pump. The medical device 14 (or the MMU 12) can use such a history to adjust outputs that provide a prediction of infusate arrival, equilibrium, clinical effect, etc. (which can be displayed on the LCD-touch screen 122, for example).

Referring to FIG. 16 and FIG. 17, the graphical user interface provides channel indicators presented on screen 122. The channel indicators associate on-screen programming, delivery, and alarm information with a particular delivery channel by using graphical depictions such as a channel indication icon 154, 155. The channel indication icon 154 or 155 is a graphical item clearly associating on-screen programming, delivery, and alarm information with a delivery channel. The channel indication icons 154 and 155 are located on a tab 158 associated with a specified delivery channel of the medical device. The channel indication icon 154 or 155 may include but is not limited to a user readable letter or number, a machine-readable indicator 134, or a combination thereof. The graphical user interface also provides a drip indicator icon 160 and an infusion status icon 156 presented on screen 122.

Referring to FIG. 17, the screen 122 provides an optional drop-down box 170 for setting an Allow Rate Catch Up flag to one of an Enabled setting and a Disabled setting at the pump. The drop-down box 170 allows the user to enable or disable the rate catch-up function and to override the default rate catch-up flag provided in the drug library, if desired. When the Allow Rate Catch-Up flag is set to the Enabled setting, the user can enter a catch-up rate factor in the catch-up rate factor value box 172. In this example, the screen 122 also displays a catch-up rate factor limit value 174 and a catch-up rate factor alarm value 176 provided through the drug library. The Allow Rate Catch-Up flag can be replaced or supplemented with an “Automate Rate Catch-Up” setting. Such a setting can allow the pump or system to account for system-specific, patient-specific, drug-specific, or pump-history-specific factors, and avoid the potential for operator error (e.g., based on misunderstanding of pump operation at low rates). Such an automated setting can also be provided by default, without having a user-selectable flag. Similarly, an Allow Dose Catch-up functionality can be realized when appropriate, such as for continuous infusions.

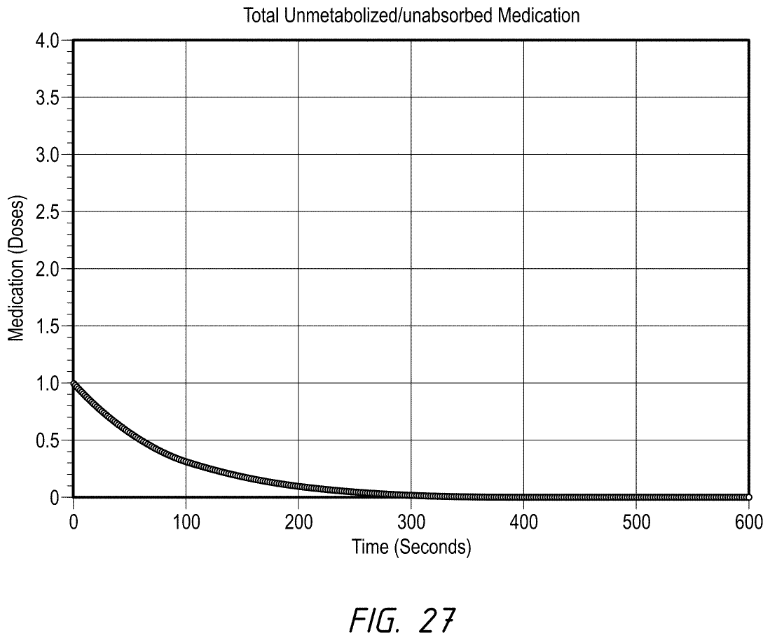

In this context, rate catch up can generally refer to compensating for lost volume delivered. For example, a catch-up rate can be useful to complete a specified infusion volume within a specified time, for what is often referred to as an “intermittent” delivery. However, similar icons, controls, and graphic user interface features can be used for catch-up doses, volumes, or boluses, in the context of “continuous” delivery. These catch-up doses, volumes or boluses are not necessarily intended to compensate for a pause by achieving an intended total delivered medication volume, but to use a dosage sequence that efficiently and safely re-establishes an effective or prescribed ongoing medication level in the patient. Various user interface devices can be used to indicate how close a medication is to achieving a desired steady state level, including: displayed percentages; colors (red is far from steady state, yellow is closer, green has achieved it); blinking (faster blinking is farther from steady state, slower blinking is closer); graphs (showing an expected level over time as it approaches a flat line showing desired steady state level); etc. FIGS. 27-34 provide examples of user interface graph features that can be used to indicate predicted or calculated medication levels to a clinician, for example. Complex detail as shown in FIGS. 27-34 may also be avoided by providing more basic or simplified insight to clinicians. For example, user interface indications (e.g., standardized icons, text, sounds, colors, etc.) to alert them to the following basic conditions: (1) an infusion has not yet reached the patient (for example when the downstream of distal line is primed with another liquid when the infusion of the medication of interest is initiated); (2) medication of interest is being delivered to the patient but has not yet reached a steady state level in the patient; (3) the infusion is expected to have reached a steady state in the patient; and/or (4) the infusion has deviated from steady state due to an unexpected flow discontinuity and is re-establishing equilibrium.

The catch-up rate factor limit value 174 can be the maximum catch-up rate factor which the user can enter in the catch-up rate factor value box 172, i.e., the maximum catch up rate factor or the soft or hard limit allowed for the particular therapeutic agent. The catch-up rate factor alarm value 176 can be a soft limit on the catch-up rate factor. In one example, the screen 122 will provide an alarm when the user enters a value in the catch-up rate factor value box 172 which exceeds the catch up rate factor alarm value 176, but the infusion pump will accept the catch-up rate factor after the user acknowledges the alarm or indicates a decision to override the Soft limit as long as the catch-up rate factor does not exceed the hard limit or catch-up rate factor limit value 174. Similarly, a catch-up dose factor and associated limits and alarms can be applied to a continuous infusion.

The catch-up rate factor limit value 174 and the catch-up rate factor alarm value 176 can be omitted or set to a high value as desired for a particular application. In some embodiments, the default values for the Allow Rate Catch Up flag setting, catch-up rate factor value box 172, catch-up rate factor limit value 174, and/or catch-up rate factor alarm value 176 can be loaded into the infusion pump from a remote computer as part of a drug library editing program such as ICU Medical MEDNET™ software. In some embodiments, the default values for the Allow Rate Catch-Up flag, catch-up rate factor value box 172, catch-up rate factor limit value 174, and/or catch-up rate factor alarm value 176 can be loaded into the infusion pump by the user at the infusion pump. In a hybrid embodiment, the default values can be established in a drug library downloaded to the pump and, if allowed by a setting in the drug library, later overridden or modified by the user at the pump. The entire catch-up rate behavior can be predetermined by the manufacturer and hard coded into the pump, without any user customization being allowed. Similarly, the catch-up dose factor, limit and default settings can be applied when appropriate, such as for continuous infusions.

The catch-up rate or dose factor (or allowed, proposed, default, or automated ranges or values for this factor) can be determined or influenced by data from a history of actual device operation or other device-specific parameters. For example, a device manufacturer may understand that when operated at a particular low rate, a pumping mechanism is inactive for intermittent, but relatively long, time periods. These long pump dormancy periods may by happenstance coincide with a user-forced or other incidental pause, such that no catch-up rate is warranted, despite a user's perception that a forced interruption disrupted the infusion flow. The device itself can analyze its own history to make this determination, avoiding unwarranted catch-up rates or doses or other adjustments. Moreover, the device itself can provide non-constant (e.g., tapering or smoothly changing) catch-up rates or doses that may achieve the desired levels or infusion rates more effectively than suddenly-changing catch-up rates or doses. These adjustments can be automatically determined, in view of a record stored in the device memory of its actual operation (and in view of the other relevant configurations, such as length and size of medical tubing between the pump and the infusate destination.

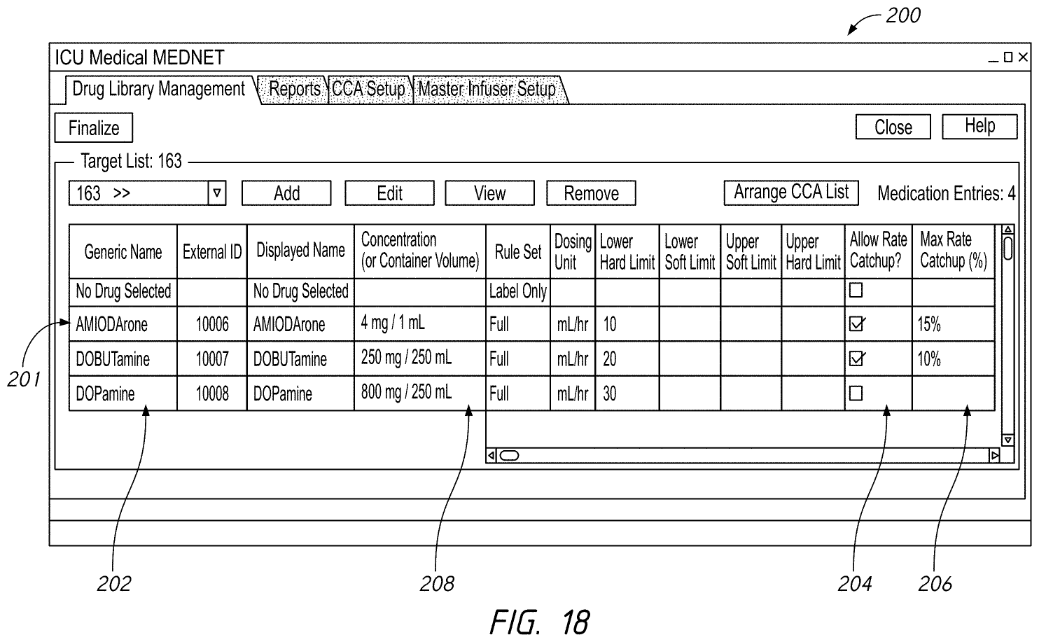

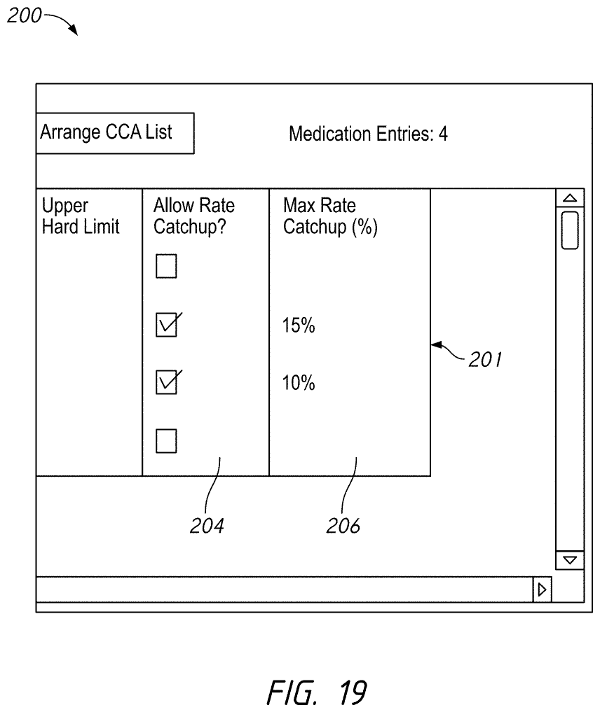

FIG. 18 and FIG. 19 show a graphical user interface and detail therefrom for configuring aspects of an infusion or pump system (e.g., for configuring a drug library). The graphical user interface 200 can be displayed on the display/input device 38 of the MMU 12 (see FIG. 14) and used to receive data for creating or updating the drug library, such as the catch-up rate factor, Allow Rate Catch-up flag setting, maximum catch-up rate factor setting, maximum catch-up rate factor alarm setting, and the like. A graphical user interface 200 can include a table 201 to receive different drugs and therapeutic agents in the drug library database. In this example, drug list 202 includes a list of the names of the drugs in the drug library, which could be the generic names, brand names or both. The drug list can include multiple entries for the same drug but different concentrations or clinical uses (cardiac, renal, pediatric). In this illustration, the Allow Rate Catch-Up Flag list 204 includes the allow rate catch-up flag setting for each drug/concentration/use entry (“drug entry” for short) in the drug list 202 to determine whether rate catch-up is allowed for the particular drug entry. However, in some embodiments, a catchup rate can be automatically allowed and also automatically determined or constrained. In such a case, the interface can provide information to a user about the rate, constraints, or calculation method. The Allow Dose Catch-up flag, limits and alarms could be implemented in a similar manner, when appropriate for a continuous infusion.

The Allow Rate or Dose Catch-up flag setting can be a function of pump type and/or clinical care area location. In this figure, the maximum rate catch-up list 206 includes the maximum catch-up rate factor setting for each drug entry in the drug list 202 for which rate catch-up is allowed. The maximum catch-up rate factor setting also can be a function of pump type and clinical care area location. In one embodiment, allowable maximum catch-up rate factors are regular linear percentages at predetermined intervals, e.g., 5%, 10%, 15%, 20%, et cetera. The table 201 can also include other parameters that constrain or limit the drug maximum catch up rate such as the normal global constraints on rate already configured via the MMU 12 and ICU Medical MEDNET™ software (Lower Hard Limit, Lower Soft Limit, Upper Soft Limit, and/or Upper Hard Limit). The maximum catch-up rate factor alarm setting and other drug maximum catch-up rate limits for each drug entry also can be a function of pump type, clinical care area location or specific medications. The Allow Dose Catch-up flag, factor, limits and alarms could be implemented in a nearly identical manner, when appropriate for a continuous infusion.

The catch-up rate or dose can be enhanced to minimize delay before a return to a desired equilibrium, in-patient concentration, flow rate, or clinical effect. This may require that no artificial or pre-determined maximum or similar constraints be imposed. An optimized catch-up rate or dose may be different, depending on how long an infusion pause lasted beyond the expected pause already inherent in a pump's operation at that rate. Thus, a catch-up rate or dose can be customized or determined on the fly—with reference to a pump's operation history that can be stored in the pump's own memory.

In FIG. 18 and FIG. 19, the catch-up rate factor is shown as a simple percentage, which can be applied to the selected basic infusion rate to obtain a catch-up infusion rate when the actual accumulated infusion Volume is less than the expected accumulated infusion volume. In some cases the desired infusion rate is input directly as a rate or volume per unit of time, such as mL/hr. In other cases the desired infusion rate is a calculated value based upon a dose and the weight or body surface area of the patient. For example, a dose of 10 mL/kg/hr can be prescribed for a patient who weighs 100 kg. Thus, the desired infusion rate would be calculated as 1000 mL/hr. In other cases the desired infusion rate is calculated based upon the dose and concentration of drug in the container. For example, if a dose of 10 mcg/hr is prescribed to be delivered from a 1000 mL container of fluid that has a concentration of 100 mcg of the drug, then the desired infusion rate is calculated at 100 mL/hr. There are other alternative dosing units for calculating desired infusion rates. In some embodiments, the catch-up rate factor is added to the desired infusion rate. In some embodiments, the catch-up rate factor (for example 1.05) is multiplied by the desired infusion rate. In some embodiments, allowable catch-up rate factors are regular linear percentages at predetermined intervals, e.g., 5%, 10%, 15%, 20%, etcetera to make it easier for the user to select a catch-up rate factor. The Allow Dose Catch-up flag, factor, limits and alarms could be implemented in a nearly identical manner, when appropriate for a continuous infusion.

In some embodiments, the catch-up rate factor can apply a simple linear adjustment to the desired infusion rate and does not rely on any input from physiological factors of the patient. Thus, the configurable closed loop delivery rate catch-up can be straight forward and avoid complex algorithms or control schemes. Instead the feedback mechanism of this algorithm can be based only on the measured versus expected accumulated volume delivered over time by the pump. The new rate Y is determined by a simple single order equation X-AX or AX; where A equals the catch-up rate factor as described above. The catch-up dose factor can be calculated based on the “lost” medication amount in the patient as a result of delivery pause and medication decay dynamics. Dose, volume or bolus “catch-up” delivery can be dictated by limits.

In some embodiments, however, the catch-up rate factor can be non-linear, dynamic, and/or determined in real-time with input from a system. These approaches can help reduce potential for user error or misunderstanding of how catch-up rates will effect clinical outcomes.

The Allow Rate or Dose Catch-up flag setting as a function of pump type can account for different pump types, makes and models, as well as uses for which various pump types are employed. In one example, one pump type using a cartridge and driving a plunger with a stepper motor can be used for general infusion such as saline solutions or the like, so that there is little risk in allowing rate or dose catch-up (especially user-selected, straight-percentage rate catch-up or modest, pump-calculated back-up doses). In another example, another pump type using a pre-filled syringe can be used for analgesics or opiates, so that it may not be desirable to allow rate or dose catch-up, unless the rate or dose catch-up includes some automation or safeguards such as those described herein. Other pump types may have multiple uses or therapies and it may be desirable to control enablement of (or to automate) the catch-up rate or dose feature for each of the plurality of uses available with such a pump type.

The Allow Rate or Dose Catch-up flag setting can be a function of clinical care area location. In one example, an infusion pump used in a treatment area in which the patients are in serious or critical condition, such as an emergency or operating room, may not want to allow rate or dose catch-up or may want to allow automated rate or dose catch-up. In another example, an infusion pump used in a treatment area in which the patients are in good condition may want to allow a more standardized or user-selectable rate or dose catch-up. Further, rate catch-up can be a function of the medication being delivered. For example, requirements for delivery of some medications such as antibiotics are not rate dependent per se, though there may be rate limits necessarily applied, but dependent upon a prescribed volume or dose being delivered to the patient. For these dose-based infusions, often referred to as intermittent medications, catch-up may not be necessary or desired unless useful to deliver the dose or volume in a prompt manner. However, some medications, such as vasoactives as an example, induce patient reactions that are directly related to the delivery rate; these medications are often referred to as continuous medications and pauses or temporary delays in deliver will benefit most from catch-up volume delivery to establish or maintain a level in the body. Automated catch-up boluses can be especially useful for low-flow pumps (e.g., those designed for use in newborn intensive care units with very small patients).

Automated catch-up rates or doses can take into account a pump operation history and patient-specific, condition-specific, or drug-specific parameters. The table 201 can include other data as desired for a particular application. The table 201 can include other exemplary columns 208 for additional data for the different drugs, such as External Drug ID Numbers, Drug Display Names, Drug Concentration/Container Volume, Selected Drug Rule Set (Label Only, Limited, Full), Drug Dosing Unit, Drug Dosing Limits (Lower Hard Limit, Lower Soft/Alarm Limit, Upper Soft/Alarm Limit, and/or Upper Hard Limit) or the like.

The drug library provides flexibility for various combinations of parameters as desired for a particular application. The drug library can have different Allow Rate or Dose Catchup flag settings for different drugs or drug entries in the drug library. The drug library can have different maximum permissible catch-up rate of dose factor settings for different drugs in the drug library. The drug library can have a given drug listed in multiple different clinical care areas (CCAs) in the drug library with at least one of different Allow Rate or Dose Catch-up flag settings and different maximum catch-up rate or dose factor settings for each given drug. A biochemical or physiological model can be used to estimate what will occur when a particular drug reaches its destination, and automated catch-up rates or doses can account for such an estimation or other calculations addressing biological or other clinical effects. These models will of course differ from drug to drug, so such information can be stored and/or displayed in association with entries supporting a drug library.

If a rate changes over time (either by initial program or by adjustment from a user or clinician), a catch-up rate or dose can be adjusted in a coordinated (e.g., proportional or other related) manner. Thus, a patient may need drug A in a higher dose for the first few hours after surgery and in a lower dose thereafter. The patient may need drug B in a lower prophylactic dose, subject to change if more pain is felt. For drug A, an automated or default catch-up rate or dose can proportionally track the overall dosage rate, going down in due course. For drug B, an automated catch-up rate or dose can similarly track any changes of a rate for drug B. Default catch-up rates or doses can thus be allowed to change to reflect the initial intent of a selected (or automatically set) catch-up rate, even if not manually changed to account for subsequent events.

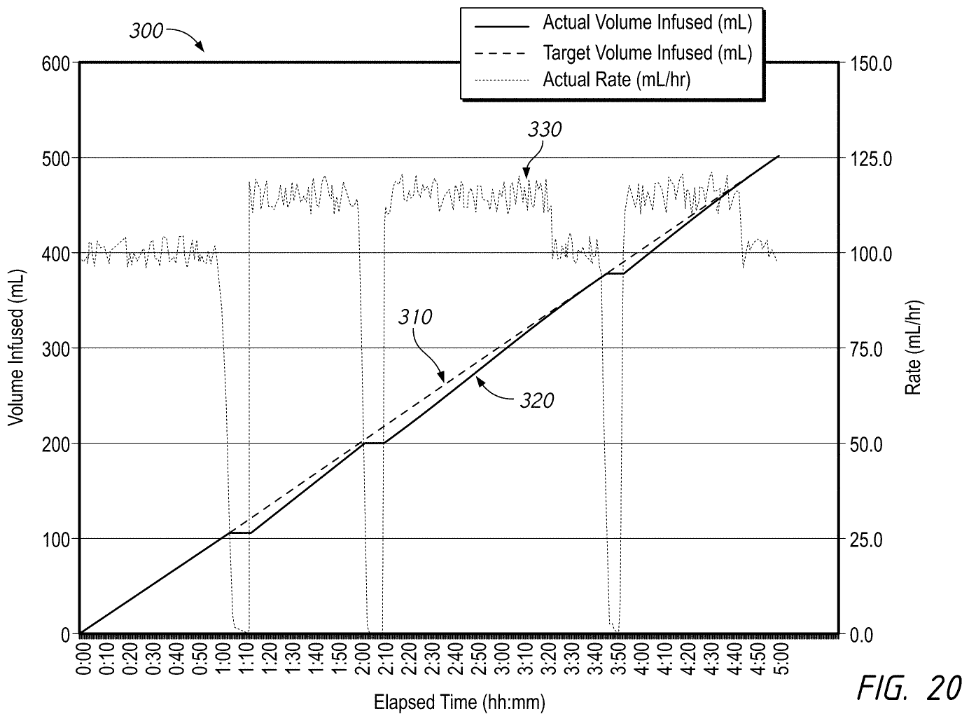

FIG. 20 is an example of a graph of an infusion volume and infusion rate versus time modeled for an infusion with an infusion pump employing configurable closed loop delivery rate catch-up. The graph 300 includes the expected accumulated infusion volume 310, the actual accumulated infusion volume 320, and the infusion rate 330.

The target accumulated infusion volume 310 increases linearly at a desired infusion rate of 100 mL per hour. The actual accumulated infusion volume 320 increases linearly from time 0:00 until time 1:00 at the originally programmed or desired infusion rate 330 of 100 mL per hour. At time 1:00, the infusion is interrupted so that the infusion rate 330 remains approximately zero and the actual accumulated infusion volume 320 remains about 100 mL until time 1:15, when the infusion resumes. At time 1:15, the actual accumulated infusion volume 320 is less than the expected accumulated infusion volume 310, so the infusion rate is increased by the catch-up rate factor of 15% and the infusion resumes at a catch-up infusion rate of 115 mL per hour from time 1:15 to time 2:00. At time 2:00, the actual accumulated infusion volume 320 has not quite yet caught up with the expected accumulated infusion volume 310 and the infusion is once again interrupted. The infusion rate 330 remains approximately zero and the actual accumulated infusion volume 320 remains about 200 mL until time 2:10, when the infusion resumes. From time 2:10 until time 3:20, the infusion is delivered at the catch-up infusion rate of 115 mL per hour until the actual accumulated infusion volume 320 equals the expected accumulated infusion volume 310 at time 3:20, when the infusion rate 330 is reduced to the originally programmed or desired infusion rate of 100 mL per hour. At time 3:45, the infusion is once again interrupted so that the infusion rate 330 remains approximately 0 and the actual accumulated infusion volume 320 remains at about 375 mL until the infusion resumes at time 3:55. From time 3:55 until time 4:40, the infusion is delivered at the catch-up infusion rate of 115 mL per hour until the actual accumulated infusion volume 320 equals the expected accumulated infusion volume 310 at time 4:40, when the infusion rate 330 is reduced to the originally programmed or desired infusion rate of 100 mL per hour. Thus, the desired accumulated volume of 500 mL has been delivered by the scheduled time 5:00 in spite of three interruptions in the infusion.

The illustration in FIG. 20 shows catch-up rates that are slightly higher than a standard rate (coinciding with times when the solid actual volume line 320 is separate from the dashed target volume line 310) and therefore ramp up until a target infused volume has been reached (the lines rejoin). However, a pump or other apparatus may not have data available to it showing when the target infused volume has been reached. Moreover, a target may be expressed in terms of a rate or an equilibrium level rather than a target volume. Accordingly, some catch-up approaches can involve tracking a pause duration, calculating its associated deferred medication volume (based on the rate), and after the pause, infusing the deferred amount of medication as a bolus. This may achieve a target volume most efficiently, but the bolus approach can cause an in-patient medication level to overshoot a target equilibrium level. Even if after a pause the deferred amount is metered back in at a lower rate than an immediate bolus, equilibrium may be reached without needing an entire deferred amount to be fully infused. Thus, “volume infused” may not be the most useful measure to achieve desired clinical outcomes (depending on the medication and other factors). Examples below show a catch-up bolus approach and various other problems, solutions and options for recovering after an infusion pause.

FIG. 21 is a block diagram of a control model for an infusion pump employing configurable closed loop delivery rate catch-up. This figure and the following description initially address a volume-goal approach for this control model. An alternative, equilibrium approach is discussed after that. The illustrated control model 400 includes an infusion volume calculator 410, a volume comparator 420, a pump controller 430, a pump drive 440, and a flow integrator 450. The infusion volume calculator 410 receives a desired infusion rate signal 412 and generates an expected accumulated infusion volume signal 414 from the originally programmed desired infusion rate signal 412 and the elapsed time. The volume comparator 420 receives the expected infusion volume signal 414 and an actual accumulated infusion volume signal 452, and generates a volume error signal 422 from the expected accumulated infusion volume signal 414 and the actual accumulated infusion volume signal 452. The pump controller 430 also receives the desired infusion rate signal 412 and the accumulated volume error signal 422, and generates a pump drive signal 432 from the desired infusion rate signal 412 and the accumulated volume error signal 422. The pump drive 440 receives the pump drive signal 432 to deliver the infusion 442. For modeling purposes, the pump drive 440 is subject to disturbances 444 which can cause or result in interrupted delivery of the infusion 442. The disturbance may include but are not limited to stoppages due to alarms, occlusions and other faults. The flow integrator 450 is operable to monitor the pump drive 440 and/or the infusion 442 and generate the actual accumulated infusion volume signal 452. In some embodiments, the pump drive 440 moves the plunger in a syringe and the flow integrator 450 senses pump drive/plunger position. In some embodiments, the pump drive 440 is a stepper motor and the flow integrator 450 counts pump strokes or motor steps. In some embodiments, the pump drive 440 is a rotary pump and the flow integrator 450 counts pump rotations. A drip counting device can also provide the necessary feedback concerning the actual flow rate and/or accumulated volume. Returning to discussion of the embodiment for driven pumps, the pump drive signal 432 is a function of the desired infusion rate signal 412 multiplied by a catch-up rate factor when the volume error signal 422 meets (equals and/or exceeds) a threshold that indicates that actual accumulated infusion volume is less than expected accumulated infusion volume, to catch up interrupted delivery of an infusion. The pump drive signal 432 is a function of the desired infusion rate signal 412 alone or returns to the originally programmed or set rate when the accumulated volume error signal 422 indicates that the actual accumulated infusion volume is greater than or equal to the expected accumulated infusion volume.

In some embodiments, volume diffused calculations and controls are adjusted to account for estimated metabolization, absorption, dispersion, or other results of expected biological processes. Thus, rather than (or in addition to) tracking and comparing running totals for expected versus actual volume infused (414 and 452 in FIG. 21), the control system can track and compare a volume within a certain past window of biological relevance, multiplied by a default rate of decay, to determine actual estimated infusate currently present at the destination (e.g., in a patient's blood stream), as compared to an expected amount for the current time at the destination. The default rate of decay can be provided with input from the results of multi-compartment pharmacodynamics modeling stored with drug information in a drug library onboard an infusion pump or controller memory, for example. The decay rate may also depend on patient-specific factors. The rate of decay may not be linear (e.g., it may depend on relative concentration, it may fall off more rapidly at first and slow down later, etc.). Because of such non-linearities and other effects, a comparison of total expected versus actual accumulated infusate volume may not yield the same result as a comparison of total expected versus actual infusate present at the destination. This disparity is increased if the expected volume infused 414 (depicted in FIG. 21 as resulting from elapsed time and initially programed rate) also fails to account for disturbances 444, which can come from the actual operation of the pump motor/plunger 440 itself.

Although one approach for obtaining feedback regarding infusate present at the destination is to provide invasive sensors there (e.g., in a patient) or to draw blood, these invasive approaches carry many additional risks and drawbacks. Accordingly, it is advantageous to provide predictions for infusate or medication levels (e.g., present or future) using the best information available in the pump itself for a particular pump, patient, medication, and past time window of pump operations. These predictions can be used to adjust pump operation (e.g., using catch-up rates as described with respect to the control model 400 of FIG. 21) or to provide information to a user (e.g., a clinician).

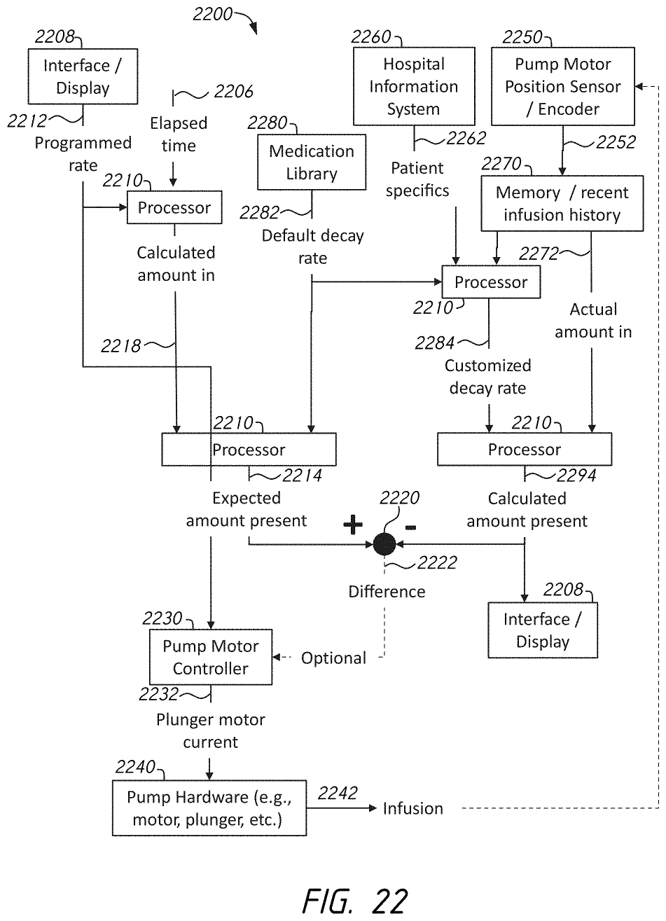

FIG. 22 shows a control and feedback system 2200 that can account for how a medication is used up through biological processes and for mechanical infusion pauses. An interface/display 2208 is shown for accepting a programmed rate 2212. This programmed rate 2212 and an elapsed time 2206 are fed into processor 2210. This results in calculated amount in 2218. A medication library 2280 can include a default decay rate 2282 (e.g., which can be an average rate for a given medication). This decay rate 2282 can be fed into a processor 2210 along with the calculated amount in 2218. (Hereafter “amount” is often used to refer to the medication amount present in the patient.) The processor 2210 can use these inputs to calculate an expected amount present 2214. This is different from a total volume infused (calculated amount in 2218, which generally corresponds to expected volume infused 414 in FIG. 21), because it has now accounted for what happens to a medication for example after it reaches a patient.

With further reference to FIG. 22, a hospital information system 2260 can include patient specifics 2262 which are fed into a processor 2210. These can include details like sensitivity to or metabolism rate for a particular medication, gender, weight, age, BMI, cardiac output, heart rate, breathing rate, blood oxygen saturation, core or external body temperature, SCVO2, etc. These can be relatively static or dynamic characteristics. They can come from a patient's long-term records, or from more dynamic or real-time monitoring of patient. This input need not come from a hospital information system but can come from the same or another medical device, for example.

The default decay rate 2282 can also be fed into the processor 2210 for purposes of determining a customized decay rate 2284. To do this, the processor 2210 can also take into account signals 2252 coming from a pump motor position sensor/encoder 2250. These signals 2252 can correspond to how much a pump has actually moved to urge fluid toward a destination (e.g., patient), and this information can be incorporated into a memory/recent infusion history 2270. This can be relevant for decay rate calculation because a medication's decay rate may be different based on a total amount or concentration of the medication in the blood stream, for example. The infusion history (especially the more recent portions thereof) can be used by the processor 2210 along with patient specifics 2262 and the default decay rate 2282 to achieve a customized decay rate 2284. For example, a patient may have recently received a large bolus or a high sustained infusion rate (which may give rise to a steeper expected decay rate), and this same patient may be prone to metabolizing the medication quickly or slowly due to a variation of their blood chemistry. The processor may be used to weigh these competing effects and calculate a customized decay rate 2284. This customized decay rate 2284 and the actual amount in 2272 during a recent, relevant period (available from the memory/recent infusion history 2270) can in turn be combined by processor 2210 to result in a calculated amount present 2294.

The pump motor position sensor/encoder 2250 and the memory/recent infusion history 2270 can take into account infusion pauses that may have resulted from a selected low programmed rate 2212 and/or from unplanned pauses (e.g., air alarm, kinked line or other occlusion alarm, pump repositioning, infusion line replacement, movement to another hospital room, replacement of syringe or IV bag, battery limitations, etc.). Thus, an actual amount in 2272 can be a trusted number. Also, the timing of delivery of increments of the actual amount can also be trusted.

The calculated amount present 2294 and the expected amount present 2214 are compared at 2220 and the difference 2222 can be optionally fed back in through a closed loop system into the pump motor controller 2230 which in turn can establish a plunger motor current 2232 that is used to cause the pump hardware 2240 (for example the motor plunger etc.) to in turn cause infusion 2242. This system can provide feedback for adjustments to a rate of infusion (e.g., through a catch-up rate after an infusion pause). This is an alternative to the closed loop hardware control system of FIG. 21, but keyed to track a potentially more clinically relevant parameter of expected amount of medication present in the patient rather than total infused volume. If desired, the results of the comparison can be displayed or recorded.

The illustrated control system and logic can also be used to simply display the calculated amount present 2294 on the interface/display 2208, alerting a user or clinician to a highly relevant data point for diagnosis, prognosis, care and treatment. A system such as described here does not necessarily require feedback from an in vivo sensor or other downstream source. Thus, a pump system can provide improved function (e.g., more accurate projections and outputs on an interface) by marshalling information already available outside a patient.

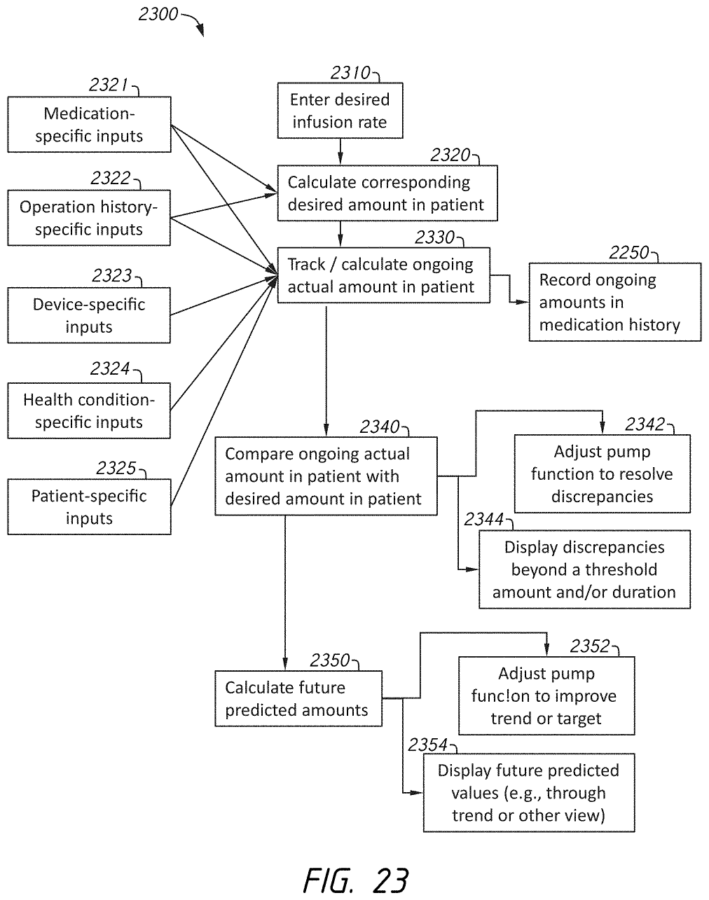

FIG. 23 shows how a system 2300 can operate to improve infusion and related display. At 2310 the system 2300 can allow a user to enter a desired infusion rate. At 2320 the system 2300 can calculate a corresponding desired or expected amount in a patient. This amount can correspond to a desired infusion rate using default inputs related to a rate of decay within a patient. At 2330 the system 2300 can track and/or calculate ongoing actual amounts in a patient. These amounts can be recorded in a medication history as indicated at 2250. At 2340 the system 2300 can compare ongoing actual amounts in a patient with desired amounts for that patient. As a result of this comparison, the system 2300 can adjust pump function to resolve discrepancies as shown at 2342. At 2344 the system 2300 can display discrepancies resulting from the comparison of 2340. In some embodiments, only discrepancies that exceed a threshold amount or and or a threshold duration are displayed. Displayed discrepancies can include, for example, the calculated amount in the patient, the calculated % of a delivered amount as a function of an eventual steady state level, and/or time remaining to achieve imminent steady state under the current infusion. Similarly, pump function may be adjusted only if discrepancies exceed a particular threshold. At 2350 the system 2300 can calculate future predicted amounts. These amounts can allow a system to adjust pump function to improve a trend or target as shown at 2352. At 2354 the calculation of future predicted amounts 2350 can result in a display of future predicted values, for example through a trend or other view.

Various inputs can be provided to the system 2300. For example at 2321 medication-specific inputs are shown and can contribute to the calculation of a corresponding desired amount in a patient 2320, and/or these medication-specific inputs 2321 can be input into a calculation 2330 of ongoing actual amount in a patient. These inputs can be stored and accessed from a drug or medication library (see, e.g., the discussion of medication library 2280 in FIG. 22 and similar disclosures elsewhere herein). These inputs can include information about drug half-life after infusion, for example.

Operation-history-specific inputs 2322 can be provided to the calculation 3220 of a corresponding desired amount in a patient, and/or they can be used to allow calculation 2330 of ongoing actual amount in a patient. Such inputs can include information about the duration and number of pump pauses that have occurred within a relevant past time window (e.g., the last 1 minute, 10 minutes, 30 minutes, etc.). These inputs can be especially relevant for determining the best information available, short of invasive sensors, for an actual medication amount in a patient because even if a clinician selected a desired rate 30 minutes ago, if the subsequent time period includes 10 minutes of bubble alarms and 10 minutes of an occluded tube, a standard 30-minute equilibrium level should not be expected. Similarly, these inputs 2322 can be useful for the desired amount calculation 2320 because if a previous time period included a very high rate but a clinician recently lowered the desired rate, the desired medication amount 2320 cannot be expected to fall into a much lower range immediately (e.g., within a minute of the dose reduction, for example). Thus, this input can keep the “desired amount in patient” 2320 within a reasonable expectation range and assist a user (e.g., clinician) to have appropriate patience, if needed. A medication history 2250 can provide feedback into operation-history-specific inputs 2322.

Device-specific inputs 2323 can be provided for the calculation 2330 of an ongoing amount. These inputs are not limited to calculations 2330 of actual amounts, since a device may have an upper flow rate limit that constrains what a clinician can select or enter at 2310. However, FIG. 23 shows that device-specific inputs 2323 can be especially useful for tracking ongoing amounts. For example, a device may know its own constraints (e.g., upper or lower infusion rate limits, actual flow characteristics due to inherent pauses at low rates, characteristics of a catch-up flow process such as when and if bolus amounts are infused, etc.).

Health-condition-specific inputs 2324 can, like medication-specific inputs 2321, be stored in and accessed from a medication library (see, e.g., the discussion of item 2280 in FIG. 22). For example, if a patient has particular diabetes type, an infusate (e.g., sugars or insulin) may be metabolized more quickly or slowly than would otherwise occur in healthy patients. Thus, these inputs can help determine a desired infusion rate 2310, or, more directly, can help calculate 2320 what a resulting desired or expected amount should be in a patient, given a selected infusion rate. (Once a pump rate is set, the system can calculate an expected steady state medication level in the patient; once this level is achieved, it can be referred to as an expected amount). For similar reasons, these inputs 2324 can also enable a calculation 2330 of an ongoing actual amount in a patient.

Patient-specific inputs 2325 can be stored and/or accessed from a hospital system (see discussion of patient specifics 2262 and the hospital information system 2260 in FIG. 22) and/or locally in a pumping device. For similar reasons to those discussed above, these can be provided to enable a calculation 2330 of an ongoing actual amount in a patient and/or to provide a realistic calculation 2320 of what an expected or desired in-vivo medication level would be for a given infusion rate. For example, a patient may have characteristics (male, obese, geriatric, infant, etc.) that affect not only expected medication concentration for a desired rate, but also the best information available on actual medication amounts in a patient (absent direct or in-vivo feedback of some kind).

Benefits of the above described systems include that various system inputs can greatly improve accuracy of predictions (and relevancy of any discrepancy calculations) without necessarily using in-patient sensors. Even if a pump device manufacturer does not make pump-specific details available for a software system as simple inputs, a system can be retro-fitted with a drip sensor or other apparatus (consistent with the feedback approaches described in U.S. Patent Publication No. 2015/0343141 discussed elsewhere herein) to determine the actual flow rate, the actual infused volume, the actual infusion history, etc. Output from such an in-line sensor can supplement or be substituted for the inputs 2322 of FIG. 23, the pump motor position sensor/encoder 450 of FIG. 21 and 2250 of FIG. 22, etc. Other benefits include the fact that measurement of medication levels or amounts in a patient may provide only a snapshot of this data, but without consideration of the pump activity in the appropriate time period before the measurement could mislead a clinician. For example, if pump pauses are not understood in this context, a clinician could utilize a single patient-measured data point and modify an infusion, unaware that the infusion may be appropriately re-establishing equilibrium or catching up for a previous pause or delay.

The described methods and systems can be performed using or incorporate an infusion pump having a processor and the memory coupled to the processor, the memory containing programming code executable by the processor to perform the steps of the methods illustrated in FIG. 21, FIG. 22, and/or FIG. 23, for example. In some embodiments, the infusion pump 14 (see, e.g., FIG. 16 and FIG. 17) can be in electronic communication with a medication management unit 12 and the catch-up rate or dose factor can be part of an updated drug library transmitted from the medication management unit 12 and received at the medical device 14.

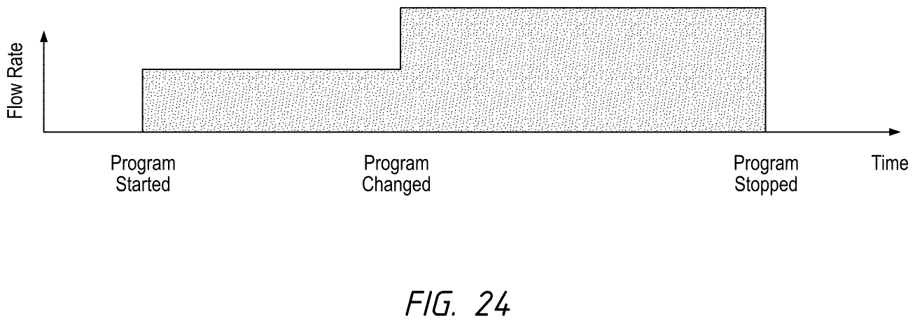

FIG. 24 shows how clinicians or other users may generally believe pumps operate. This graph plots flow rate on the vertical axis and time on the horizontal axis. A simplified view would consider that once a pump is programmed to deliver a substance, it will then immediately deliver the substance at the programmed rate as shown here. Thus, any titrations are immediately reflected in the delivery. When the program starts, an initial flow rate is used, and increasing the programmed flow rate immediately increases the actual flow rate, which continues until the program stops. Such a model may be relatively accurate at higher flow rates, but it does not reflect what happens at lower flow rates. To achieve low volume infusion, pumps typically include pauses in between deliveries of small boluses.

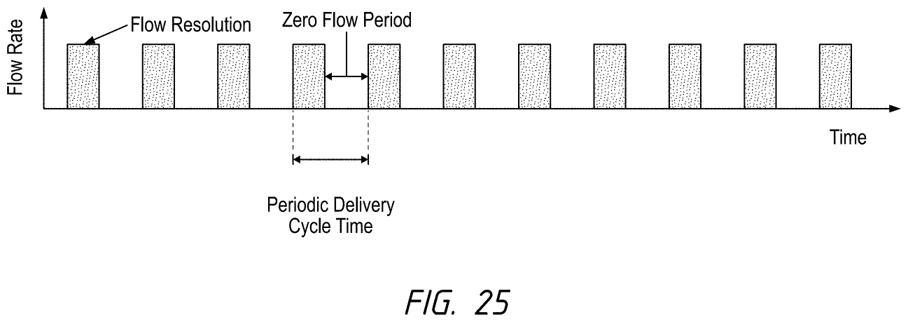

FIG. 25 shows how infusion pumps typically operate at low rates. Rather than continually infusing, at low rates small volumes or mini-boluses of fluid are delivered sequentially by pumps. Each time the pump motor turns, a small amount of medication is infused. This is the minimum flow resolution volume and is separated by “no flow” or “zero flow” periods. As shown, such low flow rates include periodic delivery cycle times, where a flow time and subsequent no-flow time is repeated. One reason for this approach is that mechanical pump apparatus may be difficult to control continuously at a low rate. For a pump that requires a turning mechanism—e.g., a peristaltic roller wheel, a syringe pump with a reciprocating piston mechanically associated with a displacement arm, etc. this can require a complicated gear box with significant ratios between initial and final gears, for example. Infusion pumps may infuse at between 0.1 and 1000 mL/hr or between 0.01 and 1000 mL/hr for syringe pumps, and a mechanical design to accommodate accurate delivery of this broad dynamic range with literally continuous delivery would be difficult. True continuous flow can thus be expensive to establish and maintain. Moreover, customized pump mechanics may be required for achieving true continuous flow at very low rates. By simply incorporating a periodic pause approach at a lower rate, standard pumping mechanics can be used for both higher and lower flow pumping systems.

The table below shows flow continuity (or, given the no-flow periods, flow discontinuity). Data was taken using various commercial pump examples.

| | Flow | | Programmed | | No Flow | | |
|---|---|---|---|---|---|---|---|
| Pump | Resolution | | Rate | | Period | | Notes |
| Example 1 | 2 | μL | 0.1 | mL/hr | 72 | seconds | 20 seconds or less no flow at 0.4 |
| | | | 1 | mL/hr | 7.2 | seconds | mL/hr or more flow rate |
| Example 2 | 2.12 | μL | 0.5 | mL/hr | 39 | seconds | Inconsistent flow profile < 20 |
| | | | | | | | sec. no flow at 1 mL/hr or more |
| Example 3 | 1 | μL* | 0.1 | mL/hr | 35 | seconds* | *Empirical measurement in early |
| | | | | | | | pump version; “no flow” |
| | | | | | | | period may now be longer |
| Example 4 | 2 μL, variable | | | | | | Doesn't scale no flow period |
| | | | | | | | linearly versus flow rate |
| Example 5 | 0.017 | μL (1 cc) | 0.01 | mL/hr | 6 | seconds | Theoretical flow resolution is |
| (Syringe | 0.108 | μL (5 cc) | 0.1 | mL/hr | 10 | seconds | dependent on syringe size and is |
| Pump) | 0.519 | μL (60 cc) | 0.1 | mL/hr | 50 | seconds | limited, variable due to stiction |
| Example 6 | 0.317 | μL | 0.1 | mL/hr | 11 | seconds | |
| Example 7 | 50 | μL | 0.1 | mL/hr | 30 | minutes | Meets IEC accuracy testing +/− 6%, |
| | | | | | | | though with only two fluid pulses per hour |

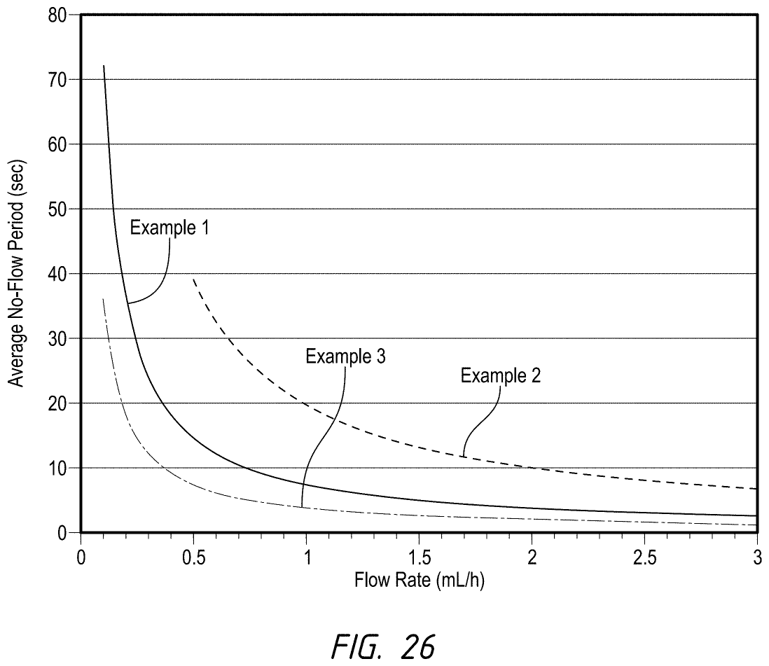

FIG. 26 shows three low flow continuity curves for three low volume infusion pumps. This figure plots average no-flow period in seconds on the vertical axis over flow rate in milliliters per hour on the horizontal axis for the first three example pumps from the table above. The table below shows example data for three selected flow rates for these same example commercial pumps.

| | Example 2 | Example 3 | | Example 1 | |
|---|---|---|---|---|---|
| Pump Resolution | 2.12. μL, variable | 1 | μL | 2 | μL |
| No-flow period @ 0.1 mL/h | N/A | 35 | sec | 72 | sec |
| No-flow period @ 0.5 mL/h | 39 sec | 7 | sec | 14 | sec |

| | | | | |
|---|---|---|---|---|
| No-flow period @ 1 mL/h | 20 sec | 3.5 | sec | 7 |

As shown, no-flow periods can be significant—up to 72 seconds in these examples and much longer in other pumps. No-flow periods last longer when rates are lower. The “no flow” curves in FIG. 26 are calculated from discrete resolution limit specifications. It is believed that subsequent versions of the apparatus used for Example 3 have substantially increased no-flow periods since this data was taken and these calculations performed.

Low-flow continuity (LFC) metrics such as those provided above, especially those with prolonged pauses, risk causing significant variation in clinical outcomes. For example, short half-life medications often have half-lives on the order of—or shorter than—these pump cycles at low rates, resulting in cyclical fluctuations of doses within the vascular system. Although not all medications have short half-lives in vivo, those that do are very often used in high-stakes situations in critical care. For example, many medications administered in neo-natal intensive care unit (NICU) and cardiac applications have short half-lives. Dopamine is an example, which can affect blood pressure. Additional transitory drugs (e.g., half-life of approximately 6 minutes or less when given intravenously) may include: Dobutamine, Dopamine, Epinephrine, Epoprostenol, Esmolol, Isoproterenol, Lidocaine, Nitroglycerin, Nitroprusside, Norepinephrine, Oxytocin, and Procainamide. The table below (see https://www.bettersafercare.vic.gov.au/resources/clinical-guidance/critical-care) has additional information for some of these examples.

| Medication | Half Life | | Reference |
|---|---|---|---|
| Dobutamine | 2 | minutes | https://www.safercare.vic.gov.au/clinical- |
| | | | guidance/critical/dobutamine |
| Dopamine | 2 | minutes | https://www.safercare.vic.gov.au/clinical- |
| | | | guidance/critical/dopamine |
| Epinephrine | 5 | minutes | https://www.safercare.vic.gov.au/clinical- |
| | | | guidance/critical/adrenaline-epinephrine |
| Isoprenaline | 2.5 to 5 | minutes | https://www.safercare.vic.gov.au/clinical- |
| | | | guidance/critical/isoprenaline |
| Nitroglycerine | 1 to 4 | minutes | https://reference.medscape.com/drug/glyceryl-trinitrate- |
| | | | iv-iv-nitroglycerin-nitroglycerin-iv-342278#10 |
| Norepinephrine | 3 | minutes | https://www.safercare.vic.gov.au/clinical- |
| | | | guidance/critical/noradrenaline-norepinephrine |

The risk of low flow discontinuity is being reviewed by various watchdog and third-party organizations. The nonprofit organization ECRI (founded as Emergency Care Research Institute) assigns an “Excellent” LFC rating instruments with pauses that last less than 20 seconds, and a “Good” rating for those with pauses that last less than 60 seconds. ECRI has affiliated with the Institute for Safe Medication Practices (ISMP) in connection with such patient safety ratings and studies. The Association for the Advancement of Medical Instrumentation (AAMI) is also reviewing this issue.

For short half-life and other transitory medications, establishing and maintaining dose equilibrium in patients can be very difficult (e.g., due to half-life issues, absorption and reaction rates). Device-specific titrations can also cause issues, as an average delivery rate changes over time but at low rates thru delivering periodic doses over modified timing intervals. LFC metrics matter because there is a linkage between accuracy and flow continuity in medication delivery that hasn't been considered fully in the past. The United States Food and Drug Administration (FDA) and AAMI may modify future pump performance evaluation criteria to reflect this.

To address these needs, pump systems can be improved. For example, system intelligence can be increased to recognize, analyze, and address the interaction between pump mechanics and medication and advise clinicians (and/or adjust further pump function) accordingly.

Infusing medication into a patient can have different goals. In some cases, a set amount of medication is to be delivered at a reasonable rate (intermittent infusion). In other cases, a medication is intended to be infused continuously to achieve an equilibrium level within the patient (continuous infusion). Whether the continuous infusion rate is high, medium, or low, a clinician may not be able to predict a medication level within the patient merely from the selected infusion rate. This inability results in part from complicated interactions between the fluid, pump hardware, pump control mechanisms, and tubing. It also results from complicated biological and other dynamic interactions between the fluid and the patient's body.

### Periodic Flow and Pharmacodynamics Modeling

FIG. 27 shows a plot of delivery of one pulse of medication, modeled as an impulse (dose is delivered immediately). In this model, half-life is 60 seconds and the “no flow” period is 20 seconds. The vertical axis is the amount of medication in units of doses, while the horizontal axis is time in seconds. The plot shows how the amount of total medication acting in a patient, which tends to decay over time (falling back to zero) unless it is offset by infusion of fresh medication. The plot does not show the total cumulative dose actually delivered.

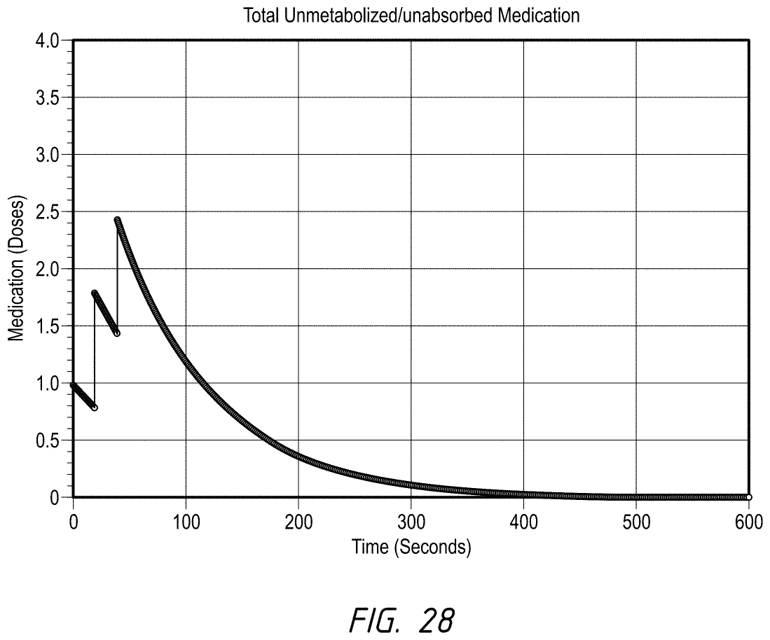

FIG. 28 shows uses a framework similar to that of FIG. 27, but this time with several pulses/doses of medication. Once again, half-life of the medication is sixty seconds, and “no flow” period is 20 seconds. (This is consistent for the subsequent examples as well). This plot shows that for a series of three doses, the total amount of medication acting in a patient reaches a higher level before decaying to zero.

FIG. 29A shows how continuous infusion can cause medication load in a patient's vessel to reach an equilibrium between 4 and 5 total doses. Ongoing medication delivery can offset decay in the medication level (e.g., through dispersion, absorption, metabolization, etc.). Rather than modeled as a series of doses (as with FIG. 29B), the medication amount depicted here smoothly rises to an equilibrium level. This model is representative of higher infusion rates where the discretization of infusion is not employed, and/or scenarios where system compliance is sufficient to “smooth out” any peaks and troughs in flow that may be the result of sequential delivery pulses. The plot shown in FIG. 29A can represent a higher infusion rate than that of FIG. 29B because the dose volume is not specified; doses of medication in the former may represent higher absolute volumes than doses in the latter, which achieves a higher overall infusion rate.

FIG. 29B shows how with pulsed infusion over time, in a series of doses, the total dose acting in a patient can also be increased to a generalized equilibrium level between 4 and 5 total doses. Here the persistent decay in medication level is more apparent as the plotted amount sinks after each dose in the ongoing series, but this ongoing decay is repeatedly offset by periodic dose delivery. Both FIGS. 29A and 29B depict a “continuous” infusion situation, which seeks to establish a steady state level of medication using a prescribed medication rate.

FIG. 30A shows red lines at two different times-approximately two minutes and approximately five minutes after medication infusion begins. A clinician may not be satisfied with a patient response during this time period (2-5 minutes), even though that response shouldn't yet be expected because the eventual equilibrium or steady state has not yet been reached. (A rule of thumb provides that it takes approximately five times a medication's half-life for equilibrium to be reached in a patient). In this example, there may be approximately plus or minus 10% variation in dose levels if no softening of pulsatile delivery is assumed. If a physician increases the rate prematurely, a patient may take in a greater dose (or reach a greater ongoing medication level) than is necessary or safe. That can in turn lead to a cycle of premature rate adjustments.

FIG. 30B illustrates a level of medication in a patient over time when a clinician changes an infusion rate multiple times before a steady state medication level could be expected to be achieved. In this scenario, a clinician may attempt to prematurely “chase” a patient response that cannot be achieved yet. This premature rate change by a clinician can be avoided using a system that provides better or more information to the clinician regarding the delivery of the medication over time, and the impact this provides on the amount of medication active in the patient. This figures depicts a continuous infusion example that is correctly programmed to deliver the desired steady state patient response, where the infusion set is initially primed with another liquid. Upon the start of an infusion, t=0, it will take time before the infusate of interest displaces the fluid in the line, after which the infusate of interest is actually reaching the patient. At 120 mL/hr, it would take approximately 5 minutes (300 seconds) from initiation of the infusion to displace a downstream volume of 10 mL, as shown at time A. (It should be noted that for syringe pumps even with a line completely primed with the infusate of interest, it can take long periods of time for fluid delivery to the patient to initiate due to compliance and mechanical slack in the system, which must be absorbed before delivery to the patient begins.) At time B, shown as just less than t=400 seconds, a clinician may recognize that the patient response is not adequate and therefore increase the delivery rate by 50%. Again, it is assumed that the half life of the infusate is 60 seconds, and the pump delivers pulses every 20 seconds. Although the initial rate would have over time, as shown in FIG. 29B or 30A, resulted in the appropriate steady state medication amount in the patient, the clinician has unnecessarily increased rate, which introduces a sharpened trajectory of increased total dose over time that overshoots the desired level. Furthering the example, at time C, shown as t=10 minutes (600 seconds) the clinician may recognize that the patient is over-responsive to the medication and reduce the rate by 50% (now at 75% of the original t=0 rate) and starting a decrease in the medication amount in the patient as the infused rate no longer compensates for the metabolization of previously infused medication, with the amount in the patient falling below the target amount level. Once more the clinician, “chasing” the patient response desired, increases the rate back to the original t=0 rate at time D=800 seconds and as of 15 minutes (900 seconds) the medication amount is approaching the target level. It has taken longer than necessary to “dial the patient in” and the patient has been subjected to over-delivery and under-delivery. This example represents one of many potential clinician pathways, many of which can result in longer overall time to achieve the desired patient response or more extreme over- and under-delivery. At any time, particularly at time A in this example, the ability for the pump to communicate to the clinician the calculated amount of medication in the patient, the relative percentage of that level versus its eventual steady state level if the infusion were not altered, or the calculated time remaining to achieve imminent steady state, could inform the clinician and improve the process.

FIG. 31 also shows a model with pulsed delivery over time in a series of doses. A sixty-second pause creates a drop down to a 50% dosage level, requiring several more doses (almost starting over) to re-achieve the higher equilibrium. This may be a result if an infusion pump detects air in the line (AIL) and initiates an AIL alarm protocol. For example, if an AIL alarm triggers, a pump may pause to allow a clinician to resolve the bubble. Even if the pause is merely for one minute, this may deprive a patient of the proper acting dose in their system for several additional minutes.

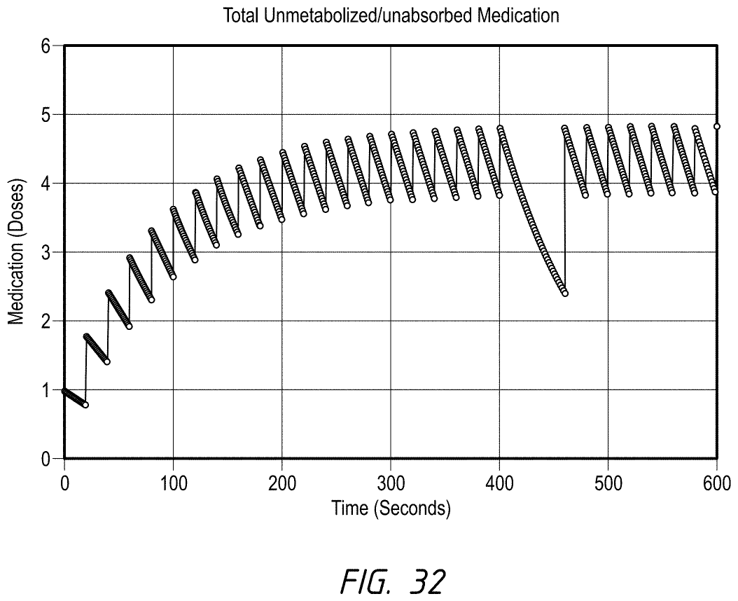

FIG. 32 shows how a pump may be able to automatically correct after a pause (e.g., 60-second pause) in pumping. For example, in a situation that begins similar to that described for FIG. 31, FIG. 32 shows how the pump can deliver a modest bolus (in this example, 2 pulses were missed and 2.4-times the typical fluid pulse was delivered in place of the third timed pulse). Because the pump system has information about the mechanics of its own pause and the decay dynamics of the medication, it is well positioned to provide a compensatory make-up dose that most efficiently re-achieves equilibrium.

Thus, in some embodiments, a method uses pump hardware input to resume an equilibrium in-vivo medication level. The method can include using a pump interface to receive a medication infusion rate and using the medication infusion rate and a stored medication half-life to automatically calculate a target in-vivo medication range having an upper bound and a lower bound. The method can periodically advance and pause a pump at standard intervals based on the received rate and the target in-vivo medication range. Pump hardware can be used to detect a non-standard infusion pause comprising a long interval that exceeds the length of a standard interval. In response to the detected pause, a pump system can measure the long interval and calculate a corresponding bolus amount of medication sufficient to achieve the upper bound for the target in-vivo medication range. Promptly after calculating, the pump can advance to infuse only the calculated bolus amount and then resume the periodic pump movement at standard intervals.

The principles described herein with respect to discrete steps (e.g., at low infusion rates where pump pauses can be easily measured) also apply to more smoothly continuous fluid delivery at a higher rate, where pulses are so frequent that they appear to provide continuous delivery (e.g., pauses are not present or discernible). Because medication levels in a patient are affected not only by pumping history and hardware control, but also by complicated biological and other dynamic interactions between the fluid and the patient's body, a system that accounts for both types of effects is useful for both high and low infusion rates. Another second reason the present disclosure is relevant to low and high flow systems and situations is that a clinician may not understand the threshold where a rate changes from low to high (e.g., stops having discernible pauses). A third reason is that a particular medication may be infused at both high and low rates at different times, or a system may be used for both high and low rate situations (for the same or different patients). Accordingly, a system that accounts for both system-specific characteristics and biological processes is highly valuable in practice, no matter what a particular infusion rate may be at a given time.

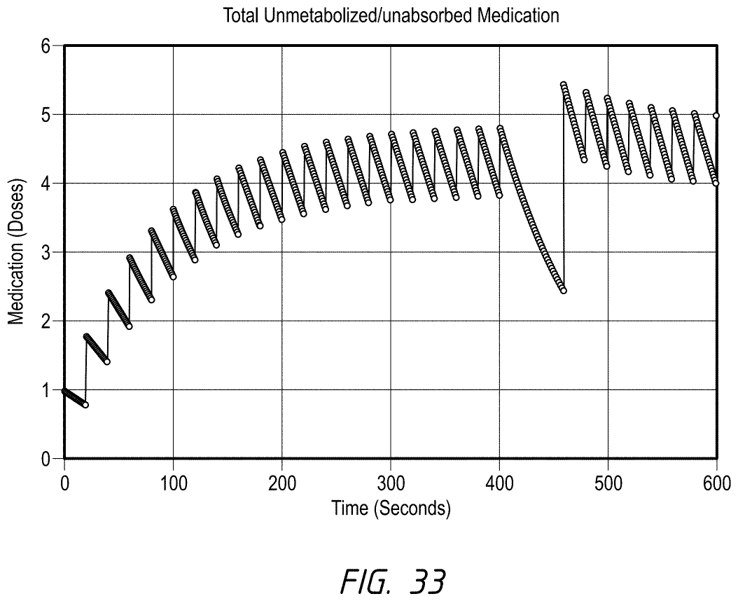

FIG. 33 shows another 60-second pause, followed by inclusion of all the missed medication in the next dose. This example assumes that two missed pulses are subsequently included in the next (3^{rd}) timed pulse. The result is that a patient overshoots the steady state level. A similar result occurs when a pump experiences a non-steady mechanical function (e.g., a syringe pump with stiction, or static then dynamic friction forces resulting in a jerky or sudden movement by a piston syringe).

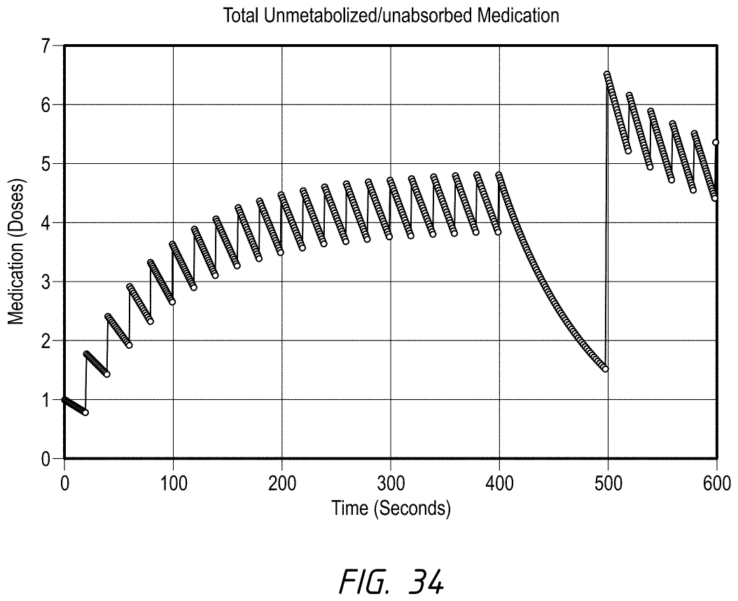

FIG. 34 illustrates an even longer (e.g., 100-second) pause. This shows that four missed pulses, all included in the next (5^{th}) timed pulse, causes an even more severe (40%) overshoot of the steady state dose level. The description included in the figures and text of this disclosure can thus be understood as recognizing several problems that occur with low volume pumps.

### Solutions

Improved systems can address these and other problems. For example, a pump system can understand and communicate to the clinician the expected state of the infusion in terms of dose or medication load in the patient. The pump can help instill rational patience in a clinician, based on a pump's internal operation information. A pump system can show that an infusion should not yet be expected to be in a dose equilibrium range (e.g., following initiation of the infusion, after a rate change, or after a standby/pause/alarm delay). Various graphical user interface or other means can be used to provide this background to a user. For example, a medication name can be highlighted or blinking on a screen, a dedicated on-screen icon can be provided, an alert window can be superimposed, stating that a medication was recently started/titrated if a titration was initiated, etc.

A pump can show the expected dose levels remaining after the pump has been placed into standby or stopped completely.

A pump can comprehend all delays inherent in its own apparatus and operating protocol. A pump can also store information regarding a larger system in which it participates and provide predictions of medication delivery that incorporate this information as well. For example, a pump may be able to gather information about the length or fill status of a fluid line between the pump and a patient in a hospital room. The pump's knowledge of its own mechanical pumping details, combined with knowledge about the length and volume of a relevant fluid passage, can allow it to predict and calculate delivery times with more reliability. The figures showing the models discussed above assumed various events would occur immediately, but additional time offsets are provided when a system is implemented for use. Delays a pump can “know” because they are typically inherent in the pump system include: time for the pump to reach target flow rate; time for medication to first reach the patient (downstream volume priming); and time to reach equilibrium dose level in the patient.

In addition to system-specific delays and parameters, a system can also calculate or address the time for patient to reach an expected physiological response in response to infusion. Onset of action and duration of action can also be important. As patients respond differently to medications, these types of delays are less predictable by models with machine-specific inputs and instead relay on pharmacodynamic models and principles of biochemistry.

A pump can track physical events that are not inherent in the system, but become known to the system over time. For example, a pump can report or compensate for pauses in delivery (e.g., resulting from alarms or clinician pause). In response, a pump can calculate, recommend and/or implement initial or catch up boluses or standby delays to reach desired in-patient dose levels. A pump can also or alternatively advise or strictly limit concurrent delivery of low rate medications (e.g., based on pharmacodynamics and the rates of both medications). For example, a database and memory can store pharmacodynamic data and be included in appropriate medical profiles in a stored drug library (DL), for example. Such data can be used to prioritize remote air or occlusion alarms for critical medications—for example, those with short half-lives infusing at low rates. A pump can advise or present lack of expected steady state. A pump can curtail (e.g., override, avoid, advise against, etc.) a post occlusion bolus, for example, which could introduce an overshoot of medication amount as shown in FIG. 33 and FIG. 34. Such smart features can be enabled or disabled for specific drugs through a custom drug library (CDL), for example.

Pumps can provide, report, or adjust for pharmacodynamic considerations of what is being infused. Although examples illustrated here may assume that the dose level of interest is based on simple half-life math, more complex “compartment models” may be used to predict dose levels over time for some medications. Moreover, some medications have half-lives dependent on the age of the patient, and some are impacted by degradation of liver or kidney function. In view of these nuances, some patient data can be integrated into a system that calculates, predicts, or automatically changes a dose or rate, for example.

Target controlled infusion (TCI) can allow clinicians to program target dose levels in patients, with the pump automatically delivering bolus/loading dose and subsequent maintenance or continuous infusion based on models stored in the pump to achieve the clinical objective. However, TCI may include risk when subsequent events cause earlier, model-based instructions to no longer apply.

Accordingly, an advantageous approach presents pharmacodynamic expectations of the infusion and includes recognition of when changes (e.g., mechanical issues such as stiction, kinds, alarms, user error, etc.) have introduced “missed” fluid pulses, which may have been followed by bolus-like delivery that increases dose beyond steady state. A system can use such expectations and recognition to establish customized, dynamic “bands”—or value ranges—for what defines an expected steady state range. Such bands can be configured based on the medication (e.g., lower half-life medications may have stricter or more calculation-intensive bands) or based on the clinical care area (CCA) (+/−10% of the average expected dose, +5%, /−20%). A system can also address scenarios for when a clinician will be alarmed (remote call back) versus provided with an alert (e.g., via on-pump messaging). Remote call back may be implemented, suggested, or allowed for critical, low half-life drugs, for example.

Disclosed embodiments can capitalize on the information available to a pump regarding its own mechanical structures and operating processes. Only the pump knows exactly how it is operating.

A pump system can also incorporate physiological feedback from a user (e.g., patient or clinician), for example. A pump can suggest and/or implement modification of infusion rates (dobutamine, for example) based on physiological response (e.g., blood pressure). This can be done automatically or using pump- or system-advised manual intervention. Alternatively or additionally, this modification can occur through closed loop control with physiological monitoring.

### Syringe Pump Considerations

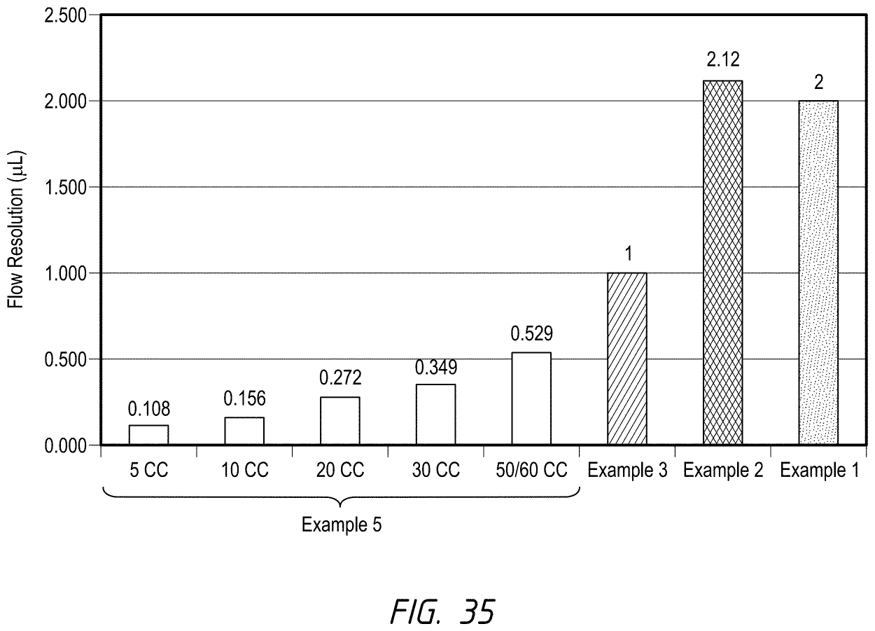

FIG. 35 shows how large volume (bag-based peristaltic or cassette-based) pumps generally exhibit larger flow resolution than syringe pumps, and therefore they have longer theoretical “no flow” periods than syringe pumps. Working from the left, the first five bars show flow resolution in microliters for five plastic syringes of increasing volumes using the same commercial pump as in Example 5 above.

| | Example 2 | Example 3 | Example 1 | Example 5 |
|---|---|---|---|---|
| Pump resolution | 2.12 μL+ | 1 μL | 2 μL | 0.108-0.529 μL |

The next bar plots a new Example 3 commercial device, with a 1 μL pump resolution, and the final two bars plot pump resolution for Example 2 and Example 1.

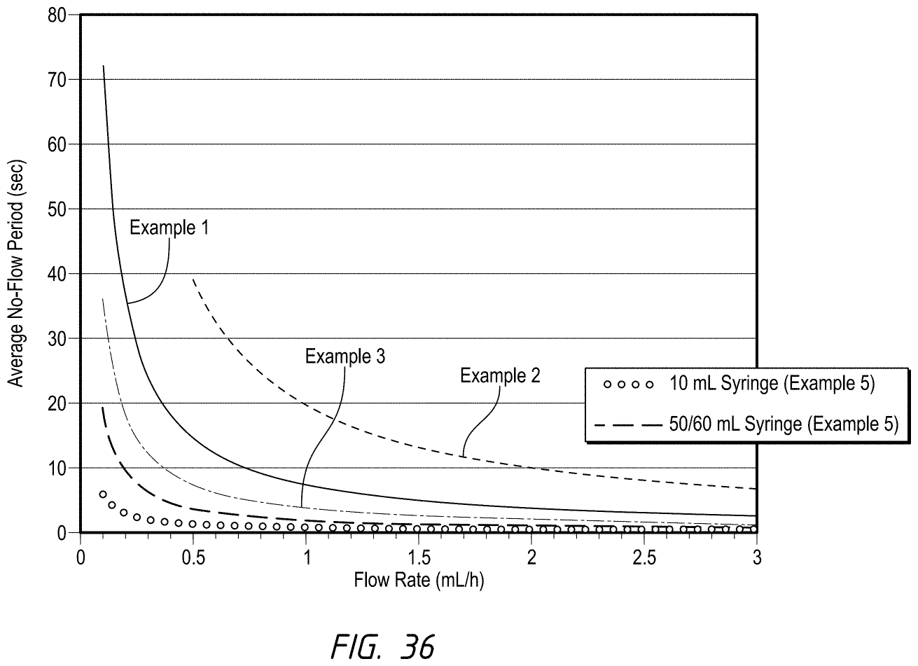

FIG. 36 shows a plot of theoretical syringe pump low flow continuity, as compared to three other non-syringe example commercial pumps. Example 5 is a syringe pump (which can operate with syringes of various sizes), so the two curves at the bottom left have lower average (and therefore better) no-flow periods than any of Examples 1, 2, or 3. The vertical axis is average no flow period in seconds, and the horizontal axis is flow rate in milliliters per hour. The no flow curves were calculated from resolution limit specifications.

| | | | | | | | Example 5 | Example 5 | |
|---|---|---|---|---|---|---|---|---|---|
| | Example 2 | | Example 3 | | Example 1 | | (10 cc) | (50 cc) | |
| Pump | | | 1 | μL | 2 | μL | 0.156 | 0.519 | μL |
| Resolution | | | | | | | | | |
| No-flow | N/A | | 35 | sec | 72 | sec | 6 sec | 19 | sec |
| period @ | | | | | | | | | |
| 0.1 mL/h | | | | | | | | | |
| No-flow | 39 | sec | 7 | sec | 14 | sec | | 4 | sec |
| period @ | | | | | | | | | |
| 0.5 mL/h | | | | | | | | | |

| | | | | | | | |
|---|---|---|---|---|---|---|---|
| No-flow | 19.5 | sec | 3.5 | sec | 7.2 | 2 | sec |
| period @ | | | | | | | |
| 1 mL/h | | | | | | | |

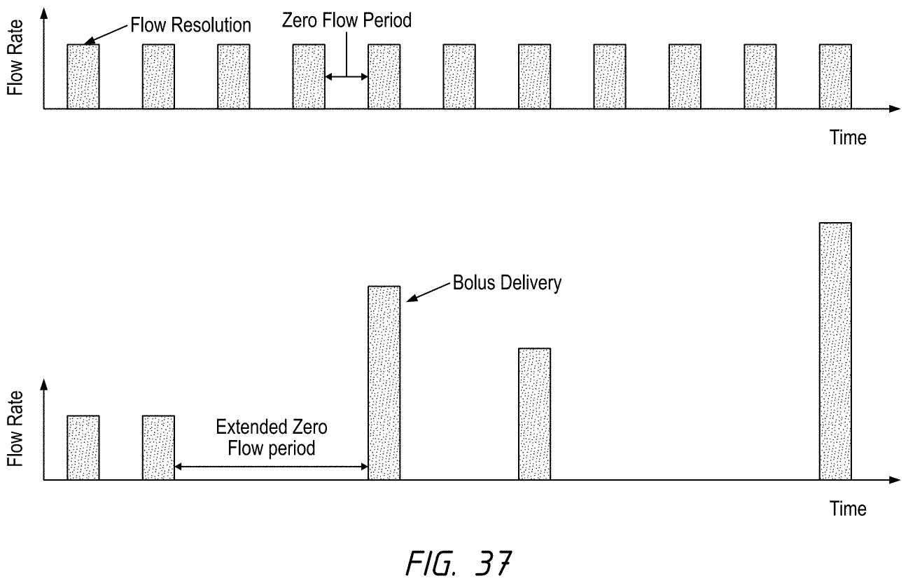

FIG. 37 shows how syringe pumps operate at low rates. Although syringe pumps may have theoretical no flow periods, stiction can be a problem for syringe pumps that undermines this theoretical advantage. Theoretically, each time the motor turns a small amount of fluid is infused; this is the flow resolution volume and is separated by “no flow” or “zero flow” periods, as shown at the top of FIG. 37. However, in reality, stiction within the syringe introduces inconsistencies including extended no-flow periods and boluses of fluid much larger than the target resolution. This is shown at the bottom of FIG. 37. This jerky movement due to stiction is difficult to predict or address systematically. Flow resolution for syringe pumps can generally improve (e.g., due to a lower minimum “fluid stroke”) in smaller syringes. However, continuity of delivery is less consistent than in large volume pumps due to syringe stiction. Rapid time to target flow rate and the more consistent flow profile generally helps make systems better for delivering some short half-life medications.

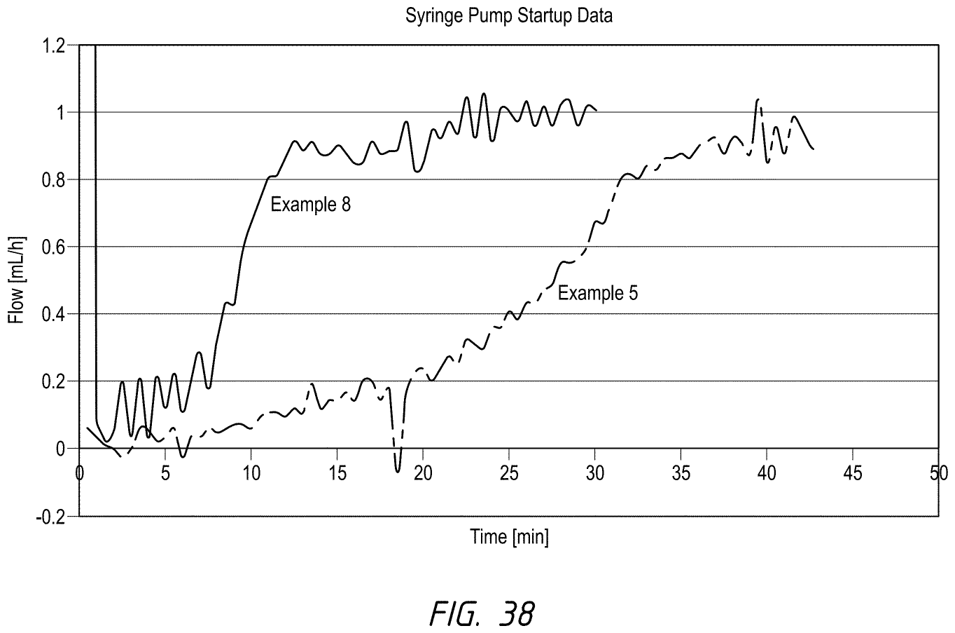

FIG. 38 shows startup curves at 1 mL per hour for two different commercial syringe pump examples. This data shows that syringe pumps can require a relatively long time (e.g., −13 min. for the upper curve and −32 min. for the lower curve) to reach target rates. These also show that actual infusion can follow irregular patterns, with wide and relatively rapid variations. This demonstrates that the time to deliver a medication to a patient can be excessive if the line is primed with another liquid that must be displaced before the medication of interest can be delivered to the patient. This directly shifts the time to initiate development of a medication level in a patient. Thus, the model of FIG. 29 (which assumes the bolus is delivered immediately and then starts to decay or decline due to biological processes) would need to be adjusted to assume a delay while the other liquid is displaced. During such a delay, the medication of interest has not yet reached the patient until the primed downstream volume has been displaced, and no patient response should yet be expected by the clinician.

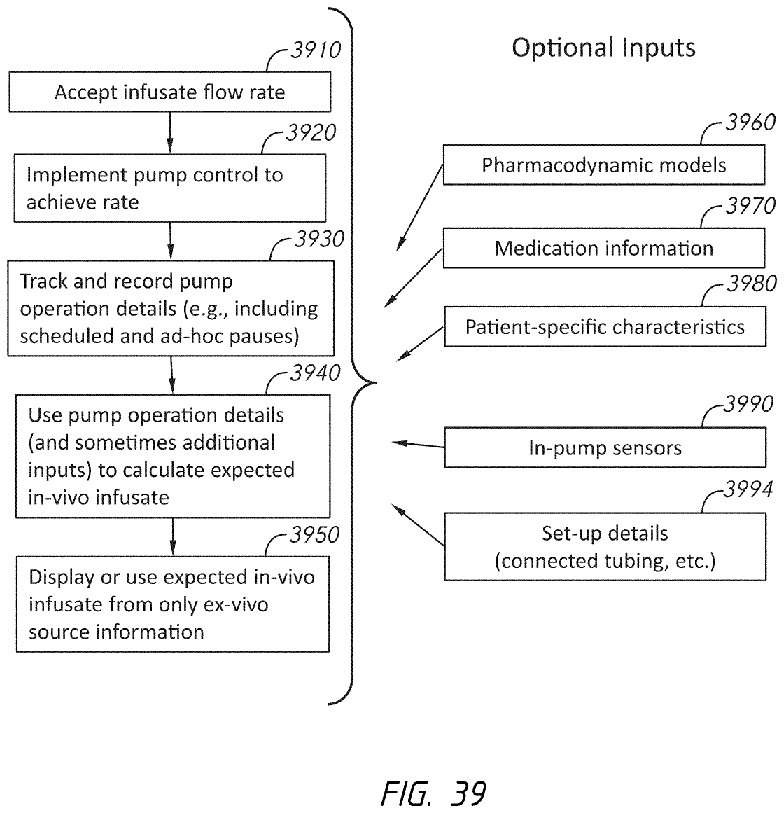

FIG. 39 shows a method where, at 3910, a device accepts an infusate flow rate. At 3920, the method can implement pump control in the device to achieve the selected flow rate. At 3930, the method can track and record pump operation details (e.g., in a device memory). This can include scheduled and ad hoc pauses. At 3940, the method can use those pump operation details, and sometimes additional inputs, to calculate (e.g., with a device processor) expected infusate amount currently present in-vivo. At 3950, the method can display (on a device interface) or use (e.g., through control feedback) expected in-vivo infusate amount, or could display the calculated % of amount as a function of eventual steady state, and/or time remaining to achieve imminent steady state under the current infusion. In some embodiments, some or all inputs for determining expected in-vivo infusate are from ex-vivo source information.

With further reference to FIG. 39, various optional inputs are shown. In addition to the pump operation details 3940, a system can have input from one or more pharmacodynamic models 3960. Such models can be supplemented by data from studies showing how medication is metabolized, absorbed, or its concentration decreases for other reasons in an infusate destination. This information can be stored and retrieved from a local or networked medication library database, for example. The system can also have input of medication information 3970. This is especially useful to distinguish between fast-metabolizing or rapidly absorbed medications and those that remain longer in the bloodstream or other infusate will remain destination. The system can also have input relating to patient-specific characteristics 3980. If a patient is receiving insulin, for example, that patient's insulin sensitivity may be relevant to how their body will use or react to that substance, and how much will remain present after an elapsed time. The system can also have input from in-pump sensors 3990. As described herein, control feedback can be greatly enhanced with such sensors, and they can provide feedback either directly from a pump motor or encoder, or from other sources such as a drip sensor that independently tracks the results from pump hardware movement. Alternatively, feedback can be provided without requiring “sensors”; it can come directly from a pump mechanism, for example, which can send information on how many rotations it has performed. The system can also have input from setup details 3994. Setup details may include a configuration, length, diameter, or other characteristics of connected tubing for example. A longer or wider tube may require more infusate (and a longer period) before infusate arrives at a destination (e.g., a patient's bloodstream).

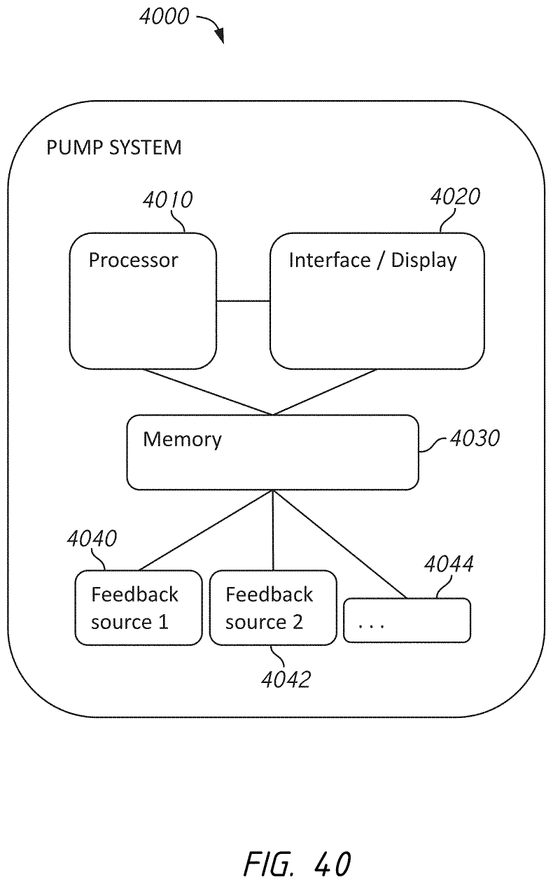

FIG. 40 shows a system 4000. The system 4000 can be a self-aware infusion pump and display system. Such a system can include a processor 4010 and a memory 4020. These can be configured to establish a known infusion volume history. This history can be established using stored device specific operation parameters to account for inherent pauses due to selection of low infusion rates. The history can be established using feedback from a user or sensors to account for inherent delays caused by how long it takes a drug or a change in the concentration of a drug to move from the pump through a catheter into a patient. The history can also be established by using system information regarding the duration of any pauses (e.g., those due to alarms, air bubble clearance, kink removal, or infusion bag or syringe replacement). The system can have a processor configured to use the known infusion volume history and an electronically stored drug database (e.g., that includes characteristic half-lives for particular drugs) to calculate present effective expected drug concentration. This can be conveyed to a clinician as a percentage of a desired (e.g., steady state) level—for example, showing a clinician that a patient's calculated drug level is 80% of the target equilibrium steady state. The system can also have a display configured to display to a clinician or other user the present effective calculated drug concentration, for example. The system may also distinguish: presentation of a delay in the medication reaching the patient (where no patient response should yet be expected); the medication reaching the patient but not yet expected to result in an equilibrium or steady state level in the patient; an expected steady state; and/or a temporary deviation from steady state that is being re-established.

The system 4000 can provides an example structure as schematically illustrated. A processor 4010 can interact with an interface/display 4020. Both of these components can interact with a memory 4030, which can include a drug database and can store a pump/flow history. The memory can receive input from feedback source 4040, feedback source 4042, and additional feedback sources 4044. These feedback sources can include onboard sensors within the pump system itself, and they can include inputs from an interface/display 4020, provided for example by a user. These inputs can also include information regarding a patient or a medication, for example from a hospital information system that is connected via network to the pump system 4000.

The system 4000 can be further configured to calculate and display an estimated time when a drug will first reach a patient, an estimated time when a drug load or concentration will reach a specified target level for example within a patient, and/or an estimated time when the patient is expected to achieve a particular physiological response to the drug. The system can be configured to be self-aware in the sense that it knows its own history, its own constraints, and how these are most likely to affect the results within an infusate destination—for example a patient's bloodstream.

The system 4000 can be configured to compensate for pauses in delivery of an infusate. This can be accomplished by infusing larger boluses of a drug into a patient within safe boundaries for concentration and timing, or it can be accomplished by infusing a drug at a constrained rate for a set amount of time or until a particular infusion goal is achieved. The system processor 4010 and memory 4020 can be further configured to facilitate prediction of future drug concentration by calculating extrapolated data points based on a trend line or other inputs, and the display 4020 can be configured to provide a graph to communicate the data points or trend line to a user. The system can be further configured to facilitate prediction of future drug concentration and automatically suggest or implement a flow rate change to avoid an undesired predicted future drug concentration. Memory 4030 can include a patient profile or other information relating to a specific treatment protocol or clinical history for a particular recipient for the infusate.

A system can comprise a noninvasive drug concentration estimator pump. The pump can have a memory configured to store a drug library, which can include multiple (e.g., two, three or more) fields selected from the following group: drug name, concentration or container volume, dosing unit, lower limit, upper limit, catch-up rate or dose permission, maximum catch-up rate or dose, drug half-life, drug expiration, and drug source. The memory can further be configured to store a patient profile having demographic, medical, or identifying data specific to the patient. The memory and/or one or more sensors or processors can be configured to track and record pump behavior. A processor can be configured to use the drug library, patient profile, and pump behavior to calculate predicted drug levels in the patient without input from in-vivo sensors. An interface can be configured to display the predicted drug levels and periodic pump behavior indicators. The pump behavior can be real-time input of forward fluid flow and paused fluid flow. Pump behavior can also be a measure or indicator of total volume infused.

An infusion pump can be configured to accept feedback on and account for numerous categories of information relating to its function and the expected results from any substances it infuses. For example, a pump can provide information (e.g., based on its own history) of expected in-patient amounts. It can track and account for infusion tube details, saline or other fluid carrier or “keep vessel open” flow effects, and any initial setup delays after infusion is initially requested. It can account for drug half-life (or more sophisticated medication models), elimination factors, and physiological responses. It can account for infusion pauses, including for bag replacement, air-in-line or occlusion alarms, etc. It can display related time-based information (past history, future projections, current levels, expected arrival time, expected response time).

### Described Examples

Various examples illustrate or embody the principles and technical advances of this disclosure. For example, a medical infusion pump system can comprise: an interface configured for selecting an infusate delivery rate; a pump configured to achieve the selected infusate delivery rates; a computer memory storing information that associates infusate delivery rates with pump operation details; a processor configured to accept the selected infusate delivery rate, access the computer memory, and use pump operation details to calculate expected infusate arrival time; and a user interface configured to provide a clinician with selected infusate delivery rate and an expected infusate arrival time.

In some embodiments, the pump can be further configured for intermittent mechanical movement having periodic pauses at low selected infusate delivery rates, and the computer memory is configured to store information that associates infusate delivery rates with pump operation details comprising length and frequency of periodic pump pauses.

In some embodiments, the system can further comprise feedback sensors positioned within the infusion pump, wherein the processor is further configured to accept input from these sensors and account for this input in the expected infusate arrival time provided through the user interface.

In some embodiments, the system can have computer memory storing information incorporating pharmacodynamic models specific to the type of medication being delivered, and the processor is further configured to account for this input and display in-patient medication level information through the user interface.

In some embodiments, the interface is further configured to accept set-up details comprising properties of connected tubing, the computer memory stores these set-up details, and the processor is further configured to account for this input in the expected infusate arrival time provided through the user interface.

In some embodiments, set-up details are received from a clinician through the user interface. In some embodiments, set-up details are received electronically through at least one of a wireless transmission or optical scan.

In some embodiments, the computer memory is further configured to store patient characteristics, and the processor is further configured to combine these characteristics with the pharmacodynamic models in calculating and displaying in-patient medication level information through the user interface.

In some embodiments, the patient characteristics are specific to a population of patients. In some embodiments, the patient characteristics are specific to an individual patient. In some embodiments, the patient characteristics are received from a hospital information system or the user interface and these details comprise a patient's sensitivity to a particular medication.

A self-aware infusion pump and display system can comprise: a processor and memory configured to establish a known infusion volume history by: using stored device-specific operation parameters to determine actual expected infusion rates; using feedback from a user or sensors to account for inherent delays caused by how long it takes the drug or a change in the concentration of a drug to move from the pump through a catheter into a patient; and using system information regarding the duration of any pauses due to alarms, air bubble clearance, kink removal, or infusion bag or syringe replacement. The processor can be configured to use the known infusion volume history and an electronically stored drug database comprising characteristic half-lives for particular drugs to calculate present effective expected drug concentration; and a display can be configured to display the present effective expected drug level to the user.

In some embodiments, using stored device-specific operation parameters to determine actual expected infusion rates further comprises using them to account for inherent pauses due to low selected infusion rates.

In some embodiments, the display is configured to display the present effective expected drug level with respect to a predicted or desired equilibrium level.

In some embodiments, the display is further configured to calculate and display: an estimated time when the drug will first reach the patient; an estimated time when the drug load or concentration will reach a specified target level; and an estimated time when the patient is expected to achieve a particular physiological response to the drug.

In some embodiments, the system is further configured to calculate and display an estimated time after which the drug is expected to remain at equilibrium under a constant flow rate, such that the infusion rate is approximately equal to the half-life break-down of the drug in the patient's blood.

In some embodiments, the processor is further configured to calculate that a medication has not yet reached a target level and the display provides a visual alert for promptly conveying this information to a user.

In some embodiments, the display is configured to provide the visual alert using at least one of a new icon or a modification to an existing display element.

In some embodiments, the target level comprises an in-patient equilibrium level of the drug.

In some embodiments, the processor is further configured to calculate that a medication is expected to have reached a target level and the display provides a visual alert for promptly conveying this information to a user.

In some embodiments, the system is further configured to compensate for pauses in the delivery. The system can compensate for pauses by infusing larger boluses of the drug into the patient within safe boundaries for concentration and timing.

In some embodiments, the processor calculates for the compensation based on recent pump activity and pharmacodynamic profile of the medication being infused.

In some embodiments, the processor and memory are further configured to facilitate prediction of future drug concentration by calculating extrapolated data points based on a trend line or other inputs, and the display is configured to convey this information through at least one of a percentage, a graph, or a trend line.

In some embodiments, the processor and memory are further configured to facilitate prediction of future drug concentration and automatically suggest or implement a flow rate change to improve a predicted future drug concentration.

A noninvasive drug level estimator pump can comprise: a memory configured to store a drug library, the drug library comprising a drug half-life and two or more fields selected from the following group: drug name, concentration or container volume, dosing unit, lower limit, upper limit, catch-up rate or dose permission, maximum catch-up rate or dose, drug expiration, and drug source. The memory can be further configured to track and record pump behavior. The pump can further comprise: a processor configured to use the drug library and pump behavior to calculate predicted drug levels in the patient without input from in-vivo sensors; and an interface configured to display the predicted drug levels and periodic pump behavior indicators.

In some embodiments, the processor is further configured to compare a drug expiration to an expected drug arrival time and the pump is configured to alert a user if the drug will expire before it is predicted to reach the patient.

In some embodiments, the memory is further configured to store a patient profile, the patient profile comprising demographic, medical, or identifying data specific to the patient.

In some embodiments, the pump behavior includes real-time information concerning forward fluid flow and paused fluid flow. In some embodiments, the pump behavior includes total volume infused.

A method of using pump hardware input to resume an equilibrium in-vivo medication level can comprise one or more of the following steps: using a pump interface to receive a medication infusion rate; using the medication infusion rate and a stored medication half-life to automatically calculate a target in-vivo medication range having an upper bound and a lower bound; advancing a pump based on the received rate and the target in-vivo medication range; using pump hardware to detect a non-standard infusion pause comprising a long interval that exceeds the length of a standard interval; in response to the detected pause, measuring the long interval and calculating a corresponding bolus amount of medication sufficient to achieve the upper bound for the target in-vivo medication range; and promptly after calculating, advancing the pump to infuse only the calculated bolus amount and then resuming standard pump advancement.

An infusion pump configured to determine and display drug load inside of a patient can comprise: a drug infusion rate module comprising an interface configured to accept a programmed rate and a memory configured to store the programmed rate; a drug decay module comprising a drug library with data on average drug levels over time for each drug; a pump pause module comprising hardware feedback sources; and an initial arrival module comprising an interface configured to accept user input on components connecting the pump to a drug destination.

In some embodiments, the pump can further comprise a processor configured to calculate and provide times when: the drug will reach the patient; the drug concentration will reach a specified level; or a physiological response to the drug is expected; calculate pump movement sufficient to compensate for pauses in the delivery by infusing larger amounts of the drug into the patient within safe boundaries for concentration and timing; and predict what the drug load or concentration will be in the patient over time after the infusion stops by providing a graph on the user interface.

In some embodiments, the pump further comprises a pump motor, wherein the processor and pump motor are further configured to act on the prediction by changing a flow rate or other parameters.

An intelligent medical infusion pump can comprise: a pumping chamber configured to contain medical fluid; a pump motor configured to actuate a rigid pumping element to advance medical fluid in the pumping chamber toward a patient; an interface configured to accept user input for selecting a medical fluid flow rate; a processor; a pump control unit; a memory configured to store: a user selected flow rate, a translation algorithm, and a pump operation history; the pump control unit, processor, and memory configured to use the translation algorithm to translate selected medical flow rates into electrical signals and control movement of the pump motor and the rigid pumping element to achieve the selected flow rate in the pumping chamber; and the processor and memory can be configured to use the pump operation history and pump configuration to provide output through the interface projecting at least one medical fluid timing event.

In some embodiments, the pump can be further configured such that: selection of a flow rate using the interface causes the pump control unit to send electrical signals pausing movement of the pump motor and rigid pumping element for at least ten seconds; the ten-second pause is recorded in the memory; the processor calculates the effect this pause will have on the at least one medical fluid timing event; and the interface displays this effect to a user.

In some embodiments, the fluid timing event comprises at least one of the following: time when the medical fluid has achieved equilibrium within a recipient; time when the recipient will exhibit a medical response to the medical fluid; remaining time until a maximum blood level for the medical fluid is reached; remaining time until a minimum blood level for the medical fluid is reached; remaining time until a safe blood level for the medical fluid is reached; remaining time until the medical fluid has cleared a recipient's system; remaining time until the recipient will stop exhibiting a medical response to the medical fluid; and remaining time until the recipient will no longer have the medical fluid in the recipient's blood stream.

In some embodiments, the pumping chamber comprises a resilient membrane, an outlet valve, and an inlet valve, the pumping element comprises a plunger, and the plunger is configured to periodically push against the resilient membrane to increase and decrease the pressure within the pumping chamber, causing alternating fluid flow through the inlet and outlet valves.

In some embodiments, the pump control unit comprises at least one of a gearbox and drive structure and at least one of an encoder and one or more sub-processors.

In some embodiments, the translation algorithm comprises using a table of empirical results of previous control signals and resulting measured rates.

In some embodiments, the memory is further configured to store a system configuration, and the pump control unit, processor, and memory are further configured to use the system configuration to provide output through the interface projecting at least one medical fluid timing event.

In some embodiments, the system configuration comprises a tube length between the pump and a medication recipient.

In some embodiments, the memory is further configured to store medication details, and the processor and memory are further configured to use the medication details to provide output through the interface projecting at least one medical fluid timing event.

In some embodiments, the medication details comprise an in-vivo medication rate accounting for at least one of metabolization, diffusion, and absorption.

In some embodiments, the medication details comprise an in-vivo medication rate accounting for at least one empirical data source, a published in-vivo half-life, or output from a two-compartment pharmacodynamic model for the relevant medical fluid.

In some embodiments, the medication details comprise one or more stored, sensed, or calculated physical properties selected from the group comprising: density, specific weight, specific volume, specific gravity, viscosity, and temperature.

In some embodiments, the memory is further configured to store patient-specific information selected from the group comprising sensitivity to medication, body temperature, heart rate, respiration rate, previous response history, medical conditions, cardiac output, and blood chemistry, and the processor and memory are further configured to use the patient-specific information to provide output through the interface projecting at least one medical fluid timing event.

In some embodiments, the pump has an internal pump feedback system configured to improve accuracy of flow rate and pump operation history, the feedback system including at least one of a flow sensor, a pressure sensor, an optical sensor, a piezoelectric sensor, an encoder, or a motor control loop element.

A method of using an infusion pump can include avoidance of in-vivo sensors by using only ex-vivo source information to display expected in-vivo infusate information.

A method of using a medical infusion pump can comprise: using a pump interface to accept a selected infusion rate; implementing periodic scheduled pump mechanism pauses to achieve the selected infusion rate; tracking and recording pump operation details in an operation history that includes the scheduled pump mechanism pauses and any ad-hoc pauses; using at least the operation history to calculate a destination expected infusate level; and using the destination expected infusate level to automatically control a function of the medical infusion pump.

In some embodiments, the function can comprise conveying the destination expected infusate level to a user through the pump interface.

In some embodiments, the method further comprises accepting information from at least one in-pump sensor and pump set-up details and using that information and those details to calculate a destination expected infusate level at a given time.

In some embodiments, the method further comprises accepting information from a database regarding infusate properties, destination-specific characteristics, and a pharmacodynamics model, and using that information to calculate the destination expected infusate level.

### Terminology and Conclusion

Reference throughout this specification to “some embodiments” or “an embodiment” means that a particular feature, structure or characteristic described in connection with the embodiment is included in at least some embodiments. Thus, appearances of the phrases “in some embodiments” or “in an embodiment” in various places throughout this specification are not necessarily all referring to the same embodiment and may refer to one or more of the same or different embodiments. Furthermore, the particular features, structures or characteristics may be combined in any suitable manner, as would be apparent to one of ordinary skill in the art from this disclosure, in one or more embodiments.

As used in this application, the terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Similarly, it should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Rather, inventive aspects lie in a combination of fewer than all features of any single foregoing disclosed embodiment.

Embodiments of the disclosed systems and methods may be used and/or implemented with local and/or remote devices, components, and/or modules. The term “remote” may include devices, components, and/or modules not stored locally, for example, not accessible via a local bus. Thus, a remote device may include a device which is physically located in the same room and connected via a device such as a switch or a local area network. In other situations, a remote device may also be located in a separate geographic area, such as, for example, in a different location, building, city, country, and so forth.

Methods and processes described herein may be embodied in, and partially or fully automated via, software code modules executed by one or more general and/or special purpose computers. The word “module” refers to logic embodied in hardware and/or firmware, or to a collection of software instructions, possibly having entry and exit points, written in a programming language, such as, for example, C or C++. A software module may be compiled and linked into an executable program, installed in a dynamically linked library, or may be written in an interpreted programming language such as, for example, BASIC, Perl, or Python. It will be appreciated that software modules may be callable from other modules or from themselves, and/or may be invoked in response to detected events or interrupts. Software instructions may be embedded in firmware, such as an erasable programmable read-only memory (EPROM). It will be further appreciated that hardware modules may be comprised of connected logic units, such as gates and flip-flops, and/or may be comprised of programmable units, such as programmable gate arrays, application specific integrated circuits, and/or processors. The modules described herein are preferably implemented as software modules, but may be represented in hardware and/or firmware. Moreover, although in some embodiments a module may be separately compiled, in other embodiments a module may represent a subset of instructions of a separately compiled program, and may not have an interface available to other logical program units.

In certain embodiments, code modules may be implemented and/or stored in any type of computer-readable medium or other computer storage device. In some systems, data (and/or metadata) input to the system, data generated by the system, and/or data used by the system can be stored in any type of computer data repository, such as a relational database and/or flat file system. Any of the systems, methods, and processes described herein may include an interface configured to permit interaction with patients, health care practitioners, administrators, other systems, components, programs, and so forth.

A number of applications, publications, and external documents may be incorporated by reference herein. Any conflict or contradiction between a statement in the body text of this specification and a statement in any of the incorporated documents is to be resolved in favor of the statement in the body text.

Terms of equality and inequality (less than, greater than) are used herein as commonly used in the art, e.g., accounting for uncertainties present in measurement and control systems. Thus, such terms can be read as approximately equal, approximate less than, and/or approximately greater than. In other aspects of the invention, an acceptable threshold of deviation or hysteresis can be established by the pump manufacturer, the editor of the drug library, or the user of a pump.

While the embodiments of the invention disclosed herein are presently considered to be preferred, various changes and modifications can be made without departing from the scope of the invention. Although described in the illustrative context of certain preferred embodiments and examples, it will be understood by those skilled in the art that the disclosure extends beyond the specifically described embodiments to other alternative embodiments and/or uses and obvious modifications and equivalents. Thus, it is intended that the scope of the claims which follow should not be limited by the particular embodiments described above. The scope of the invention is indicated in the appended claims, and all changes that come within the meaning and range of equivalents are intended to be embraced therein.

1-25. (canceled)

26. A noninvasive drug level estimator pump, the pump comprising:
a memory configured to store a drug library, the drug library comprising a drug half-life and two or more fields selected from the following group: drug name, concentration or container volume, dosing unit, lower limit, upper limit, catch-up dose permission, maximum catch-up dose, drug expiration, and drug source;
the memory further configured to track and record pump behavior;
a processor configured to use the drug library and pump behavior to calculate predicted drug levels in the patient without input from in-vivo sensors; and
an interface configured to display the predicted drug levels and periodic pump behavior indicators.

27. The pump of claim 26, wherein the processor is further configured to compare a drug expiration to an expected drug arrival time and the pump is configured to alert a user if the drug will expire before it is predicted to reach the patient.

28. The pump of claim 26, wherein the memory is further configured to store a patient profile, the patient profile comprising demographic, medical, or identifying data specific to the patient.

29. The pump of claim 26, wherein the pump behavior includes real-time information concerning forward fluid flow and paused fluid flow.

30. The pump of claim 29, wherein the pump behavior includes total volume infused.

31. A method of using pump hardware input to resume an equilibrium in-vivo medication level, the method comprising:
using a pump interface to receive a medication infusion rate;
using the medication infusion rate and a stored medication half-life to automatically calculate a target in-vivo medication range having an upper bound and a lower bound;
advancing a pump based on the received rate and the target in-vivo medication range;
using pump hardware to detect a non-standard infusion pause comprising a long interval that exceeds the length of a standard interval;
in response to the detected pause, measuring the long interval and calculating a corresponding amount of medication sufficient to achieve the upper bound for the target in-vivo medication range;
promptly after calculating, advancing the pump to infuse the calculated amount and then resuming standard pump advancement.

32. An infusion pump configured to determine and display drug load inside of a patient, the pump comprising:
a drug infusion rate module comprising an interface configured to accept a programmed rate and a memory configured to store the programmed rate;
a drug decay module comprising a drug library with data on average drug levels over time for each drug;
a pump pause module comprising hardware feedback sources; and
an initial arrival module comprising an interface configured to accept user input on components connecting the pump to a drug destination.

33. The pump of claim 32, further comprising a processor configured to:
calculate and provide times when:
the drug will reach the patient;
the drug concentration will reach a specified level; or
a physiological response to the drug is expected;
calculate pump movement sufficient to compensate for pauses in the delivery by infusing larger amounts of the drug into the patient within safe boundaries for concentration and timing; and
predict what the drug load or concentration will be in the patient over time after the infusion stops by providing a graph on the user interface.

34. The pump of claim 33, further comprising a pump motor, wherein the processor and pump motor are further configured to act on the prediction by changing a flow rate or other parameters.

35-53. (canceled)

54. The method of claim 31, wherein calculating a corresponding amount of medication comprises calculating a bolus amount, and advancing the pump to infuse the calculated amount comprises infusing only the calculated bolus before resuming standard pump advancement.

55. The method of claim 33, wherein the processor is configured to:
use the pump pause module and the initial arrival module to calculate and provide times when the drug will reach the patient; and
use the drug infusion rate module and the drug decay module to calculate and provide times when the drug will reach a specified level within the patient.

56. The method of claim 55, wherein the processor is further configured to calculate and provide times when a physiological response to the drug is expected.

57. The pump of claim 26, further comprising:
an interface configurable for selecting an infusate delivery rate;
a pump mechanism configured to achieve the selected infusate delivery rates;
the memory further configured to store information that associates infusate delivery rates with pump mechanism operation details;
the processor configured to accept the selected infusate delivery rate, access the memory, and use pump operation details to calculate expected infusate arrival time; and
a user interface configurable to provide a clinician with selected infusate delivery rate and an expected infusate arrival time.

58. The pump of claim 57, wherein the pump is further configured for intermittent mechanical movement having periodic pauses at low selected infusate delivery rates, and the memory is configured to store pump mechanism operation details comprising length and frequency of periodic pump pauses that have actually occurred.

59. The pump of claim 57, further comprising feedback sensors positioned within the infusion pump, wherein the processor is further configured to accept input from these sensors and account for this input in the expected infusate arrival time provided through the user interface.

60. The pump of claim 57, wherein the memory stores information incorporating pharmacodynamic models specific to the type of medication being delivered, and the processor is further configured to account for this input in displaying the predicted drug level through the user interface.

61. The pump of claim 57, wherein at least one interface of the pump is configured to accept set-up details received from at least one of a manual interface and electronically through at least one of an electronic transmission or optical scan, the set-up details comprising properties of connected tubing, the memory stores these set-up details, and the processor is further configured to account for this input in the expected infusate arrival time provided through the user interface.

62. The pump of claim 60, wherein the computer memory is further configured to store patient characteristics received from a user interface or a hospital information system, those received characteristics comprise a patient's sensitivity to a particular medication, and the processor is further configured to combine these characteristics with the pharmacodynamic models in calculating and displaying predicted drug levels through the at least one user interface.

63. The infusion pump of claim 32, wherein the drug library includes data accounting for drug decay from at least one of metabolization, diffusion, and absorption and:
taken from at least one empirical data source;
using a pulished in-vivo half-life; or
output from a two-compartment pharmacodynamics model.

64. The pump of claim 57, wherein the user interface is further configurable to provide at least one of the following:
the present effective expected drug level with respect to a predicted or desired equilibrium level;
a visual alert conveying that a drug has not yet reached a target level; and
a visual alert conveying that a medication is expected to have reached the target level.

65. The method of claim 31, further comprising compensating for the detected pause by infusing the calculated amount within safe boundaries for concentration and timing that are calculated based on recent pump activity and a pharmacodynamic profile of medication being infused.
